(19) United States
(12) Patent Application Publication
(10) Pub. No.: US 2026/0250683 A1
(43) Pub. Date: Aug. 27, 2026

(54) CONJUGATE COMPOSITIONS COMPRISING PHOSPHORYL GUANIDINE LINKAGES AND USES THEREOF

(71) Applicant: Avidity Biosciences, Inc., San Diego, CA (US)

(72) Inventors: Venkata Ramana DOPPALAPUDI, San Diego, CA (US); Michael Caramian COCHRAN, Palo Alto, CA (US); Subbarao NALLAGATLA, San Diego, CA (US); Giuseppe DELLO IACONO, Oceanside, CA (US); Sami Abdulwahab ABDULKADIR, San Diego, CA (US); Maryam JORDAN, Escondido, CA (US); Marc Andrew HARTMANN, San Diego, CA (US)

(21) Appl. No.: 19/405,243

(22) Filed: Dec. 1, 2025

Related U.S. Application Data
(63) Continuation of application No. PCT/US2024/032120, filed on May 31, 2024.
(60) Provisional application No. 63/505,925, filed on Jun. 2, 2023.

(51) Int. Cl. C12N 15/113; A61K 31/713; A61K 47/68; C07K 16/28

(52) U.S. Cl. CPC C12N 15/1135; A61K 31/713; A61K 47/6807; A61K 47/6849; C07K 16/2881; C12N 2310/14; C12N 2310/3145; C12N 2310/315; C12N 2310/345; C12N 2310/3513; C12N 2320/30

(57) ABSTRACT

Disclosed herein are compositions and pharmaceutical formulations that comprise a binding moiety conjugated to at least one oligonucleotide. In some cases, the oligonucleotide comprises at least one modified internucleotide linkage. Also, described herein include methods for treating a disease which utilize a composition or a pharmaceutical formulation comprising a binding moiety conjugated to at least one oligonucleotide.

### CROSS-REFERENCE

This application is a continuation of International Application PCT/US2024/032120, filed on May 31, 2024, which claims the benefit of U.S. Provisional Application No. 63/505,925, filed Jun. 2, 2023, each of which is incorporated by reference herein in its entirety.

### SEQUENCE LISTING

The instant application contains a Sequence Listing which is being submitted electronically in XML format and is hereby incorporated by reference in its entirety. Said XML copy, created on May 6, 2026, is named 72904-707_301_SL.xml and is 226,775 bytes in size.

### BACKGROUND OF THE DISCLOSURE

Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long-lasting effect over multiple cell divisions. As such, RNAi provides a viable method for drug target validation, gene function analysis, pathway analysis, and disease therapeutics.

Phosphodiester (PO) or phosphorothioate (P=S) internucleotide linkages are used for linking nucleotides in a single- and double-stranded nucleotides, e.g., inhibitory nucleic acids, double-stranded siRNAs, or single-stranded antisense oligonucleotides (ASO). P=S internucleotide linkages are used to replace PO linkages on single- and double-stranded nucleotides to improve their stability by enhancing resistance against nuclease. However, the presence of two or more single- and double-stranded nucleotides (e.g., siRNAs) with P=S linkages conjugated to an antibody (e.g., drug-to-antibody ratio of 2:1 (DAR2) conjugate or DAR2 antibody-oligonucleotide conjugate (AOC)) decreases the activities and stabilities of the DAR2 conjugates in vivo. The loss of in vivo biological activity is due to the rapid clearance of the DAR2 conjugates likely caused by the negative charges of the P=S linkages of the two siRNA molecules conjugated to an antibody. Thus, there is a need to improve the chemistry of the internucleotide linkages by decreasing the negative charges of the two siRNAs to improve activities and stabilities of the DAR2 AOC conjugates.

### SUMMARY OF THE DISCLOSURE

In the present disclosure, methods and compositions to reduce the negative charges of the inhibitory nucleic acid (e.g., siRNA, ASO, phosphorodiamidate morpholino oligomers (PMO), etc.) are provided by decreasing the number of phosphorothioate (P=S) internucleotide linkages in the inhibitory nucleic acid (e.g., siRNA, ASO, PMO, etc.) molecules and/or substituting the phosphorothioate (P=S) internucleotide linkages with phosphoryl guanidine (PG) internucleotide linkages since PG is a neutral charge while P=S is a negative charge. In addition, the present disclosure provides methods and compositions to modify inhibitory nucleic acid (e.g., siRNA, ASO, PMO, etc.) by substituting PO linkages with PG linkages to increase the number of PG linkages in the inhibitory nucleic acid (e.g., siRNA, ASO, PMO, etc.) molecule. The presence of PG internucleotide linkages on the two inhibitory nucleic acid (e.g., siRNA, ASO, PMO, etc.) molecules in the DAR2 AOC improves activities and stabilities of DAR2 AOC.

In one aspect, provided herein are conjugates of Formula (I):

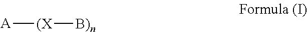

wherein,
A is a binding moiety;
B is a double-stranded oligonucleotide comprising a guide strand and a passenger strand;
X is a bond or a linker;
n is a number≥2; and
wherein the guide strand or the passenger strand comprises at least one modified internucleotide linkage comprising the structure of Formula (II):

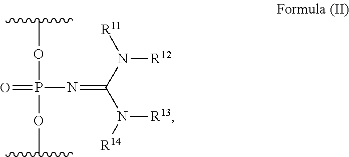

wherein each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl;
optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine.
   In some aspects, R¹² and R¹³, together with the atom to which they are bound, form an imidazolidine. In some aspects, the internucleotide linkage has the structure of Formula (III):

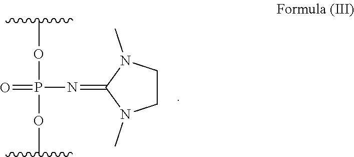 [SMILES: *OP(=O)(N=C1N(C)CCN1C)O*]

In some aspects, the internucleotide linkage of Formula (III) is a phosphoryl guanidine (PG) linkage. In some aspects, R¹¹, R¹², R¹³, and R¹⁴ are —C₁₋₁₀ alkyl. In some aspects, the internucleotide linkage has the structure of Formula (IV):

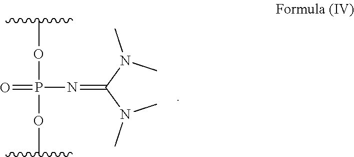 [SMILES: *OP(=O)(N=C(N(C)C)N(C)C)O*]

In some aspects, the at least one modified internucleotide linkage of Formula (II) is located at the 5′ or 3′ end of the guide strand. In some aspects, the at least one modified internucleotide linkage of Formula (II) is located at an internal position of the guide strand. In some aspects, the at least one modified internucleotide linkage of Formula (II) is located at the 3′ overhang of the double-stranded oligonucleotide. In some aspects, the guide strand further comprises at least one phosphorothioate internucleotide linkage. In some aspects, the at least one phosphorothioate internucleotide linkage and the at least one modified internucleotide linkage comprising the structure of Formula (II) are adjacent to one another. In some aspects, the passenger strand comprises the at least one modified internucleotide linkage of Formula (II). In some aspects, the passenger strand has up to 18 internucleotide linkages of Formula (II). In some aspects, the at least one modified internucleotide linkage of Formula (II) is not located at the cut site of the passenger strand. In some aspects, the at least one modified internucleotide linkage of Formula (II) is located at the 5′ or 3′ end of the passenger strand. In some aspects, the at least one modified internucleotide linkage of Formula (II) is located at an internal position of the passenger strand. In some aspects, the double-stranded oligonucleotide further comprises at least one 2′ modified nucleotide or at least one inverted abasic moiety. In some aspects, the passenger strand comprises two modified internucleotide linkages of Formula (II) at the 3′ end and two modified internucleotide linkages of Formula (II) at the 5′ end. In some aspects, the passenger strand comprises four modified internucleotide linkages of Formula (II) at the 3′ end and four modified internucleotide linkages of Formula (II) at the 5′ end. In some aspects, the passenger strand comprises 18 modified internucleotide linkages of Formula (II). In some aspects, the passenger strand comprises two modified internucleotide linkages of Formula (II) at the 3′ end and two modified internucleotide linkages of Formula (II) at the 5′ end, and wherein the guide strand comprises two modified internucleotide linkages of Formula (II) at the 3′ end and two modified internucleotide linkages of Formula (II) at the 5′ end. In some aspects, the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified nucleotide. In some aspects, the at least one 2′ modified nucleotide comprises locked nucleic acid (LNA) or ethylene nucleic acid (ENA). In some aspects, the at least one inverted abasic moiety is at least one terminus. In some aspects, the at least one 2′ modified nucleotide comprises at least one non-natural nucleotide. In some aspects, the double-stranded oligonucleotide further comprises a 5′ terminal vinylphosphonate modified nucleotide. In some aspects, the double-stranded oligonucleotide further comprises a modified internucleotide linkage selected from an alkylphosphonate, a triester, or a mesyl phosphoramidiate. In some aspects, X is a bond. In some aspects, X is a C₁-C₆ alkyl group. In some aspects, X is a homobifunctional linker or a heterobifunctional linker, optionally conjugated to a C₁-C₆ alkyl group. In some aspects, X is a heterobifunctional linker. In some aspects, the heterobifunctional linker is succinimidyl-4-(N-maleimidomethyl)cyclohexane-1-carboxylate (sMCC), optionally conjugated to a C₁-C₆ alkyl group. In some aspects, X is a cleavable linker. In some aspects, X is a non-cleavable linker. In some aspects, the binding moiety is selected from the group consisting of a polypeptide, a protein, or an antibody or antigen binding fragment thereof. In some aspects, the binding moiety is an antibody or antigen binding fragment thereof. In some aspects, the antibody or antigen binding fragment thereof binds to a cell surface receptor. In some aspects, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In some aspects, the conjugate has a drug to antibody ratio (DAR) of about 2:1, 3:1 or 4:1. In some aspects, the modified internucleotide linkage of Formula (II) on the guide strand or passenger strand increases activity of the conjugate of Formula (I). In some aspects, the modified internucleotide linkage of Formula (II) on the guide strand or passenger strand of the siRNA increases the stability of the conjugate of Formula (I).

In another aspect, provided herein are methods of modulating mRNA expression levels of a gene in a subject, the method comprising: providing the conjugate disclosed herein; and administering to the subject the conjugate, wherein the conjugate decreases mRNA expression levels of the gene in the subject. In some aspects, the conjugate decreases the expression levels of the gene by at least about 20%, 25%, 30%, 35%, 40%, 45%, 50%, 55%, 60%, 65%, 70%, 75%, 80%, 85%, 90%, or 95% relative to a control sample. In some aspects, the conjugate has an increased plasma half-life relative to an analogous conjugate comprising phosphorothioate internucleotide linkages exchanged at the positions of the modified internucleotide linkages of Formula (II).

In another aspect, provided herein are methods of treating muscle atrophy or myotonic dystrophy in a subject in need thereof, the method comprising: providing the conjugate disclosed herein; and administering to the subject the conjugate, wherein the conjugate mediates RNA interference against a target mRNA in the subject, thereby treating muscle atrophy or mytonic dystrophy in the subject.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a SAX chromatogram for DAR2 conjugates (DAR2 AOC) having myostatin (MSTN) siRNA modified with 8 phosphoryl guanidine (PG) linkages (R3668).

FIG. 2 shows a SAX chromatogram for DAR2 conjugates (DAR2 AOC) having MSTN siRNA modified with 12 PG linkages (R3669).

FIG. 3 shows mass spectrometry data of molecular weight (MW) for MSTN passenger strand (PS) with 8 PG linkages.

FIG. 4 shows mass spectrometry data of MW for MSTN PS with 12 PG linkages.

FIG. 5 shows mass spectrometry data of MW for MSTN guide strand (GS) with vinyl phosphonate vpUq.

FIG. 6 shows ion-pair reverse phase (IP-RP) chromatogram of MSTN GS with vinyl phosphonate vpUq.

FIG. 7 shows an IP-RP chromatogram of MSTN PS with 8 PG linkages.

FIG. 8 shows an IP-RP chromatogram of MSTN PS with 12 PG linkages.

FIG. 9 shows a non-denaturing IP-RP of MSTN duplex with 8 PG linkages.

FIG. 10 shows a non-denaturing IP-RP of MSTN duplex with 12 PG linkages.

FIG. 11 shows SAX-HPLC of MSTN duplex with 8 PG linkages.

FIG. 12 shows SAX-HPLC of MSTN duplex with 12 PG linkages.

FIGS. 13A and 13B show the in vivo dose response of MSTN mRNA expression levels in the gastrocnemius muscle of mice administered with increasing concentrations of DAR2 AOC with PG modified siRNA (2, 4, 8, and 15 mg/kg of mAb) at Day 28 (FIG. 13A) and the in vivo time-dependent response of MSTN mRNA expression levels in the gastrocnemius muscle in mice administered with a dose of DAR2 AOC with PG modified siRNA (7.5 mg/kg of mAb) at Day 14, 28, and 56 (FIG. 13B).

FIGS. 14A and 14B show six different sets of in vivo MSTN mRNA expression levels in the gastrocnemius muscle of mice administered with a single dose of DAR2 AOC with PG modified siRNA (7.5 mg/kg of mAb) at Day 28 (FIG. 14A) and the distribution summary of the MSTN mRNA expression levels in the gastrocnemius muscle of mice administered with a single dose of DAR2 AOC with PG modified siRNA (7.5 mg/kg of mAb) at Day 28 (FIG. 14B).

FIGS. 15A and 15B show a time-dependent plasma concentration of siRNA as the percentage of the single dose of DAR2 AOC with PG modified siRNA (10 mg/kg of mAb) administered to mice at 24, 72, and 168 hours post injection (FIG. 15A) and the calculated area under the curve (AUC) for the PG modified siRNA after 168 hours post injection (FIG. 15B).

FIGS. 16A-16D show the in vivo SSB mRNA expression levels in the gastrocnemius or heart muscle of mice administered with of DAR2 AOC (5 mg/kg of mAb) with siRNA modified with 0, 4, 8, or 12 PG on the passenger strand at Day 28 (FIGS. 16A and 16B) and the plasma concentration of SSB siRNA as the percentage of the single dose of DAR2 AOC (5 mg/kg of mAb) with siRNA modified with 0 or 8 PG administered to mice at 6 hours post injection (FIG. 16C) and the plasma concentration of MSTN siRNA as the percentage of the single dose of DAR2 AOC (5 mg/kg of mAb) with siRNA modified with 0 PG, 4PG, 8 PG, or 15 PG administered to mice at 6 hours post injection (FIG. 16D).

FIG. 17A shows the in vivo MSTN mRNA levels in the gastrocnemius muscle of mice administered with a single dose of DAR2 AOC with PG modified siRNA (7.5 mg/kg of mAb) at Day 28. FIG. 17B shows the Day 14 tissue concentration (in nM) of siRNA after dosing mice with DAR2 AOC with PG modified siRNA (5 mg/kg of mAb).

FIG. 18A shows the in vivo MSTN mRNA levels in the gastrocnemius muscle of mice administered with a single dose of DAR2 AOC with PG modified siRNA (7.5 mg/kg of mAb) at Day 14 (left) and Day 28 (right). FIG. 18B shows the Day 14 (left) and Day 28 (right) tissue concentrations (in mM) of siRNA in gastrocnemius muscle after dosing mice with DAR2 AOC with PG modified siRNA (5 mg/kg of mAb).

FIG. 19 shows the in vivo MSTN mRNA levels in the gastrocnemius muscle of mice administered with a single dose of DAR2 AOC with siRNA modified with 8 PG on the passenger strand (7.5 mg/kg of mAb) at Day 28.

FIG. 20 shows the in vivo MSTN mRNA levels in the gastrocnemius muscle of mice administered with a single dose of DAR2 AOC with siRNA modified with 1 or 12-15 PG on the passenger strand (7.5 mg/kg of mAb) at Day 28.

FIGS. 21A and 21B show the in vivo MSTN mRNA levels in the gastrocnemius muscle of mice administered with a single dose of DAR2 AOC with 0, 4, 8, or 12 PG modified siRNA (7.5 mg/kg of mAb) at Day 14 or Day 28 (FIG. 21A) and the in vivo MSTN mRNA levels expression in the gastrocnemius muscle of mice administered with a single dose of DAR2 AOC with 0, 4, 6, or 8 modified siRNA (7.5 mg/kg of mAb) at Day 28 (FIG. 21B).

FIG. 22 illustrates the locations of the internucleotide linkages that were modified with phosphoryl guanidine (PG) linkages on the guide strand, passenger strand, or both strands of the two siRNAs of the AOCs.

FIG. 23 illustrates the locations of the internucleotide linkages that were modified with phosphoryl guanidine (PG) groups on the guide strand, passenger strand, or both strands of the two siRNAs of the DAR2 AOCs.

### DETAILED DESCRIPTION OF THE DISCLOSURE

### Therapeutic Molecule Platform

Nucleic acid-based therapy (e.g., RNAi) is a targeted therapy with high selectivity and specificity. However, in some instances, nucleic acid-based therapy is hindered by poor intracellular uptake, insufficient intracellular concentrations in target cells, low efficacy, and low stability in vivo. To address these issues, various modifications of the nucleic acid composition are explored including modification of internucleotide linkages to increase the stability of the nucleic acid molecules. Disclosed herein, in some aspects, are structures of modified internucleotide linkages (e.g., phosphoguanidine (PG) internucleotide linkages) that can be incorporated into the oligonucleotide (or polynucleic acids). In some aspects, the modified internucleotide linkages described herein can substitute one or more of the phosphodiester linkages or one or more of the phosphorothioate (P=S) internucleotide linkages of the DNA, RNA, DNA/RNA hybrid, or synthetic oligonucleotides. In some aspects, the internucleotide linkages described herein increase the stability or half-life of the oligonucleotide in vivo. In some aspects, the internucleotide linkages described herein increase the stability or half-life of the oligonucleotides in vivo without any substantial or significant changes in the toxicity.

### Drug Conjugates

In some aspects, disclosed herein are drug conjugates (e.g., a therapeutic oligonucleotide conjugate). In some aspects, disclosed herein is a conjugate of Formula (I):

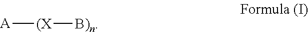

In some aspects, A is a binding moiety, B is a drug, and X is a bond or a linker. In some aspects, B is an oligonucleotide drug. In some aspects, the oligonucleotide drug is a single-stranded oligonucleotide (e.g., single-stranded antisense oligonucleotide, etc.). In some aspects, the oligonucleotide drug is a double-stranded oligonucleotide (e.g., dsRNA, siRNA, etc.). In some aspects, the drug is the oligonucleotide drug comprising at least one modified internucleotide linkage comprising the structure of Formula (II). In some aspects, the oligonucleotide drug is a double-stranded oligonucleotide (e.g., siRNA), and at least one or both strands (guide strand and/or passenger strand) of the double-stranded oligonucleotide comprise at least one modified internucleotide linkage comprising the structure of Formula (II). In some aspects, the number of the drug molecules coupled or conjugated to a single binding moiety is at least 2 or at least about 2. In some aspects, the average number of the drug molecules coupled or conjugated to a single binding moiety molecule in the composition is at least 2 or at least about 2. In some aspects, the average number of the drug molecules coupled or conjugated to a single binding moiety molecule in the composition is at least about 2, 3, 4, 5, 6, 7, 8, 9, 10, 11, 12, 13, 14, 15, 16, or greater. In some aspects, the average number of the drug molecules coupled or conjugated to a single binding moiety molecule in the composition is about 2-12, about 2-10, about 2-8, about 4-8, or about 6-8.

### Oligonucleotide Drug Molecule

In some aspects, the drug in the drug-binding moiety conjugate is an oligonucleotide drug (polynucleic acid drug). In some aspects, the oligonucleotide drug is an RNA oligonucleotide, a DNA oligonucleotide, or an RNA/DNA hybrid oligonucleotide. In some aspects, the oligonucleotide drug is a synthetic oligonucleotide. In some aspects, the oligonucleotide drug is a modified RNA oligonucleotide, a DNA oligonucleotide, or an RNA/DNA hybrid oligonucleotide. In some instances, RNA oligonucleotide comprises small interfering RNA (siRNA), short hairpin RNA (shRNA), microRNA (miRNA), and double-stranded RNA (dsRNA).

In some aspects, the oligonucleotide is a single-stranded oligonucleotide. In some aspects, the oligonucleotide is a single-stranded antisense oligonucleotide. In some aspects, the oligonucleotide is a single-stranded phosphorodiamidate morpholino oligomer (PMO). In some instances, the oligonucleotide described herein comprises a single-stranded nucleic acid of about 15-50, 15-40, 15-35, 15-30, 15-25, 15-20, 20-30, 25-30, 16-30, 17-30, 18-30, 18-27, 18-25, 18-23, 19-23, 20-23, or 21-23 nucleotides in length. In some aspects, the oligonucleotide comprises a single-stranded nucleic acid of about 15, 16, 17, 18, 19, or 20 nucleotides long. In some aspects, the oligonucleotide comprises a single-stranded nucleic acid of about 21, 22, 23, 24, or 25 nucleotides long. In some aspects, the oligonucleotide comprises a single-stranded nucleic acid of about 26, 27, 28, 29, or 30 nucleotides long. In some instances, the oligonucleotide described herein comprises a single-stranded nucleic acid of about 21 nucleotides long. In some instances, the oligonucleotide described herein comprises a single-stranded oligonucleotide of about 23 nucleotides long.

In some aspects, the oligonucleotide is a double-stranded oligonucleotide. In some aspects, the oligonucleotide is a dsRNA. In some aspects, the oligonucleotide is an siRNA. In some instances, the double-stranded oligonucleotide comprises a sense strand and an antisense strand. As used herein, the term “sense strand” can be interchangeably used with the term “passenger strand,” and the term “antisense strand” can be interchangeably used with the term “guide strand.” The sense and antisense strand are at least partially complementary with each other to form a double-stranded oligonucleotide. In some instances, each strand of the double-stranded oligonucleotide comprises about 19, 20, 21, 22, 23, 24, or 25 nucleotides long. In some instances, one strand of the double-stranded oligonucleotides is 1, 2, 3 nucleotides longer than the other strand, thereby forming an overhang structure at its 5′ or 3′ end.

In some aspects, described herein is oligonucleotide with modifications. In some aspects, the oligonucleotide comprises natural or synthetic or artificial nucleotide analogues or bases. In some cases, the oligonucleotide comprises combinations of DNA, RNA and/or synthetic or artificial nucleotide analogues (e.g., with ribose ring modification (e.g., open ring structure, locked ring structure, LNA, GNA, PNA, TNA, etc.), with base modification or abasic analogues, or combination thereof). In some instances, the synthetic or artificial nucleotide analogues or bases comprise modifications at one or more of ribose moiety, phosphate moiety, nucleoside moiety, or a combination thereof. In some instances, the oligonucleotide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more modifications. In some instances, the oligonucleotide comprises nucleic acid sequences, at least 10%, 20%, 30%, 40%, 50%, 60%, 70%, 80%, or 90% of which comprises one or more modifications. In some instances, the oligonucleotide comprises nucleic acid sequences that are fully modified.

In some aspects, the double-stranded oligonucleotide comprising a guide strand and a passenger strand, wherein the guide strand or the passenger strand comprises at least one modified internucleotide linkage comprising the structure of Formula (II), wherein each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl, and optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine. In some aspects, R¹² and R¹³, together with the atom to which they are bound, form an imidazolidine. In some aspects, the internucleotide linkage has the structure of Formula (III).

In some aspects, R¹¹, R¹², R¹³, and R¹⁴ are —C₁₋₁₀ alkyl. In some aspects, the internucleotide linkage has the structure of Formula (IV).

In some aspects, the at least one modified internucleotide linkage of Formula (II) is located at the 5′ or 3′ end of the guide strand. In some aspects, the at least one modified internucleotide linkage of Formula (II) is located at an internal position of the guide strand. In some aspects, the at least one modified internucleotide linkage of Formula (II) is located at the 3′ overhang of the double-stranded oligonucleotide. In some aspects, the at least one modified internucleotide linkage of Formula (II) is not located at the cut site of the passenger strand. In some aspects, the at least one modified internucleotide linkage of Formula (II) is located at the 5′ or 3′ end of the passenger strand. In some aspects, the at least one modified internucleotide linkage of Formula (II) is located at an internal position of the passenger strand.

### Modified Internucleotide Linkages

In some aspects, an oligonucleotide disclosed herein comprises at least one internucleotide linkage represented by Formula (II):

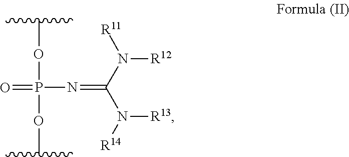

wherein,
each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl;
optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine.

In some aspects, R¹² and R¹³, together with an atom to which they are bound, form a 5-8 membered heterocyclic substituent moiety. In some aspects, R¹² and R¹³, together with an atom to which they are bound, form a 5-membered heterocyclic substituent moiety. In some aspects, R¹² and R¹³, together with an atom to which they are bound, form a 6-membered heterocyclic substituent moiety. In some aspects, R¹² and R¹³, together with an atom to which they are bound, form a 7-membered heterocyclic substituent moiety. In some aspects, R¹² and R¹³, together with an atom to which they are bound, form an 8-membered heterocyclic substituent moiety.

In some aspects, R¹² and R¹³, together with the atom to which they are bound to form N-pyrrolidinyl. In some aspects, R¹² and R¹³, together with the atom to which they are bound, form N-piperidinyl. In some aspects, R¹² and R¹³, together with the atom to which they are bound, form N-azepanyl. In some aspects, R¹² and R¹³, together with the atom to which they are bound, form N-azocanyl. In some aspects, R¹² and R¹³, together with the atom to which they are bound, form an imidazolidine.

In some aspects, each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl. In some aspects, R¹¹ is selected from —H, —C₁₋₁₀ alkyl, —C₂₋₁₀ alkenyl, —C₂₋₁₀ alkynyl, or —C₆₋₁₀ aryl. In some aspects, R¹¹ is methyl. In some aspects, R¹² is selected from —H, —C₁₋₁₀ alkyl, —C₂₋₁₀ alkenyl, —C₂₋₁₀ alkynyl, or —C₆₋₁₀ aryl. In some aspects, R¹² is methyl. In some aspects, R¹³ is selected from —H, —C₁₋₁₀ alkyl, —C₂₋₁₀ alkenyl, —C₂₋₁₀ alkynyl, or —C₆₋₁₀ aryl. In some aspects, R¹³ is methyl. In some aspects, R¹⁴ is selected from —H, —C₁₋₁₀ alkyl, —C₂₋₁₀ alkenyl, —C₂₋₁₀ alkynyl, or —C₆₋₁₀ aryl. In some aspects, R¹⁴ is methyl.

In some aspects, R¹¹, R¹², R¹³, and R¹⁴ are —C₁₋₁₀ alkyl. In some aspects, R¹¹ is —C₁₋₁₀ alkyl. In some aspects, R¹¹ is methyl. In some aspects, R¹² is —C₁₋₁₀ alkyl. In some aspects, R¹² is methyl. In some aspects, R¹³ is —C₁₋₁₀ alkyl. In some aspects, R¹³ is methyl. In some aspects, R¹⁴ is —C₁₋₁₀ alkyl. In some aspects, R¹⁴ is methyl.

In some aspects, an oligonucleotide comprises at least one internucleotide linkage represented by Formula (III):

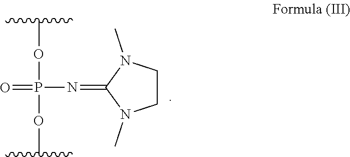 [SMILES: *OP(=O)(N=C1N(C)CCN1C)O*]

In some aspects, an oligonucleotide comprises at least one internucleotide linkage represented by Formula (IV):

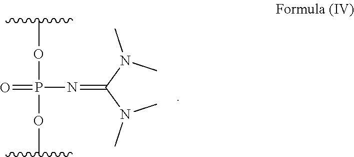 [SMILES: *OP(=O)(N=C(N(C)C)N(C)C)O*]

In some aspects, an oligonucleotide comprises at least one internucleotide linkage represented by Formula (V):

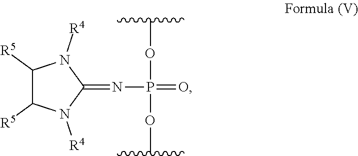

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, an oligonucleotide comprises at least one internucleotide linkage represented by Formula (V):

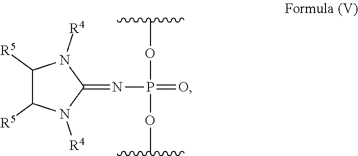

wherein each R⁴ is independently selected from hydrogen or C₁₋₁₀ alkyl; and
wherein the oligonucleotide comprises at least 12 nucleotides.

In some aspects, an oligonucleotide comprises a contiguous sequence of monomer subunits linked by internucleotide linking groups wherein at least one of the internucleotide linking groups is represented by Formula (V):

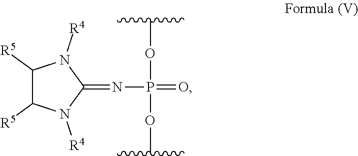

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, a second oligonucleotide comprises a contiguous sequence of monomer subunits linked by internucleotide linking groups wherein at least one of the internucleotide linking groups is represented by Formula (V):

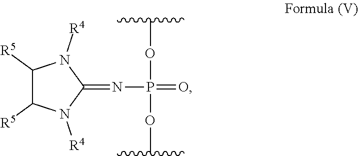

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, an oligonucleotide includes at least one monomer subunit of the monomer subunits is represented by Formula (V*):

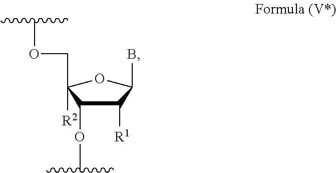

wherein,
B is independently selected from a heterocyclic base moiety;
R¹ is independently selected from hydrogen, hydroxy, halogen, or alkoxy;
R² is independently hydrogen; and
R¹ and R² optionally come together with the atoms to which they are bound to form a C₃-C₄ carbocycle.

In some aspects, an oligonucleotide includes at least two monomer subunits of the contiguous sequence are linked by Formula (V) as represented by Formula (VI):

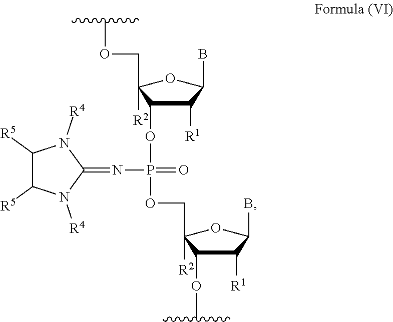

wherein,
each B is independently selected from a heterocyclic base moiety;
each R¹ is independently selected from hydrogen, hydroxy, halogen, or alkoxy;
each R² is independently hydrogen;
R¹ and R² optionally come together with the atoms to which they are bound to form a C₃-C₄ carbocycle;
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, each R¹ is independently selected from hydrogen, hydroxy, halogen, or alkoxy. In some cases, R¹ is hydrogen. In some cases, R¹ is hydroxy. In some cases, R¹ is halogen. In some cases, R¹ is fluorine. In some cases, R¹ is C₁₋₆ alkoxy.

In some aspects, each R⁴ is independently selected from hydrogen or C₁₋₆ alkyl. In some aspects, each R⁴ is independently selected from hydrogen. In some aspects, each R⁴ is independently selected from C₁₋₆ alkyl.

In some aspects, R¹ and R² optionally come together with the atoms to which they are bound to form a C₃ carbocycle. In some cases, R¹ and R² optionally come together with the atoms to which they are bound to form a C₄ carbocycle.

In some aspects, the heterocyclic base moiety is a modified base as described elsewhere herein.

In some aspects, each internucleotide linkage is represented by Formula (II), (III), (IV), or (V).

In some aspects, a modified internucleotide linkage is a phosphoryl guanidine (PG) linkage of Formula (II), (III), (IV), or (V). In some aspects, the modified internucleotide linkage is a phosphoryl guanidine (PG) of Formula (II). In some aspects, the modified internucleotide linkage is a phosphoryl guanidine (PG) of Formula (III). In some aspects, the modified internucleotide linkage is a phosphoryl guanidine (PG) of Formula (IV). In some aspects, the modified internucleotide linkage is a phosphoryl guanidine (PG) of Formula (V).

In some embodiments, the oligonucleotide comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, or more modified internucleotide linkages of Formula (II), Formula (III), Formula (IV), Or Formula (V). In some cases, the oligonucleotide comprises at least about 1, about 2, about 3, about 4, about 5, about 6, about 7, about 8, about 9, about 10, about 11, about 12, about 13, about 14, about 15, or more modified internucleotide linkages of Formula (II), Formula (III), Formula (IV), or Formula (V). In some cases, the oligonucleotide comprises at most about 1, about 2, about 3, about 4, about 5, about 6, about 7, about 8, about 9, about 10, about 11, about 12, about 13, about 14, about 15, or more modified internucleotide linkages of Formula (II), Formula (III), Formula (IV), or Formula (V). In some instances, the modified internucleotide linkages of Formula (II), Formula (III), Formula (IV), or Formula (V) are in tandem within the oligonucleotide. In other instances, the modified internucleotide linkages of Formula (II), Formula (III), Formula (IV), or Formula (V) are interspersed within the oligonucleotide, with nucleotides modified by one or more additional modifications.

In some instances, the oligonucleotide comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification, in which the modification is a modified internucleotide linkages of Formula (II), Formula (III), Formula (IV), or Formula (V). For example, where the oligonucleotide has 20 nucleosides, the oligonucleotide having about 60% modification comprises about 12 nucleosides substituted with 12 Formula (II), Formula (III), Formula (IV), or Formula (V) modified internucleotide linkages.

In some aspects, at least one modified internucleotide linkage of Formula (II), (III), (IV), or (V) is located at the 3′ end of the guide strand. In some aspects, at least one modified internucleotide linkage of Formula (II) is located at the 3′ end of the guide strand. In some aspects, at least one modified internucleotide linkage of Formula (III) is located at the 3′ end of the guide strand. In some aspects, at least one modified internucleotide linkage of Formula (IV) is located at the 3′ end of the guide strand. In some aspects, at least one modified internucleotide linkage of Formula (V) is located at the 3′ end of the guide strand.

In some aspects, at least one modified internucleotide linkage of Formula (II), (III), (IV), or (V) is located within 5, within 4, within 3, or within 2 nucleotides located at the 3′ end of the guide strand. In some aspects, at least one modified internucleotide linkage of Formula (II) is located within 5, within 4, within 3, or within 2 nucleotides located at the 3′ end of the guide strand. In some aspects, at least one modified internucleotide linkage of Formula (III) is located within 5, within 4, within 3, or within 2 nucleotides located at the 3′ end of the guide strand. In some aspects, at least one modified internucleotide linkage of Formula (IV) is located within 5, within 4, within 3, or within 2 nucleotides located at the 3′ end of the guide strand. In some aspects, at least one modified internucleotide linkage of Formula (V) is located within 5, within 4, within 3, or within 2 nucleotides located at the 3′ end of the guide strand.

In some aspects, at least one modified internucleotide linkage of Formula (II), (III), (IV), or (V) is located at the 5′ end of the guide strand. In some aspects, at least one modified internucleotide linkage of Formula (II) is located at the 5′ end of the guide strand. In some aspects, at least one modified internucleotide linkage of Formula (III) is located at the 5′ end of the guide strand. In some aspects, at least one modified internucleotide linkage of Formula (IV) is located at the 5′ end of the guide strand. In some aspects, at least one modified internucleotide linkage of Formula (V) is located at the 5′ end of the guide strand.

In some aspects, at least one modified internucleotide linkage of Formula (II), (III), (IV), or (V) is located within 5, within 4, within 3, or within 2 nucleotides located at the 5′ end of the guide strand. In some aspects, at least one modified internucleotide linkage of Formula (II) is located within 5, within 4, within 3, or within 2 nucleotides located at the 5′ end of the guide strand. In some aspects, at least one modified internucleotide linkage of Formula (III) is located within 5, within 4, within 3, or within 2 nucleotides located at the 5′ end of the guide strand. In some aspects, at least one modified internucleotide linkage of Formula (IV) is located within 5, within 4, within 3, or within 2 nucleotides located at the 5′ end of the guide strand. In some aspects, at least one modified internucleotide linkage of Formula (V) is located within 5, within 4, within 3, or within 2 nucleotides located at the 5′ end of the guide strand.

In some aspects, at least one modified internucleotide linkage of Formula (II), (III), (IV), or (V) is located at the 3′ overhang of the double-stranded oligonucleotide. In some aspects, at least one modified internucleotide linkage of Formula (II) is located at the 3′ overhang of the double-stranded oligonucleotide. In some aspects, at least one modified internucleotide linkage of Formula (III) is located at the 3′ overhang of the double-stranded oligonucleotide. In some aspects, at least one modified internucleotide linkage of Formula (IV) is located at the 3′ overhang of the double-stranded oligonucleotide. In some aspects, at least one modified internucleotide linkage of Formula (V) is located at the 3′ overhang of the double-stranded oligonucleotide. In some aspects, at least one modified internucleotide linkage of Formula (II), (III), (IV), or (V) is located at the 3′ overhang of the guide strand of the double stranded oligonucleotide.

In some aspects, at least one modified internucleotide linkage of Formula (II), (III), (IV), or (V) is located at the 5′ overhang of the double-stranded oligonucleotide. In some aspects, at least one modified internucleotide linkage of Formula (II) is located at the 5′ overhang of the double-stranded oligonucleotide. In some aspects, at least one modified internucleotide linkage of Formula (III) is located at the 5′ overhang of the double-stranded oligonucleotide. In some aspects, at least one modified internucleotide linkage of Formula (IV) is located at the 5′ overhang of the double-stranded oligonucleotide. In some aspects, at least one modified internucleotide linkage of Formula (V) is located at the 5′ overhang of the double-stranded oligonucleotide. In some aspects, at least one modified internucleotide linkage of Formula (II), (III), (IV), or (V) is located at the 5′ overhang of the guide strand of the double stranded oligonucleotide.

In some aspects, the guide strand comprises less than 20, 19, 18, 17, 16, 15, 14, 13, 12, 11, 10, 9, 8, 7, 6, 5, 4, 3, 2, or 1 phosphorothioate internucleotide linkages. In some aspects, the guide strand comprises no more than 1, no more than 2, or no more than 3 phosphorothioate internucleotide linkages at either 5′ end or 3′ end. In some aspects, the guide strand comprises no more than 1, no more than 2, no more than 3, or no more than 4 phosphorothioate internucleotide linkages at 5′ and 3′ ends in total. In some aspects, the guide strand comprises no more than 1, no more than 2, no more than 3, or no more than 4 phosphorothioate internucleotide linkages at each of 5′ and 3′ ends.

In some aspects, the guide strand comprises at least about 20, 19, 18, 17, 16, 15, 14, 13, 12, 11, 10, 9, 8, 7, 6, 5, 4, 3, 2, or 1 phosphorothioate internucleotide linkages. In some aspects, the guide strand further comprises at least one phosphorothioate internucleotide linkage. In some aspects, the guide strand further comprises at least two phosphorothioate internucleotide linkages. In some aspects, the guide strand further comprises at least three phosphorothioate internucleotide linkages. In some aspects, the guide strand further comprises at least four phosphorothioate internucleotide linkages. In some aspects, the guide strand further comprises at least five phosphorothioate internucleotide linkages. In some aspects, the guide strand further comprises at least six phosphorothioate internucleotide linkages. In some aspects, the guide strand further comprises at least seven phosphorothioate internucleotide linkages. In some aspects, the guide strand further comprises at least eight phosphorothioate internucleotide linkages. In some aspects, the guide strand further comprises at least nine phosphorothioate internucleotide linkages. In some aspects, the guide strand further comprises at least 10 phosphorothioate internucleotide linkages. In some aspects, the guide strand further comprises at least 11 phosphorothioate internucleotide linkages. In some aspects, the guide strand further comprises at least 12 phosphorothioate internucleotide linkages. In some aspects, the guide strand further comprises at least 13 phosphorothioate internucleotide linkages. In some aspects, the guide strand further comprises at least 14 phosphorothioate internucleotide linkages. In some aspects, the guide strand further comprises at least 15 phosphorothioate internucleotide linkages.

In some aspects, at least one phosphorothioate internucleotide linkage and at least one modified internucleotide linkage comprising the structure of Formula (II), (III), (IV), or (V) are adjacent to one another (e.g., the phosphorothioate internucleotide linkage is located between the second and third nucleotides and the modified internucleotide linkage comprising the structure of Formula (II), (III), (IV), or (V) is located between the third and fourth nucleotides in the strand). In some aspects, at least one phosphorothioate internucleotide linkage and at least one modified internucleotide linkage comprising the structure of Formula (II) are adjacent to one another. In some aspects, at least one phosphorothioate internucleotide linkage and at least one modified internucleotide linkage comprising the structure of Formula (III) are adjacent to one another. In some aspects, at least one phosphorothioate internucleotide linkage and at least one modified internucleotide linkage comprising the structure of Formula (IV) are adjacent to one another. In some aspects, at least one phosphorothioate internucleotide linkage and at least one modified internucleotide linkage comprising the structure of Formula (V) are adjacent to one another.

In some aspects, the passenger strand comprises at least one modified internucleotide linkage of Formula (II), (III), (IV), or (V). In some aspects, the passenger strand comprises at least two modified internucleotide linkages of Formula (II), (III), (IV), or (V). In some aspects, the passenger strand comprises at least three modified internucleotide linkages of Formula (II), (III), (IV), or (V). In some aspects, the passenger strand comprises at least four modified internucleotide linkages of Formula (II), (III), (IV), or (V). In some aspects, the passenger strand comprises at least five modified internucleotide linkages of Formula (II), (III), (IV), or (V). In some aspects, the passenger strand comprises at least six modified internucleotide linkages of Formula (II), (III), (IV), or (V). In some aspects, the passenger strand comprises at least seven modified internucleotide linkages of Formula (II), (III), (IV), or (V). In some aspects, the passenger strand comprises at least eight modified internucleotide linkages of Formula (II), (III), (IV), or (V). In some aspects, the passenger strand comprises at least nine modified internucleotide linkages of Formula (II), (III), (IV), or (V). In some aspects, the passenger strand comprises at least 10 modified internucleotide linkages of Formula (II), (III), (IV), or (V). In some aspects, the passenger strand comprises at least 11 modified internucleotide linkages of Formula (II), (III), (IV), or (V). In some aspects, the passenger strand comprises at least 12 modified internucleotide linkages of Formula (II), (III), (IV), or (V). In some aspects, the passenger strand comprises at least 13 modified internucleotide linkages of Formula (II), (III), (IV), or (V). In some aspects, the passenger strand comprises at least 14 modified internucleotide linkages of Formula (II), (III), (IV), or (V). In some aspects, the passenger strand comprises at least 15 modified internucleotide linkages of Formula (II), (III), (IV), or (V).

In some aspects, the passenger strand has up to 18 internucleotide linkages of Formula (II), (III), (IV), or (V). In some aspects, the passenger strand has up to 17 internucleotide linkages of Formula (II), (III), (IV), or (V). In some aspects, the passenger strand has up to 16 internucleotide linkages of Formula (II), (III), (IV), or (V). In some aspects, the passenger strand has up to 15 internucleotide linkages of Formula (II), (III), (IV), or (V). In some aspects, the passenger strand has up to 14 internucleotide linkages of Formula (II), (III), (IV), or (V). In some aspects, the passenger strand has up to 13 internucleotide linkages of Formula (II), (III), (IV), or (V). In some aspects, the passenger strand has up to 12 internucleotide linkages of Formula (II), (III), (IV), or (V). In some aspects, the passenger strand has up to 11 internucleotide linkages of Formula (II), (III), (IV), or (V). In some aspects, the passenger strand has up to 10 internucleotide linkages of Formula (II), (III), (IV), or (V). In some aspects, the passenger strand has up to nine internucleotide linkages of Formula (II), (III), (IV), or (V). In some aspects, the passenger strand has up to eight internucleotide linkages of Formula (II), (III), (IV), or (V). In some aspects, the passenger strand has up to seven internucleotide linkages of Formula (II), (III), (IV), or (V). In some aspects, the passenger strand has up to six internucleotide linkages of Formula (II), (III), (IV), or (V). In some aspects, the passenger strand has up to five internucleotide linkages of Formula (II), (III), (IV), or (V). In some aspects, the passenger strand has up to four internucleotide linkages of Formula (II), (III), (IV), or (V). In some aspects, the passenger strand has up to three internucleotide linkages of Formula (II), (III), (IV), or (V). In some aspects, the passenger strand has up to two internucleotide linkages of Formula (II), (III), (IV), or (V). In some aspects, the passenger strand has one internucleotide linkage of Formula (II), (III), (IV), or (V).

In some aspects, at least one modified internucleotide linkage of Formula (II), (III), (IV), or (V) is located within 5, within 4, within 3, or within 2 nucleotides located at the 3′ end of the passenger strand. In some aspects, at least one modified internucleotide linkage of Formula (II) is located within 5, within 4, within 3, or within 2 nucleotides located at the 3′ end of the passenger strand. In some aspects, at least one modified internucleotide linkage of Formula (III) is located within 5, within 4, within 3, or within 2 nucleotides located at the 3′ end of the passenger strand. In some aspects, at least one modified internucleotide linkage of Formula (IV) is located within 5, within 4, within 3, or within 2 nucleotides located at the 3′ end of the passenger strand. In some aspects, at least one modified internucleotide linkage of Formula (V) is located within 5, within 4, within 3, or within 2 nucleotides located at the 3′ end of the passenger strand.

In some aspects, at least one modified internucleotide linkage of Formula (II), (III), (IV), or (V) is located within 5, within 4, within 3, or within 2 nucleotides located at the 5′ end of the passenger strand. In some aspects, at least one modified internucleotide linkage of Formula (II) is located within 5, within 4, within 3, or within 2 nucleotides located at the 5′ end of the passenger strand. In some aspects, at least one modified internucleotide linkage of Formula (III) is located within 5, within 4, within 3, or within 2 nucleotides located at the 5′ end of the passenger strand. In some aspects, at least one modified internucleotide linkage of Formula (IV) is located within 5, within 4, within 3, or within 2 nucleotides located at the 5′ end of the passenger strand. In some aspects, at least one modified internucleotide linkage of Formula (V) is located within 5, within 4, within 3, or within 2 nucleotides located at the 5′ end of the passenger strand.

In some aspects, at least one modified internucleotide linkage of Formula (II), (III), (IV), or (V) is located within 4 or within 3 nucleotides located at the 5′ end of the passenger strand and at least one modified internucleotide linkage of Formula (II), (III), (IV), or (V) is located within 4 or within 3 nucleotides located at the 3′ end of the passenger strand. In some aspects, at least two modified internucleotide linkage of Formula (II), (III), (IV), or (V) is located within 4 or within 3 nucleotides located at the 5′ end of the passenger strand and at least two modified internucleotide linkage of Formula (II), (III), (IV), or (V) is located within 4 or within 3 nucleotides located at the 3′ end of the passenger strand.

In some aspects, the passenger strand comprises less than 18, 17, 16, 15, 14, 13, 12, 11, 10, 9, 8, 7, 6, 5, 4, 3, 2, or 1 phosphorothioate internucleotide linkage. In some aspects, the passenger strand comprises no more than 1, no more than 2, or no more than 3 phosphorothioate internucleotide linkages at either 5′ end or 3′ end. In some aspects, the passenger strand comprises no more than 1, no more than 2, no more than 3, or no more than 4 phosphorothioate internucleotide linkages at 5′ and 3′ ends in total. In some aspects, the passenger strand comprises no more than 1, no more than 2, no more than 3, or no more than 4 phosphorothioate internucleotide linkages at each of 5′ and 3′ ends.

In some aspects, at least one modified internucleotide linkage of Formula (II), (III), (IV), or (V) is not located at the cut site of the passenger strand. In some aspects, at least one modified internucleotide linkage of Formula (II) is not located at the cut site of the passenger strand. In some aspects, at least one modified internucleotide linkage of Formula (III) is not located at the cut site of the passenger strand. In some aspects, at least one modified internucleotide linkage of Formula (IV) is not located at the cut site of the passenger strand. In some aspects, at least one modified internucleotide linkage of Formula (V) is not located at the cut site of the passenger strand. As used herein, the cut site refers to a site in the oligonucleotide cleaved by RNA induced silencing complex (RISC) assembly or Ago2.

In some aspects, the internucleotide linkage of Formula (II), (III), (IV), or (V) on the guide strand or passenger strand increases activity of the conjugate of Formula (I). In some aspects, the internucleotide linkage of Formula (II) on the guide strand or passenger strand increases activity of the conjugate of Formula (I). In some aspects, the internucleotide linkage of Formula (III) on the guide strand or passenger strand increases activity of the conjugate of Formula (I). In some aspects, the internucleotide linkage of Formula (IV) on the guide strand or passenger strand increases activity of the conjugate of Formula (I). In some aspects, the internucleotide linkage of Formula (V) on the guide strand or passenger strand increases activity of the conjugate of Formula (I).

In some aspects, the internucleotide linkage of Formula (II), (III), (IV), or (V) on the guide strand or passenger strand of the siRNA increases the stability (e.g., half-life, etc.) of the conjugate of Formula (I). In some aspects, the internucleotide linkage of Formula (II) on the guide strand or passenger strand of the siRNA increases the stability of the conjugate of Formula (I). In some aspects, the internucleotide linkage of Formula (III) on the guide strand or passenger strand of the siRNA increases the stability of the conjugate of Formula (I). In some aspects, the internucleotide linkage of Formula (IV) on the guide strand or passenger strand of the siRNA increases the stability of the conjugate of Formula (I). In some aspects, the internucleotide linkage of Formula (V) on the guide strand or passenger strand of the siRNA increases the stability of the conjugate of Formula (I).

In some aspects, the oligonucleotide comprises at least one internucleotide linkages of Formula (II), (III), (IV), or (V). In some aspects, at least one modified internucleotide linkage of Formula (II), (III), (IV), or (V) is located at an internal position of the guide strand. In some aspects, at least one modified internucleotide linkage of Formula (II) (III), (IV), or (V) is located at an internal position of the passenger strand.

In some aspects, the oligonucleotide comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, or more internucleotide linkages of Formula (II), (III), (IV), or (V). In some cases, the oligonucleotide comprises at least about 1, about 2, about 3, about 4, about 5, about 6, about 7, about 8, about 9, about 10, about 11, about 12, about 13, about 14, about 15, or more internucleotide linkages of Formula (II), (III), (IV), or (V). In some aspects, the oligonucleotide comprises at least about 10, about 11, about 12, about 13, about 14, about 15, about 16, about 17, about 18, about 19, about 20, about 21, about 22, about 23, about 24, about 25, about 30, or more internucleotide linkages of Formula (II), (III), (IV), or (V). In some aspects, the oligonucleotide comprises at least about 15, about 16, about 17, about 18, about 19, about 20, about 21, about 22, about 23, about 24, about 25, about 30, or more internucleotide linkages of Formula (II), (III), (IV), or (V). In some aspects, the oligonucleotide comprises at least about 20, about 21, about 22, about 23, about 24, about 25, about 30, or more internucleotide linkages of Formula (II), (III), (IV), or (V).

In some aspects, the oligonucleotide has at least one internucleotide linking group represented by Formula (II), (III), (IV), or (V). In some aspects, the oligonucleotide has at least two internucleotide linking groups represented by Formula (II), (III), (IV), or (V). In some aspects, the oligonucleotide has at least three internucleotide linking groups represented by Formula (II), (III), (IV), or (V). In some aspects, the oligonucleotide has at least four internucleotide linking groups represented by Formula (II), (III), (IV), or (V). In some aspects, the oligonucleotide has at least five internucleotide linking groups represented by Formula (II), (III), (IV), or (V). In some aspects, the oligonucleotide has at least six internucleotide linking groups represented by Formula (II), (III), (IV), or (V). In some aspects, the oligonucleotide has at least seven internucleotide linking groups represented by Formula (II), (III), (IV), or (V). In some aspects, the oligonucleotide has at least eight internucleotide linking groups represented by Formula (II), (III), (IV), or (V). In some aspects, the oligonucleotide has at least nine internucleotide linking groups represented by Formula (II), (III), (IV), or (V). In some aspects, the oligonucleotide has at least 10 internucleotide linking groups represented by Formula (II), (III), (IV), or (V). In some aspects, the oligonucleotide has at least 11 internucleotide linking groups represented by Formula (II), (III), (IV), or (V). In some aspects, the oligonucleotide has at least 12 internucleotide linking groups represented by Formula (II), (III), (IV), or (V). In some aspects, the oligonucleotide has at least 13 internucleotide linking groups represented by Formula (II), (III), (IV), or (V). In some aspects, the oligonucleotide has at least 14 internucleotide linking groups represented by Formula (II), (III), (IV), or (V). In some aspects, the oligonucleotide has at least 15 internucleotide linking groups represented by Formula (II), (III), (IV), or (V). In some aspects, the oligonucleotide has at least 20 internucleotide linking groups represented by Formula (II), (III), (IV), or (V).

In some aspects, the oligonucleotide comprises at least 1, 2, 3, 4, 5, 6, or 7 internucleotide linking groups represented by Formula (II), (III), (IV), or (V) at the 5′ end or at the first 7-11 nucleotides from the 5′ end of the oligonucleotides, and at least 1, 2, 3, 4, 5, 6, or 7 internucleotide linking groups represented by Formula (II), (III), (IV), or (V) at the 3′ end or at the first 7-11 nucleotides from the 3′ end of the oligonucleotides. In some instances, the oligonucleotide comprises more numbers of internucleotide linking groups represented by Formula (II), (III), (IV), or (V) at the 5′ end than 3′ end of the oligonucleotide. In some instances, the oligonucleotide comprises more numbers of internucleotide linking groups represented by Formula (II), (III), (IV), or (V) at the 3′ end than 5′ end of the oligonucleotide.

In some aspects, the passenger strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end. In some aspects, the passenger strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 5′ end. In some aspects, the passenger strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end and linking the first 1, 2, 3, 4, 5, 6, 7, 8, 9, 10, 11, 12, 13, 14, 15, 16, 17, or 18 nucleotides starting from the 5′ end. In some aspects, the passenger strand comprises a modified internucleotide linkage of Formula (II) linking all nucleotides.

In some aspects, the guide strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end. In some aspects, the guide strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 5′ end. In some aspects, the guide strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end and linking the first 1, 2, 3, 4, 5, 6, 7, 8, 9, 10, 11, 12, 13, 14, 15, 16, 17, or 18 nucleotides starting from the 5′ end. In some aspects, the guide strand comprises a modified internucleotide linkage of Formula (II) linking all nucleotides.

In some aspects, the passenger strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end and linking the first 1, 2, 3, 4, 5, 6, 7, 8, 9, 10, 11, 12, 13, 14, 15, 16, 17, or 18 nucleotides starting from the 5′ end, and the guide strand comprises a modified internucleotide linkage of Formula (II) linking the first 1, 2, 3, 4, 5, 6, 7, 8, 9, 10, 11, 12, 13, 14, 15, 16, 17, or 18 nucleotides starting from the 3′ end and linking the first 1, 2, 3, 4, 5, 6, 7, 8, 9, 10, 11, 12, 13, 14, 15, 16, 17, or 18 nucleotides starting from the 5′ end.

In some cases, at least one monomer subunit of the monomer subunits is represented by Formula (V*). In some cases, at least two monomer subunits of the monomer subunits are represented by Formula (V*). In some cases, at least three monomer subunits of the monomer subunits are represented by Formula (V*). In some cases, at least four monomer subunits of the monomer subunits are represented by Formula (V*). In some cases, at least five monomer subunits of the monomer subunits are represented by Formula (V*). In some cases, at least six monomer subunits of the monomer subunits are represented by Formula (V*). In some cases, at least seven monomer subunits of the monomer subunits are represented by Formula (V*). In some cases, at least eight monomer subunits of the monomer subunits are represented by Formula (V*). In some cases, at least nine monomer subunits of the monomer subunits are represented by Formula (V*). In some cases, at least 10 monomer subunits of the monomer subunits are represented by Formula (V*). In some cases, at least 11 monomer subunits of the monomer subunits are represented by Formula (V*). In some cases, at least 12 monomer subunits of the monomer subunits are represented by Formula (V*). In some cases, at least 13 monomer subunits of the monomer subunits are represented by Formula (V*). In some cases, at least 14 monomer subunits of the monomer subunits are represented by Formula (V*). In some cases, at least 15 monomer subunits of the monomer subunits are represented by Formula (V*). In some cases, at least 20 monomer subunits of the monomer subunits are represented by Formula (V*).

In some instances, the oligonucleotide comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification, in which the modification is an internucleotide linkage of Formula (II), (III), (IV), or (V). For example, where the oligonucleotide has 20 internucleotide linkages, the oligonucleotide having about 60% modification comprises about 12 internucleotide linkages in the oligonucleotide substituted with 12 of Formula (II), (III), (IV), or (V) internucleotide linkages.

In some aspects, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1,1-dimethylbutyl, 2,2-dimethylbutyl, 3.3-dimethylbutyl, and 2-ethylbutyl groups. In some cases, a modified phosphate is a phosphate group as described in U.S. Pat. No. 9,481,905.

In some aspects, a double-stranded oligonucleotide disclosed herein further comprises a modified internucleotide linkage selected from an alkylphosphonate, a triester, and a mesyl phosphoramidiate. In some aspects, a double-stranded oligonucleotide disclosed herein further comprises a modified internucleotide linkage of an alkylphosphonate. In some aspects, a double-stranded oligonucleotide disclosed herein further comprises a modified internucleotide linkage of a triester. In some aspects, a double-stranded oligonucleotide disclosed herein further comprises a modified internucleotide linkage of a mesyl phosphoramidiate.

In some aspects, additional modified phosphate backbones comprise alkylphosphonate, a triester, methylphosphonate, ethylphosphonate, methylthiophosphonate, or methoxyphosphonate.

In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some aspects, additional modified phosphate backbones comprise one of the followings:

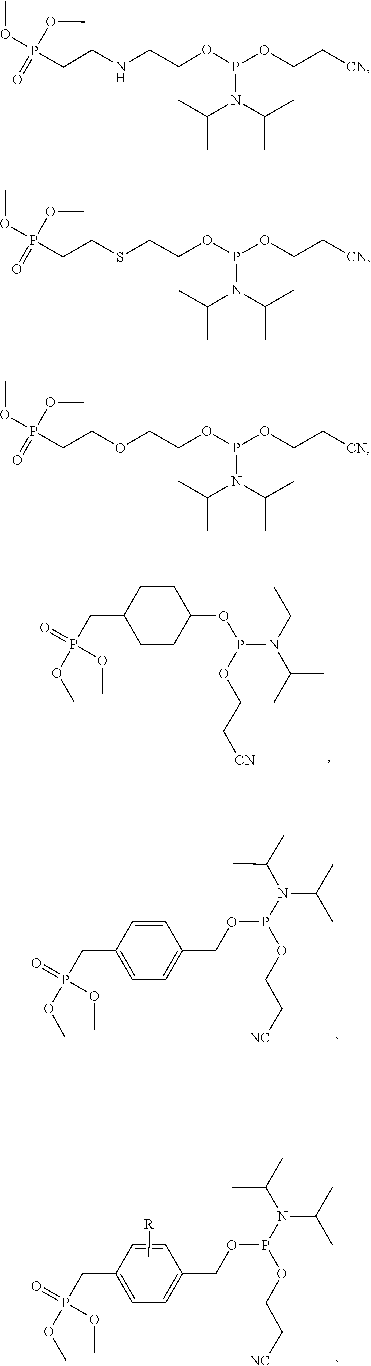

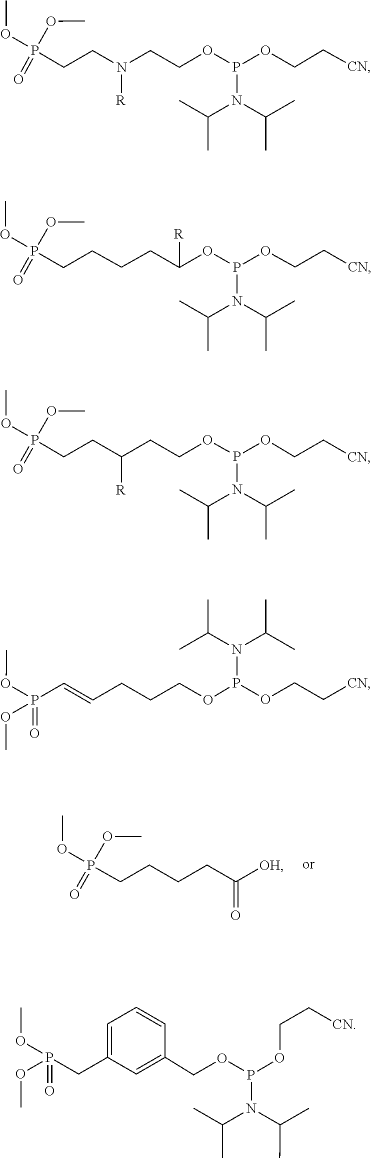

In some instances, the oligonucleotide comprises less than 10, 9, 8, 7, 6, 5, 4, 3, 2, or 1 modified phosphate backbones that are other than Formula (II), (III), (IV), or (V). In some instances, the oligonucleotide comprises less than less than 18, 17, 16, 15, 14, 13, 12, 11, 10, 9, 8, 7, 6, or 5 phosphate backbones that are other than Formula (II), (III), (IV), or (V).

In some aspects, the oligonucleotide is further hybridized with a second oligonucleotide to form a duplex. In some instances, the oligonucleotide is a sense strand or passenger strand. In some instances, the second oligonucleotide is an antisense strand or guide strand. In some aspects, the second oligonucleotide is an RNA oligonucleotide. In some cases, the second oligonucleotide is a modified oligonucleotide. In some cases, the second oligonucleotide has at least one internucleotide linking group as represented by Formula (II), (III), (IV), or (V).

### Modified Ribose

In some aspects, the oligonucleotide comprises synthetic or artificial nucleotide analogues or bases. In some aspects, the oligonucleotide comprises at least one non-natural nucleotide. In some instances, the synthetic or artificial nucleotide analogues or bases comprise a nucleic acid with a modification at a 2′-hydroxyl group of the ribose moiety (2′-modified nucleotide). In some instances, the modification includes an H, OR, R, halo, SH, SR, NH₂, NHR, NR₂, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols, and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some aspects, at least one 2′-modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified nucleotide. In some aspects, the 2′-modified nucleotide comprises 2′-O-methyl modified nucleotide. In some aspects, the 2′-modified nucleotide comprises 2′-O-methoxyethyl (2′-O-MOE) modified nucleotide. In some aspects, the 2′-modified nucleotide comprises 2′-O-aminopropyl modified nucleotide. In some aspects, the 2′-modified nucleotide comprises 2′-deoxy modified nucleotide. In some aspects, the 2′-modified nucleotide comprises 2′-deoxy-2′-fluoro modified nucleotide. In some aspects, the 2′-modified nucleotide comprises 2′-O-aminopropyl (2′-O-AP) modified nucleotide. In some aspects, the 2′-modified nucleotide comprises 2′-O-dimethylaminoethyl (2′-O-DMAOE) modified nucleotide. In some aspects, the 2′-modified nucleotide comprises 2′-O-dimethylaminopropyl (2′-O-DMAP) modified nucleotide. In some aspects, the 2′-modified nucleotide comprises 2′-O-dimethylaminoethyloxyethyl (2′-O-DMAEOE) modified nucleotide. In some aspects, the 2′-modified nucleotide comprises 2′-O—N-methylacetamido (2′-O—NMA) modified nucleotide. In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

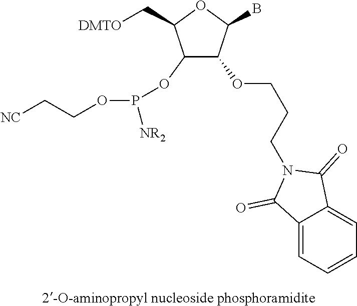

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some aspects, the oligonucleotide described herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more 2′-modified nucleotides.

In some aspects, the oligonucleotide described herein comprises a sense strand (passenger strand) and an antisense strand (guide strand), and the sense strand or the antisense strand comprises at least 1, 2, 3, 4, 5, 6, 7, 8, 9, 10, 11, 12, 13, 14, 15, 16, 17, 18, 19, 20, 21, 22, 23, 24, 25 or more 2′-modified nucleotides. In some aspects, the oligonucleotide described herein comprises a sense strand (passenger strand) and an antisense strand (guide strand), and the sense strand comprises at least 1, 2, 3, 4, 5, 6, 7, 8, 9, 10, 11, 12, 13, 14, 15, 16, 17, 18, 19, 20, 21, 22, 23, 24, 25 or more 2′-modified nucleotides. In some aspects, the oligonucleotide described herein comprises a sense strand (passenger strand) and an antisense strand (guide strand), and the antisense strand comprises at least 1, 2, 3, 4, 5, 6, 7, 8, 9, 10, 11, 12, 13, 14, 15, 16, 17, 18, 19, 20, 21, 22, 23, 24, 25 or more 2′-modified nucleotides. In some aspects, the oligonucleotide described herein comprises a sense strand (passenger strand) and an antisense strand (guide strand), and each of the sense strand and the antisense strand comprises at least 1, 2, 3, 4, 5, 6, 7, 8, 9, 10, 11, 12, 13, 14, 15, 16, 17, 18, 19, 20, 21, 22, 23, 24, 25 or more 2′-modified nucleotides.

In some instances, the 5′-phosphonate modified nucleotide is further modified at the 2′ hydroxyl group in a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer.

Exemplary representations of the chemical structure of 5′-phosphonate modified LNA are illustrated below, wherein J is an internucleotide linkage.

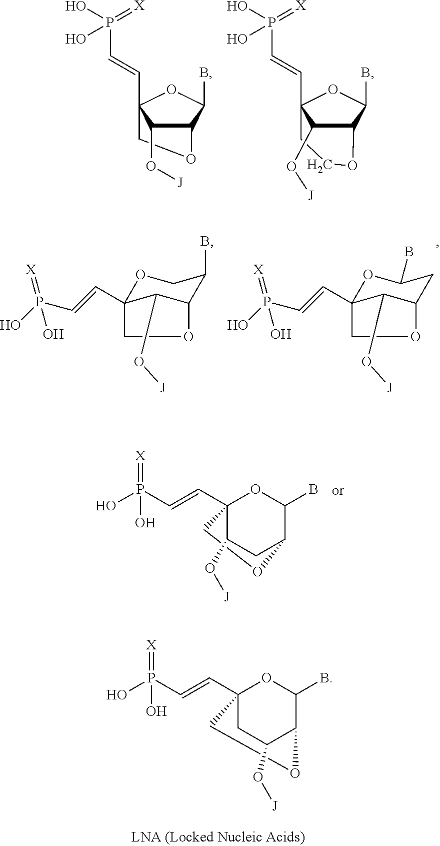

In some aspects, at least one 2′-modified nucleotide comprises ethylene nucleic acid (ENA). ENAs are part of the bridged nucleic acids class of modified nucleic acids that also comprises LNA. Exemplary chemical structures of the ENA and bridged nucleic acids are illustrated below.

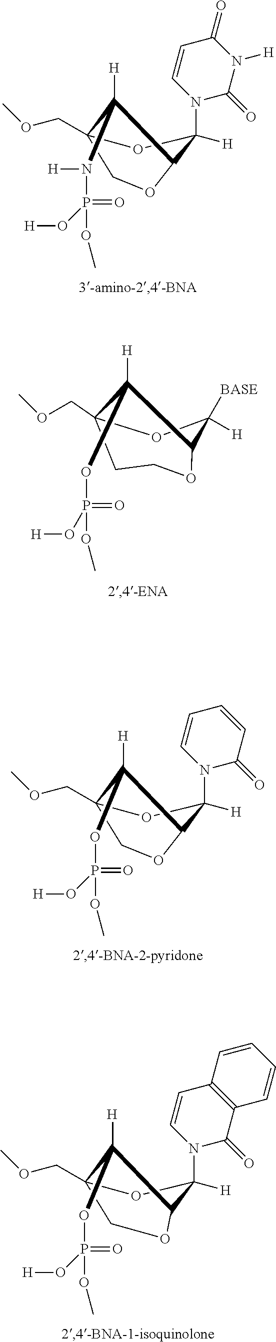

In some aspects, the oligonucleotide described herein comprises at least 1, 2, 3, 4, 5, 6, 7, 8, 9, 10, 11, 12, 13, 14, 15, 16, 17, 18, 19, 20 or more locked nucleic acid (LNA) or ethylene nucleic acid (ENA). In some aspects, the oligonucleotide described herein comprises a sense strand (passenger strand) and an antisense strand (guide strand), and the sense strand or the antisense strand comprises at least 1, 2, 3, 4, 5, 6, 7, 8, 9, 10, or more locked nucleic acid (LNA) or ethylene nucleic acid (ENA). In some aspects, the oligonucleotide described herein comprises a sense strand (passenger strand) and an antisense strand (guide strand), and the sense strand comprises at least 1, 2, 3, 4, 5, 6, 7, 8, 9, 10, or more locked nucleic acid (LNA) or ethylene nucleic acid (ENA). In some aspects, the oligonucleotide described herein comprises a sense strand (passenger strand) and an antisense strand (guide strand), and the antisense strand comprises at least 1, 2, 3, 4, 5, 6, 7, 8, 9, 10, or more locked nucleic acid (LNA) or ethylene nucleic acid (ENA). In some aspects, the oligonucleotide described herein comprises a sense strand (passenger strand) and an antisense strand (guide strand), and each of the sense strand or the antisense strand comprises at least 1, 2, 3, 4, 5, 6, 7, 8, 9, 10, or more locked nucleic acid (LNA) or ethylene nucleic acid (ENA).

In some aspects, nucleotide analogues comprise modified bases (e.g., B, heterocyclic base moiety) such as, but not limited to, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof. Morpholinos or phosphorodiamidate morpholino oligomers (PMOs) comprise synthetic molecules whose structures mimic natural nucleic acid structures by deviating from the normal sugar and phosphate structures. In some instances, the five-membered ribose ring is substituted with a six-membered morpholino ring containing four carbons, one nitrogen and one oxygen. In some cases, the ribose monomers are linked by a phosphorodiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

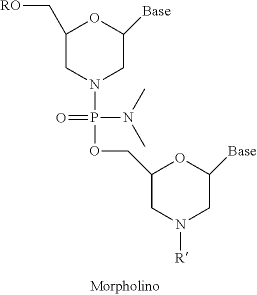

In some aspects, a 5′-phosphonate modified morpholino or PMO described herein is a PMO comprising a positive or cationic charge. In some instances, the PMO is PMOplus (Sarepta). PMOplus refers to phosphorodiamidate morpholino oligomers comprising any number of (1-piperazino)phosphinylideneoxy(1-(4-(omega-guanidino-alkanoyl))-piperazino)phosphinylideneoxy linkages (e.g., as such those described in PCT Publication No. WO2008/036127). In some cases, the PMO is a PMO described in U.S. Pat. No. 7,943,762.

In some aspects, a morpholino or PMO described herein is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some aspects, a morpholino or PMO described herein is a PMO as described in Table 5 of U.S. Publication No. 2014/0296321.

In some aspects, the peptide nucleic acid (PNA) does not contain a sugar ring or phosphate linkage and the bases are attached and appropriately spaced by oligoglycine-like molecules, therefore, eliminating a backbone charge.

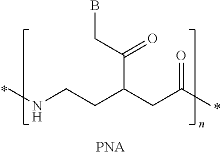

In some aspects, one or more modifications optionally occur at the internucleotide linkage. In some instances, modified internucleotide linkages include, but are not limited to, phosphorothioates, phosphorodithioates, methylphosphonates, 5′-alkylenephosphonates, 5′-methylphosphonates, 3′-alkylene phosphonates, borontrifluoridates, borano phosphate esters and selenophosphates with 3′-5′ linkages or 2′-5′ linkages, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

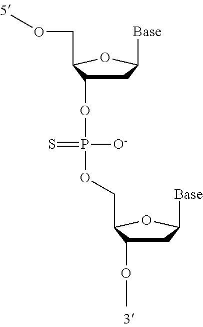

In some instances, the modification is a methyl or thiol modification such as methylphosphonate or thiolphosphonate modification. An exemplary thiolphosphonate nucleotide (left) and an methylphosphonate nucleotide (right) are illustrated below.

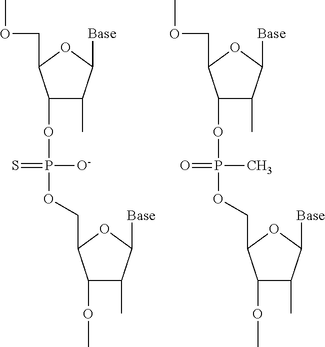

In some instances, a modified nucleotide includes, but is not limited to, 2′-fluoro N3-P5′-phosphoramidites illustrated as:

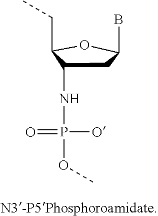

In some instances, a modified nucleotide includes, but is not limited to, hexitol nucleic acid (or 1′,5′-anhydrohexitol nucleic acids (HNA)) illustrated as:

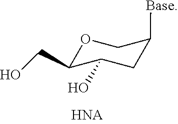

In some aspects, an oligonucleotide comprises a sense strand and antisense strand, and the sense strand or the antisense includes a terminal cap moiety at the 5′-end, the 3′-end, or both of the 5′ and 3′ ends of the sense strand. In other embodiments, the terminal cap moiety is an inverted deoxy abasic moiety. In some aspects, at least one inverted abasic moiety is at least one terminus.

In some aspects, a double-stranded oligonucleotide, and/or oligonucleotide comprising a nucleotide analogue or artificial nucleotide base described above further comprises a 5′ terminal vinylphosphonate modified nucleotide. In some aspects, a nucleotide analogue or artificial nucleotide base described above comprises a 5′-vinylphosphonate modified nucleotide with a modification at a 5′ hydroxyl group of the ribose moiety. In some aspects, the 5′-vinylphosphonate modified nucleotide is selected from the nucleotides provided below, wherein the linker is O or S; and B is a heterocyclic base moiety.

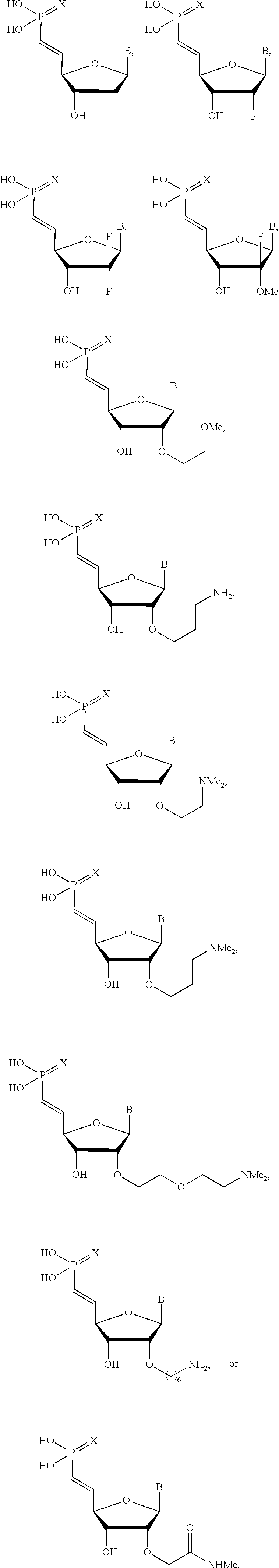

Additional examples of 5′-vinylphosphonate modified nucleic acids are illustrated below, wherein the linker is O or S; B is a heterocyclic base moiety; and J is an internucleotide linkage:

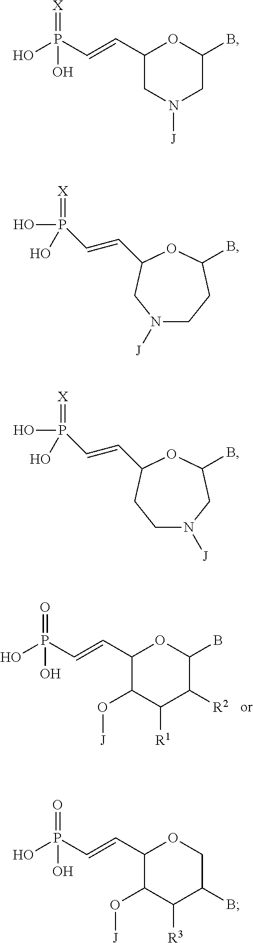

wherein B is a heterocyclic base moiety;
R⁴, and R⁵ are independently selected from hydrogen, halogen, alkyl or alkoxy; and
J is an internucleotide linking group linking to the adjacent nucleotide of the oligonucleotide.

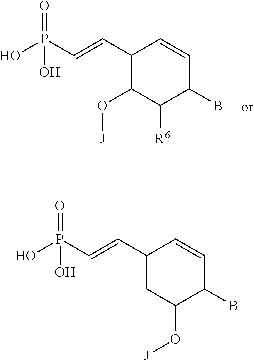

wherein B is a heterocyclic base moiety;
R⁶ is selected from hydrogen, halogen, alkyl or alkoxy; and
J is an internucleotide linking group linking to the adjacent nucleotide of the oligonucleotide.

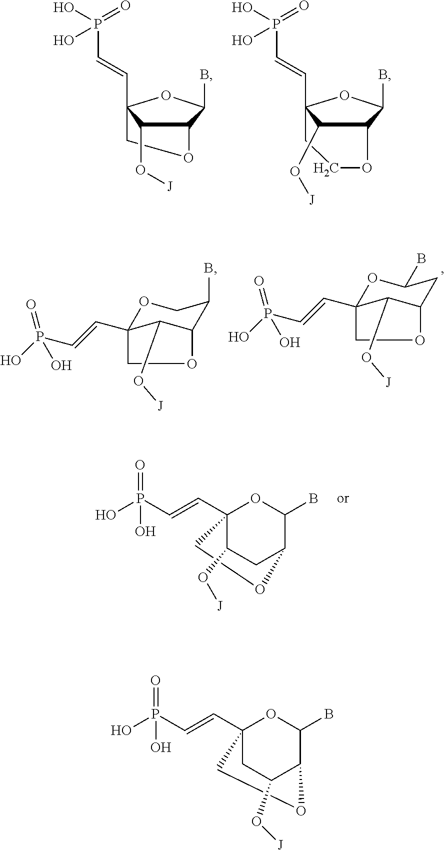

wherein B is a heterocyclic base moiety; and
J is an internucleotide linking group linking to the adjacent nucleotide of the oligonucleotide.

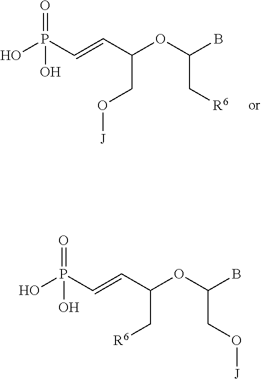

wherein B is a heterocyclic base moiety;
R⁶ is selected from hydrogen, halogen, alkyl or alkoxy; and
J is an internucleotide linking group linking to the adjacent nucleotide of the oligonucleotide.

In some instances, the oligonucleotide comprises a first strand and a second strand. In some aspects, the first strand: is a sense strand; is an RNA oligonucleotide; is conjugated to a binding moiety, a polymer, or a combination thereof; is from about 10 to about 30 nucleotides; comprises at least one internucleotide linking group having Formula (II), (III), (IV), or (V); and comprises at least one monomer subunit of Formula (V*). In some aspects, the second strand: is an antisense strand; is an RNA oligonucleotide; is from about 10 to about 30 nucleotides; comprises at least one internucleotide linking group having Formula (II), (III), (IV), or (V); and comprises at least one monomer subunit of Formula (V*).

In some instances, the oligonucleotide is from about 10 to about 50 nucleotides in length. In some instances, the oligonucleotide is from about 10 to about 45 nucleotides in length. In some instances, the oligonucleotide is from about 10 to about 40 nucleotides in length. In some instances, the oligonucleotide is from about 10 to about 35 nucleotides in length. In some instances, the oligonucleotide is from about 10 to about 30 nucleotides in length. In some instances, the oligonucleotide is from about 10 to about 25 nucleotides in length. In some instances, the oligonucleotide is from about 10 to about 20 nucleotides in length. In some instances, the oligonucleotide is from about 15 to about 25 nucleotides in length. In some instances, the oligonucleotide is from about 19 to about 23 nucleotides in length. In some instances, the oligonucleotide is from about 15 to about 30 nucleotides in length. In some instances, the oligonucleotide is from about 12 to about 30 nucleotides in length. In some aspects, the oligonucleotide is from about 8 to about 50 nucleotides in length.

In some aspects, the oligonucleotide is about 50 nucleotides in length. In some instances, the oligonucleotide is about 45 nucleotides in length. In some instances, the oligonucleotide is about 40 nucleotides in length. In some instances, the oligonucleotide is about 35 nucleotides in length. In some instances, the oligonucleotide is about 30 nucleotides in length. In some instances, the oligonucleotide is about 25 nucleotides in length. In some instances, the oligonucleotide is about 20 nucleotides in length. In some instances, the oligonucleotide is about 19 nucleotides in length. In some instances, the oligonucleotide is about 18 nucleotides in length. In some instances, the oligonucleotide is about 17 nucleotides in length. In some instances, the oligonucleotide is about 16 nucleotides in length. In some instances, the oligonucleotide is about 15 nucleotides in length. In some instances, the oligonucleotide is about 14 nucleotides in length. In some instances, the oligonucleotide is about 13 nucleotides in length. In some instances, the oligonucleotide is about 12 nucleotides in length. In some instances, the oligonucleotide is about 11 nucleotides in length. In some instances, the oligonucleotide is about 10 nucleotides in length.

In some aspects, the oligonucleotide comprises a sense strand (passenger strand) and an antisense strand (guide strand), and at least one of or each of the sense strand (passenger strand) and the antisense strand (guide strand) is from about 10 to about 50 nucleotides in length. In some instances, the at least one of or each of the sense strand (passenger strand) and the antisense strand (guide strand) is from about 10 to about 30, from about 15 to about 30, from about 18 to about 25, from about 18 to about 24, from about 19 to about 23, or from about 20 to about 22 nucleotides in length.

In some instances, at least one of or each of the sense strand (passenger strand) and the antisense strand (guide strand) is from about 10 to about 50, from about 10 to about 45 nucleotides, from about 10 to about 40 nucleotides, from about 10 to about 35 nucleotides, from about 10 to about 30 nucleotides, from about 10 to about 25 nucleotides, from about 10 to about 20 nucleotides, from about 15 to about 25 nucleotides, from about 15 to about 30 nucleotides, from about 19 to about 23 nucleotides, or from about 12 to about 30 nucleotides in length.

In some instances, at least one of or each of the sense strand (passenger strand) and the antisense strand (guide strand) is about 50, 45, 40, 35, 30, 25, or 20 nucleotides in length. In some instances, at least one of or each of the sense strand (passenger strand) and the antisense strand (guide strand) is about 19, 18, 17, 16, 15, 14, 13, 12, 12, or 11 nucleotides in length.

In some instances, the guide strand is about 50 nucleotides in length. In some instances, the guide strand is about 45 nucleotides in length. In some instances, the guide strand is about 40 nucleotides in length. In some instances, the guide strand is about 35 nucleotides in length. In some instances, the guide strand is about 30 nucleotides in length. In some instances, the guide strand is about 25 nucleotides in length. In some instances, the guide strand is about 20 nucleotides in length. In some instances, the guide strand is about 19 nucleotides in length. In some instances, the guide strand is about 18 nucleotides in length. In some instances, the guide strand is about 17 nucleotides in length. In some instances, the guide strand is about 16 nucleotides in length. In some instances, the guide strand is about 15 nucleotides in length. In some instances, the guide strand is about 14 nucleotides in length. In some instances, the guide strand is about 13 nucleotides in length. In some instances, the guide strand is about 12 nucleotides in length. In some instances, the guide strand is about 11 nucleotides in length. In some instances, the guide strand is about 10 nucleotides in length. In some instances, the guide strand is from about 10 to about 50 nucleotides in length. In some instances, the guide strand is from about 10 to about 45 nucleotides in length. In some instances, the guide strand is from about 10 to about 40 nucleotides in length. In some instances, the guide strand is from about 10 to about 35 nucleotides in length. In some instances, the guide strand is from about 10 to about 30 nucleotides in length. In some instances, the guide strand is from about 10 to about 25 nucleotides in length. In some instances, the guide strand is from about 10 to about 20 nucleotides in length. In some instances, the guide strand is from about 15 to about 25 nucleotides in length. In some instances, the guide strand is from about 15 to about 25 nucleotides in length. In some instances, the guide strand is from about 15 to about 30 nucleotides in length. In some instances, the guide strand is from about 12 to about 30 nucleotides in length.

In some instances, the passenger strand is about 50 nucleotides in length. In some instances, the passenger strand is about 45 nucleotides in length. In some instances, the passenger strand is about 40 nucleotides in length. In some instances, the passenger strand is about 35 nucleotides in length. In some instances, the passenger strand is about 30 nucleotides in length. In some instances, the passenger strand is about 25 nucleotides in length. In some instances, the passenger strand is about 20 nucleotides in length. In some instances, the passenger strand is about 19 nucleotides in length. In some instances, the passenger strand is about 18 nucleotides in length. In some instances, the passenger strand is about 17 nucleotides in length. In some instances, the passenger strand is about 16 nucleotides in length. In some instances, the passenger strand is about 15 nucleotides in length. In some instances, the passenger strand is about 14 nucleotides in length. In some instances, the passenger strand is about 13 nucleotides in length. In some instances, the passenger strand is about 12 nucleotides in length. In some instances, the passenger strand is about 11 nucleotides in length. In some instances, the passenger strand is about 10 nucleotides in length. In some instances, the passenger strand is from about 10 to about 50 nucleotides in length. In some instances, the passenger strand is from about 10 to about 45 nucleotides in length. In some instances, the passenger strand is from about 10 to about 40 nucleotides in length. In some instances, the passenger strand is from about 10 to about 35 nucleotides in length. In some instances, the passenger strand is from about 10 to about 30 nucleotides in length. In some instances, the passenger strand is from about 10 to about 25 nucleotides in length. In some instances, the passenger strand is from about 10 to about 20 nucleotides in length. In some instances, the passenger strand is from about 15 to about 25 nucleotides in length. In some instances, the passenger strand is from about 15 to about 25 nucleotides in length. In some instances, the passenger strand is from about 15 to about 30 nucleotides in length. In some instances, the passenger strand is from about 12 to about 30 nucleotides in length.

In some embodiments, the oligonucleotide comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides.

In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some embodiments, the sequence of the oligonucleotide is at least 40%, 50%, 55%, 60%, 65%, 70%, 75%, 80%, 85%, 90%, 95%, 98%, 99%, or 99.5% complementary to a target sequence described herein. Exemplary target sequence includes, but not limited to, any sequences of MSTN gene or its mRNA, SSB gene or its mRNA. In some embodiments, the sequence of the oligonucleotide is at least 50% complementary to a target sequence described herein. In some embodiments, the sequence of the oligonucleotide is at least 60% complementary to a target sequence described herein. In some embodiments, the sequence of the oligonucleotide is at least 70% complementary to a target sequence described herein. In some embodiments, the sequence of the oligonucleotide is at least 80% complementary to a target sequence described herein. In some embodiments, the sequence of the oligonucleotide is at least 90% complementary to a target sequence described herein. In some embodiments, the sequence of the oligonucleotide is at least 95% complementary to a target sequence described herein. In some embodiments, the sequence of the oligonucleotide is at least 99% complementary to a target sequence described herein. In some instances, the sequence of the oligonucleotide is 100% complementary to a target sequence described herein.

In some embodiments, the sequence of the oligonucleotide has 5 or less mismatches to a target sequence described herein. In some embodiments, the sequence of the oligonucleotide has 4 or less mismatches to a target sequence described herein. In some instances, the sequence of the oligonucleotide has 3 or less mismatches to a target sequence described herein. In some cases, the sequence of the oligonucleotide has 2 or less mismatches to a target sequence described herein. In some cases, the sequence of the oligonucleotide has 1 or less mismatches to a target sequence described herein.

In some embodiments, the specificity of the oligonucleotide that hybridizes to a target sequence described herein is a 95%, 98%, 99%, 99.5%, or 100% sequence complementarity of the oligonucleotide to a target sequence. In some instances, the hybridization is a high stringent hybridization condition.

In some embodiments, the oligonucleotide hybridizes to at least 8, 9, 10, 11, 12, 13, 14, 15, 16, 17, 18, 19, 20, or more contiguous bases of a target sequence described herein. In some embodiments, the oligonucleotide hybridizes to at least 8 contiguous bases of a target sequence described herein. In some embodiments, the oligonucleotide hybridizes to at least 9 contiguous bases of a target sequence described herein. In some embodiments, the oligonucleotide hybridizes to at least 10 contiguous bases of a target sequence described herein. In some embodiments, the oligonucleotide hybridizes to at least 11 contiguous bases of a target sequence described herein. In some embodiments, the oligonucleotide hybridizes to at least 12 contiguous bases of a target sequence described herein. In some embodiments, the oligonucleotide hybridizes to at least 13 contiguous bases of a target sequence described herein. In some embodiments, the oligonucleotide hybridizes to at least 14 contiguous bases of a target sequence described herein. In some embodiments, the oligonucleotide hybridizes to at least 15 contiguous bases of a target sequence described herein. In some embodiments, the oligonucleotide hybridizes to at least 16 contiguous bases of a target sequence described herein. In some embodiments, the oligonucleotide hybridizes to at least 17 contiguous bases of a target sequence described herein. In some embodiments, the oligonucleotide hybridizes to at least 18 contiguous bases of a target sequence described herein. In some embodiments, the oligonucleotide hybridizes to at least 19 contiguous bases of a target sequence described herein. In some embodiments, the oligonucleotide hybridizes to at least 20 contiguous bases of a target sequence described herein.

### ADDITIONAL MODIFICATIONS

In some aspects, a nucleotide analogue or artificial nucleotide base described herein comprises a 5′-phosphonate modified nucleotide nucleic acid with a modification at a 5′ hydroxyl group of the ribose moiety. In some aspects, a nucleotide analogue or artificial nucleotide base described herein comprises a 5′-vinylphosphonate modified nucleotide nucleic acid with a modification at a 5′ hydroxyl group of the ribose moiety.

In some instances, the modification is a methyl or thiol modification, such as methylphosphonate or thiolphosphonate modification. Exemplary thiolphosphonate nucleotide (left), phosphorodithioates (center), and methylphosphonate nucleotide (right) are illustrated below.

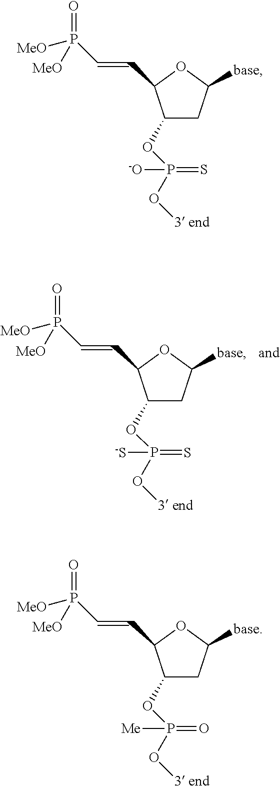

In some instances, a 5′-vinylphosphonate modified nucleotide includes, but is not limited to, phosphoramidites illustrated as:

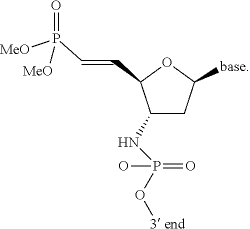

In some instances, the modified internucleotide linkage is a phosphorodiamidate linkage. A non-limiting example of a phosphorodiamidate linkage with a morpholino system is shown below.

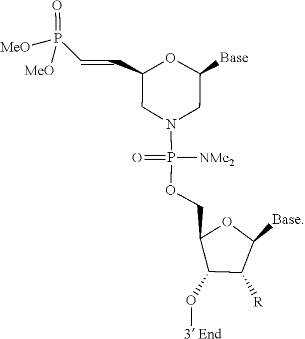

In some instances, the modified internucleotide linkage is a methylphosphonate linkage. A non-limiting example of a methylphosphonate linkage is shown below.

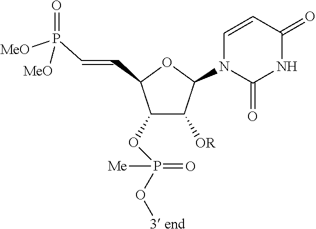

In some instances, the modified internucleotide linkage is an amide linkage. A non-limiting example of an amide linkage is shown below.

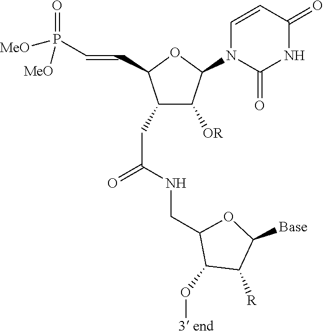

In some aspects, one or more modifications further optionally include modifications of the ribose moiety, phosphate backbone and the nucleotide, or modifications of the nucleotide analogues at the 3′ or the 5′ terminus. For example, the 3′ terminus optionally includes a 3′ cationic group, or by inverting the nucleotide at the 3′-terminus with a 3′-3′ linkage. In another alternative, the 3′-terminus is optionally conjugated with an aminoalkyl group, e.g., a 3′ C₅-aminoalkyl dT. In an additional alternative, the 3′-terminus is optionally conjugated with an abasic site, e.g., with an apurinic or apyrimidinic site.

In some aspects, the oligonucleotide has a NH₂—C₁₋₁₂ alkyl conjugated to the 5′-terminus of the modified oligonucleotide. In some aspects, a NH₂—C₁₋₆ alkyl is conjugated to the 5′-terminus of the oligonucleotide. In some aspects, the modified oligonucleotide has a NH₂—C₆ alkyl conjugated to the 5′-terminus of the modified oligonucleotide.

In some aspects, the oligonucleotide comprising at least one internucleotide linkage represented by Formula (II), (III), (IV), or (V) further comprises one or more of the artificial nucleotide analogues described herein. In some instances, the oligonucleotide comprising at least one internucleotide linkage represented by Formula (II), (III), (IV), or (V) further comprises one or more, e.g., 1, 2, 3, 4, 5, 6, 7, 8, 9, 10, 11, 12, 13, 14, 15, 16, 17, 18, 20, 25, or more additional modifications, such as, but are not limited to, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide comprising at least one internucleotide linkage represented by Formula (II), (III), (IV), or (V) further comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′O-dimethylaminoethyloxyethyl (2′-O-DMAEOE), 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the oligonucleotide further comprises 1, 2, 3, 4, 5, 6, 7, 8, 9, 10, 11, 12, 13, 14, 15, 16, 17, 18, 20, 25, or more of 2′-O-methyl modified nucleotides. In some instances, the oligonucleotide further comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the oligonucleotide comprising at least one internucleotide linkage represented by Formula (II), (III), (IV), or (V) further comprises 1, 2, 3, 4, 5, 6, 7, 8, 9, 10, 11, 12, 13, 14, 15, 16, 17, 18, 20, 25, or more of thiolphosphonate nucleotides.

In some instances, about 5% to about 100% of the oligonucleotide comprises the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, or 100% of the oligonucleotide comprises the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O—NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, the oligonucleotide described herein comprises RNA or DNA. In some cases, the oligonucleotide comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the RNA comprises siRNA. In some cases, the oligonucleotide comprises a sense strand (or passenger strand) of a siRNA. In other cases, the oligonucleotide comprises an antisense (or guide strand) of a siRNA.

In some aspects, the oligonucleotide hybridizes to at least 8, 9, 10, 11, 12, 13, 14, 15, 16, 17, 18, or 19 contiguous bases of a target gene sequence. In some aspects, the oligonucleotide hybridizes to the area of the target gene that is at least 80%, 85%, 90%, 95%, or 99% complementary to the nucleic acid sequence of the oligonucleotide. In some instances, the oligonucleotide comprises a nucleic acid sequence that is at least 14, 15, 16, 17, 18, or 19 consecutive nucleotides that are complementary to a target gene sequence with no more than 1, 2, or 3 mismatches. In some aspects, the target gene is associated with an onset, development, or prognosis of a disease. In some aspects, the target gene is associated with an onset, development, or prognosis of a cancer. In some aspects, the target gene is associated with an onset, development, or prognosis of an immune disorder. In some aspects, the target gene is associated with an onset, development, or prognosis of a muscle atrophy or a muscle dystrophy.

### Binding Moiety

In some aspects, an oligonucleotide comprising the internucleotide linkage with Formula (II), (III), (IV), or (V) is conjugated with a binding moiety to form a conjugate (e.g., a therapeutic oligonucleotide conjugate). In some aspects, the binding moiety is conjugated to a 5′-terminus of the oligonucleotide. In some aspects, the binding moiety is conjugated to a 3′-terminus of the oligonucleotide. In some aspects, the binding moiety is conjugated to a 5′-terminus of the passenger strand of the oligonucleotide. In some aspects, the binding moiety is conjugated to a 3′-terminus of the passenger strand of the oligonucleotide. In some aspects, the binding moiety comprises an antibody or antigen binding fragment thereof. In some aspects, the binding moiety comprises a peptide or a small molecule. In some aspects, the binding moiety comprises a peptide. In some aspects, the binding moiety comprises a small molecule. In some aspects, the binding moiety comprises an aptamer.

In some aspects, a binding moiety disclosed herein binds to a cell surface receptor. In some aspects, an antibody or antigen binding fragment thereof disclosed herein binds to a cell surface receptor.

In some aspects, a binding moiety disclosed herein is selected from the group consisting of a polypeptide, a protein, or an antibody or antigen binding fragment thereof. In some aspects, the binding moiety is a polypeptide. In some aspects, the binding moiety is a protein. In some aspects, the binding moiety is an antibody or antigen binding fragment thereof. In some aspects, the binding moiety is a polypeptide. In some instances, the polypeptide is an antibody or its fragment thereof. In some cases, the fragment is an antigen binding fragment. In some instances, the antibody or antigen binding fragment thereof comprises a humanized antibody or antigen binding fragment thereof, human antibody or antigen binding fragment thereof, anti-murine antibody (e.g., anti-mouse antibody, anti-rat antibody, etc.), anti-human antibody (e.g., anti-human transferrin receptor antibody), murine antibody or antigen binding fragment thereof, chimeric antibody or antigen binding fragment thereof, monoclonal antibody or antigen binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, or a chemically modified derivative thereof.

In some instances, the binding moiety is an antibody or antigen binding fragment thereof. In some instances, the binding moiety is a humanized antibody or antigen binding fragment thereof, murine antibody or antigen binding fragment thereof, chimeric antibody or antigen binding fragment thereof, monoclonal antibody or antigen binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or antigen binding fragment thereof, bispecific antibody or biding fragment thereof, or a chemically modified derivative thereof. In some instances, the binding moiety is a humanized antibody or antigen binding fragment thereof. In some instances, the binding moiety is a murine antibody or antigen binding fragment thereof. In some instances, the binding moiety is a chimeric antibody or antigen binding fragment thereof. In some instances, the binding moiety is a monoclonal antibody or antigen binding fragment thereof. In some instances, the binding moiety is a full-size antibody. In some instances, the binding moiety is a monovalent Fab′. In some instances, the binding moiety is a divalent Fab₂. In some instances, the binding moiety is a single-chain variable fragment (scFv).

In some aspects, the binding moiety is a bispecific antibody or antigen binding fragment thereof. In some instances, the bispecific antibody is a trifunctional antibody or a bispecific mini-antibody. In some cases, the bispecific antibody is a trifunctional antibody. In some instances, the trifunctional antibody is a full-length monoclonal antibody comprising binding sites for two different antigens. Exemplary trifunctional antibodies include catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), and TRBS07 (Ektomab; targets GD2/CD3; Trion Research Gmbh). Additional exemplary trifunctional antibodies include mAb² from F-star Biotechnology Ltd. In some instances, the bispecific antibody is a bispecific trifunctional antibody. In some aspects, the bispecific antibody is a bispecific trifunctional antibody selected from: catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), TRBS07 (Ektomab; targets GD2/CD3; Trion Research Gmbh), or a mAb² from F-star Biotechnology Ltd.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some aspects, the bi-specific T-cell engager is a fusion protein that contains two single-chain variable fragments (scFvs) in which the two scFvs target epitopes of two different antigens. Exemplary bispecific mini-antibodies include, but are not limited to, DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some aspects, the binding moiety is a bispecific mini-antibody. In some instances, the binding moiety is a bispecific Fab₂. In some instances, the binding moiety is a bispecific F(ab)′₃ fragment. In some cases, the binding moiety is a bispecific bis-scFv. In some cases, the binding moiety is a bispecific (scFv)₂. In some aspects, the binding moiety is a bispecific diabody. In some aspects, the binding moiety is a bispecific minibody. In some aspects, the binding moiety is a bispecific triabody. In other aspects, the binding moiety is a bispecific tetrabody. In other aspects, the binding moiety is a bi-specific T-cell engager (BiTE). In additional aspects, the binding moiety is a bispecific mini-antibody selected from: DART (dual-affinity re-targeting platform; MacroGenics), blinatumomab (MT103 or AMG103; which targets CD19/CD3; Micromet), MT111 (targets CEA/CD3; Micromet/Amegen), MT 112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), or domain antibodies (dAbs from Domantis/GSK).

In some aspects, the binding moiety is a trispecific antibody. In some instances, the trispecific antibody comprises F(ab)′₃ fragments or a triabody. In some instances, the binding moiety is a trispecific F(ab)′₃ fragment. In some cases, the binding moiety is a triabody. In some aspects, the binding moiety is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some aspects, the binding moiety is an antibody or antigen binding fragment thereof that recognizes a cell surface protein. In some instances, the cell surface protein is an antigen expressed by a cancerous cell. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Ra, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b), transferrin receptor, tumor-associated glycoprotein 72 (TAG-72), TrpM4 (BR22450, FLJ20041, TRPM4, TRPM4B, transient receptor potential cation channel, subfamily M, member 4), and the like. In some instances, the binding moiety is an α-transferrin receptor antibody or antigen binding fragment thereof. In some instances, the binding moiety is an α-human transferrin receptor antibody. In some instances, the binding moiety is an α-human transferrin receptor antibody as described in PCT/US2019/068078, which is incorporated by reference herein.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some aspects, the antibody or antigen binding fragment thereof comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hu1D10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), fclatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), namatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), zatuximab, and the like.

### Conjugation Chemistry

In some cases, an oligonucleotide is conjugated to a binding moiety. In some instances, the binding moiety comprises amino acids, peptides, polypeptides, proteins, antibodies, antigens, toxins, hormones, lipids, nucleotides, nucleosides, sugars, carbohydrates, polymers, such as poly(ethylene glycol) (PEG) and poly(propylene glycol), as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or antigen binding fragment thereof. In some instances, the oligonucleotide is further conjugated to a polymer, and optionally an endosomolytic moiety. In some instances, the oligonucleotide is further conjugated to a polymer, and optionally an endosomolytic moiety. In some embodiment, the polymer comprises poly(ethylene glycol).

In some aspects, a conjugate disclosed herein has a drug-to-antibody ratio (DAR) of is about 1, 2, 3, 4, 5, 6, 7, 8, 9, 10, 11, or greater. In some aspects, the average number of drug molecules conjugated to an antibody forms an average ratio. In some instances, the average ratio is referred to as an average DAR, in which the drug as referred to herein is the oligonucleotide. In some aspects, the average number of drug molecules conjugated to an antibody forms an average ratio. In some instances, the average ratio is referred to as an average DAR, in which the drug as referred to herein is the oligonucleotide comprising at least one or more internucleotide linkages with Formula (II), (III), (IV), or (V). In some aspects, the average DAR ratio of the oligonucleotide with the antibody is about 1, 2, 3, 4, 5, 6, 7, 8, 9, 10, 11, 12, 13, 14, 15, 16 or greater. In some aspects, the average DAR ratio of the oligonucleotide with the antibody is from about 1 to 2, from about 1 to 3, from about 1 to 4, from about 1 to 5, from about 1 to 6, from about 1 to 7, or from about 1 to 8. In some aspects, the average DAR ratio of the oligonucleotide with the antibody is from about 2 to 3, from about 2 to 4, from about 2 to 5, from about 2 to 6, from about 2 to 7, or from about 2 to 8. In some aspects, the average DAR ratio of the oligonucleotide with the antibody is about 2, about 3, about 4. In some aspects, the conjugate has a DAR of about 1:1, 2:1, 3:1, 4:1, 5:1, 6:1, 7:1, 8:1, 9:1, 10:1, or 11:1.

In some aspects, an internucleotide linkage of Formula (II), (III), (IV), or (V) on the guide strand or passenger strand disclosed herein increases activity of the conjugate of Formula (I) when DAR of Formula (I) is more than 1 compared to DAR of Formula (I) is 1. In some aspects, the internucleotide linkage of Formula (II) on the guide strand or passenger strand increases activity of the conjugate of Formula (I) when DAR of Formula (I) is more than 1 compared to DAR of Formula (I) is 1. In some aspects, the internucleotide linkage of Formula (III) on the guide strand or passenger strand increases activity of the conjugate of when DAR of Formula (I) is more than 1 compared to DAR of Formula (I) is 1. In some aspects, the internucleotide linkage of Formula (IV) on the guide strand or passenger strand increases activity of the conjugate of Formula (I) when DAR of Formula (I) is more than 1 compared to DAR of Formula (I) is 1. In some aspects, the internucleotide linkage of Formula (V) on the guide strand or passenger strand increases activity of the conjugate of Formula (I) when DAR of Formula (I) is more than 1 compared to DAR of Formula (I) is 1.

In some aspects, an internucleotide linkage of Formula (II), (III), (IV), or (V) on the guide strand or passenger strand disclosed herein increases activity of the conjugate of Formula (I) when average DAR of Formula (I) is about 2, 3, or 4 compared to average DAR of Formula (I) is about 1. In some aspects, the internucleotide linkage of Formula (II) on the guide strand or passenger strand increases activity of the conjugate of Formula (I) when average DAR of Formula (I) is about 2, 3, or 4 compared to average DAR of Formula (I) is about 1. In some aspects, the internucleotide linkage of Formula (III) on the guide strand or passenger strand increases activity of the conjugate of when average DAR of Formula (I) is about 2, 3, or 4 compared to average DAR of Formula (I) is about 1. In some aspects, the internucleotide linkage of Formula (IV) on the guide strand or passenger strand increases activity of the conjugate of Formula (I) when average DAR of Formula (I) is about 2, 3, or 4 compared to average DAR of Formula (I) is about 1. In some aspects, the internucleotide linkage of Formula (V) on the guide strand or passenger strand increases activity of the conjugate of Formula (I) when average DAR of Formula (I) is about 2, 3, or 4 compared to average DAR of Formula (I) is about 1.

In some aspects, an internucleotide linkage of Formula (II), (III), (IV), or (V) on the guide strand or passenger strand disclosed herein increases activity of the conjugate of Formula (I) when average DAR of Formula (I) is about 2 compared to average DAR of Formula (I) is about 1. In some aspects, the internucleotide linkage of Formula (II) on the guide strand or passenger strand increases activity of the conjugate of Formula (I) when average DAR of Formula (I) is about 2 compared to average DAR of Formula (I) is about 1. In some aspects, the internucleotide linkage of Formula (III) on the guide strand or passenger strand increases activity of the conjugate of when average DAR of Formula (I) is about 2 compared to average DAR of Formula (I) is about 1. In some aspects, the internucleotide linkage of Formula (IV) on the guide strand or passenger strand increases activity of the conjugate of Formula (I) when average DAR of Formula (I) is about 2 compared to average DAR of Formula (I) is about 1. In some aspects, the internucleotide linkage of Formula (V) on the guide strand or passenger strand increases activity of the conjugate of Formula (I) when average DAR of Formula (I) is about 2 compared to average DAR of Formula (I) is about 1.

In some aspects, an oligonucleotide is conjugated to the binding moiety by a chemical ligation process. In some instances, the oligonucleotide is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Nat. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some aspects, the oligonucleotide is conjugated to the binding moiety either site-specifically or non-specifically via native ligation chemistry.

In some instances, an oligonucleotide is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with an oligonucleotide containing an aldehyde group. (see Casi et al., “Site-specific traceless coupling of potent cytotoxic drugs to recombinant antibodies for pharmacodelivery,” JACS 134(13): 5887-5892 (2012))

In some instances, an oligonucleotide is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivative conjugating moiety to form an oxime bond. (see Axup et al., “Synthesis of site-specific antibody-drug conjugates using unnatural amino acids,” PNAS 109(40): 16101-16106 (2012))

In some instances, an oligonucleotide is conjugated to the binding moiety by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized oligonucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the oligonucleotide is conjugated to the binding moiety utilizing a microbial transglutaminze catalyzed process. In some instances, mTG catalyzes the formation of a covalent bond between the amide side chain of a glutamine within the recognition sequence and a primary amine of a functionalized oligonucleotide. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the oligonucleotide is conjugated to the binding moiety by a method as described in PCT Publication No. WO2014/140317, which utilizes a sequence-specific transpeptidase. In some instances, the oligonucleotide is conjugated to the binding moiety by a method as described in U.S. Patent Publication Nos. 2015/0105539 and 2015/0105540.

### Linker

In some aspects, a binding moiety and one or more oligonucleotide is conjugated via a bond.

In some aspects, a binding moiety and one or more oligonucleotide is conjugated via a linker. In some aspects, a linker is further conjugated to a conjugate described herein, an oligonucleotide described herein, an oligonucleotide described herein, a binding moiety described herein, or in combinations thereof. In some cases, the linker bridges an oligonucleotide to a binding moiety. In some cases, the linker bridges an oligonucleotide to a polypeptide. In some cases, the linker bridges an oligonucleotide to an antibody or antigen binding fragment thereof.

In some aspects, a linker is a C₁-C₆ alkyl group. In some aspects, the linker is a C₁ alkyl group. In some aspects, the linker is a C₂ alkyl group. In some aspects, the linker is a C₃ alkyl group. In some aspects, the linker is a C₄ alkyl group. In some aspects, the linker is a C₅ alkyl group. In some aspects, the linker is a C₆ alkyl group.

In some aspects, the linker comprises a homobifunctional linker. In some aspects, the linker is a homobifunctional linker, optionally conjugated to a C₁-C₆ alkyl group. In some aspects, the linker is a homobifunctional linker, optionally conjugated to a C₁ alkyl group. In some aspects, the linker is a homobifunctional linker, optionally conjugated to a C₂ alkyl group. In some aspects, the linker is a homobifunctional linker, optionally conjugated to a C₃ alkyl group. In some aspects, the linker is a homobifunctional linker, optionally conjugated to a C₄ alkyl group. In some aspects, the linker is a homobifunctional linker, optionally conjugated to a C₅ alkyl group. In some aspects, the linker is a homobifunctional linker, optionally conjugated to a C₆ alkyl group.

Exemplary homobifunctional linkers further include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some aspects, the linker comprises a heterobifunctional linker. In some aspects, the linker is a heterobifunctional linker, optionally conjugated to a C₁-C₆ alkyl group. In some aspects, the linker is a heterobifunctional linker, optionally conjugated to a C₁ alkyl group. In some aspects, the linker is a heterobifunctional linker, optionally conjugated to a C₂ alkyl group. In some aspects, the linker is a heterobifunctional linker, optionally conjugated to a C₃ alkyl group. In some aspects, the linker is a heterobifunctional linker, optionally conjugated to a C₄ alkyl group. In some aspects, the linker is a heterobifunctional linker, optionally conjugated to a C₅ alkyl group. In some aspects, the linker is a heterobifunctional linker, optionally conjugated to a C₆ alkyl group.

In some aspects, the heterobifunctional linker is succinimidyl-4-(N-maleimidomethyl)cyclohexane-1-carboxylate (sMCC), optionally conjugated to a C₁-C₆ alkyl group. In some aspects, the heterobifunctional linker is succinimidyl-4-(N-maleimidomethyl)cyclohexane-1-carboxylate (sMCC), optionally conjugated to a C₁ alkyl group. In some aspects, the heterobifunctional linker is succinimidyl-4-(N-maleimidomethyl)cyclohexane-1-carboxylate (sMCC), optionally conjugated to a C₂ alkyl group. In some aspects, the heterobifunctional linker is succinimidyl-4-(N-maleimidomethyl)cyclohexane-1-carboxylate (sMCC), optionally conjugated to a C₃ alkyl group. In some aspects, the heterobifunctional linker is succinimidyl-4-(N-maleimidomethyl)cyclohexane-1-carboxylate (sMCC), optionally conjugated to a C₄ alkyl group. In some aspects, the heterobifunctional linker is succinimidyl-4-(N-maleimidomethyl)cyclohexane-1-carboxylate (sMCC), optionally conjugated to a C₅ alkyl group. In some aspects, the heterobifunctional linker is succinimidyl-4-(N-maleimidomethyl)cyclohexane-1-carboxylate (sMCC), optionally conjugated to a C₆ alkyl group.

Exemplary heterobifunctional linker further include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some aspects, a linker described herein is a cleavable linker or a non-cleavable linker. In some instances, the linker is a cleavable linker. In some instances, the linker is an acid cleavable linker. In some instances, the linker is a non-cleavable linker. In some instances, the linker includes a C₁-C₆ alkyl group (e.g., a C₅, C₄, C₃, C₂, or C₁ alkyl group). In some instances, the linker includes homobifunctional cross linkers, heterobifunctional cross linkers, and the like. In some instances, the liker is a traceless linker (or a zero-length linker). In some instances, the linker is a non-polymeric linker. In some cases, the linker is a non-peptide linker or a linker that does not contain an amino acid residue.

In some instances, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described herein.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some aspects, the linker is a dendritic-type linker. In some instances, the dendritic-type linker comprises a branching, multifunctional linker moiety. In some instances, the dendritic-type linker is used to increase the molar ratio of oligonucleotide to the binding moiety. In some instances, the dendritic-type linker comprises PAMAM dendrimers.

In some aspects, the linker is a traceless linker or a linker in which after cleavage does not leave behind a linker moiety (e.g., an atom or a linker group) to a binding moiety, an oligonucleotide, or a conjugate. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker comprises a functional group that exerts steric hinderance at the site of bonding between the linker and a conjugating moiety. In some instances, the steric hinderance is a steric hindrance around a disulfide bond. Exemplary linkers that exhibit steric hinderance comprises a heterobifunctional linker, such as a heterobifunctional linker described herein. In some cases, a linker that exhibits steric hinderance comprises SMCC and SPDB.

In some instances, the linker is an acid cleavable linker. In some instances, the acid cleavable linker comprises a hydrazone linkage, which is susceptible to hydrolytic cleavage. In some cases, the acid cleavable linker comprises a thiomaleamic acid linker. In some cases, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

### Additional Conjugating Moieties

### Polymer Conjugating Moiety

In some aspects, the oligonucleotide comprises a polymer. In some aspects, the polymer comprises poly(ethylene glycol) (PEG). In some aspects, the oligonucleotide comprises poly(ethylene glycol).

In some instances, the polymer moiety is a natural or synthetic polymer, consisting of long chains of branched or unbranched monomers, and/or cross-linked network of monomers in two or three dimensions. In some instances, the polymer moiety includes a polysaccharide, lignin, rubber, or poly(alkylene oxide) (e.g., poly(ethylene glycol)). In some instances, the at least one polymer moiety C includes, but is not limited to, alpha-, omega-dihydroxylpolyethyleneglycol, biodegradable lactone-based polymer, e.g., polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the polymer moiety comprises poly(alkylene oxide). In some instances, the polymer moiety comprises PEG. In some instances, the polymer moiety comprises poly(ethylene imide) (PEI) or hydroxy ethyl starch (HES).

In some instances, the polymer moiety is a PEG moiety. In some instances, the PEG moiety is conjugated at the 5′ terminus of the oligonucleotide while the binding moiety is conjugated at the 3′ terminus of the oligonucleotide. In some instances, the PEG moiety is conjugated at the 3′ terminus of the oligonucleotide while the binding moiety is conjugated at the 5′ terminus of the oligonucleotide. In some instances, the PEG moiety is conjugated to an internal site of the oligonucleotide. In some instances, the PEG moiety, the binding moiety, or a combination thereof, are conjugated to an internal site of the oligonucleotide. In some instances, the conjugation is a direct conjugation. In some instances, the conjugation is via native ligation.

In some instances, the PEG moiety is conjugated at the 5′ terminus of the oligonucleotide while the binding moiety is conjugated at the 3′ terminus of the oligonucleotide. In some instances, the PEG moiety is conjugated at the 3′ terminus of the oligonucleotide while the binding moiety is conjugated at the 5′ terminus of the oligonucleotide. In some instances, the PEG moiety is conjugated to an internal site of the oligonucleotide. In some instances, the PEG moiety, the binding moiety, or a combination thereof, are conjugated to an internal site of the oligonucleotide. In some instances, the conjugation is a direct conjugation. In some instances, the conjugation is via native ligation.

In some aspects, the poly(alkylene oxide) (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some aspects, C is poly- or mono-dispersed poly(alkylene oxide) (e.g., PEG) and the indicated molecular weight represents an average of the molecular weight of the poly(alkylene oxide), e.g., PEG, molecules.

In some aspects, the molecular weight of the poly(alkylene oxide) (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some aspects, the poly(alkylene oxide) (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 44, 46, 48, 50 or more repeating ethylene oxide units. In some cases, a dPEG is synthesized as a single molecular weight compound from pure (e.g., about 95%, 98%, 99%, or 99.5%) staring material in a stepwise fashion. In some cases, a dPEG has a specific molecular weight, rather than an average molecular weight. In some cases, a dPEG described herein is a dPEG from Quanta Biodesign, LMD.

In some instances, cMAP is further conjugated to a PEG moiety, generating a cMAP-PEG copolymer, a mPEG-cMAP-PEGm triblock polymer, or a cMAP-PEG-cMAP triblock polymer. In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

### Endosomolytic or Cell Membrane Penetration Moiety

In some aspects, an oligonucleotide disclosed herein further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof. In some instances, the endosomolytic polypeptide comprises Pep-1 (originated from NLS from Simian Virus 40 large antigen and reverse transcriptase of HIV), Pvec (originated from VE-Cadherin), VT5 (originated from synthetic peptide), C105Y (originated from 1-antitrypsin), transportan (originated from Galanin and mastoparan), TP10 (originated from Galanin and mastoparan), MPG (originated from a hydrophobic domain from the fusion sequence of HIV gp41 and NLS of SV40 T antigen), GH625 (originated from glycoprotein gH of HSV type I), CADY (PPTG1 peptide), GALA (synthetic peptide), INF (Influenza HA2 fusion peptide), HA2E5-TAT (Influenza HA2 subunit of influenza virus X31 strain fusion peptide), HA2-penetratin (Influenza HA2 subunit of influenza virus X31 strain fusion peptide), HA-K4 (Influenza HA2 subunit of influenza virus X31 strain fusion peptide), HA2E4 (Influenza HA2 subunit of influenza virus X31 strain fusion peptide), H5WYG (HA2 analogue), GALA-INF3-(PEG)6-NH (INF3 fusion peptide), or CM18-TAT11 (Cecropin-A-Melittin₂₋₁₂ (CM₁₈) fusion peptide).

In some cases, the endosomolytic moiety comprises a Bak BH3 polypeptide which induces apoptosis through antagonization of suppressor targets such as Bcl-2 and/or Bcl-xL. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

In some aspects, the endosomolytic moiety is a lipid (e.g., a fusogenic lipid). In some aspects, oligonucleotide is further conjugated with an endosomolytic lipid (e.g., fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC). In some instances, an endosomolytic moiety is a lipid (e.g., a fusogenic lipid) described in PCT Publication No. WO09/126,933.

In some aspects, the endosomolytic moiety is a small molecule. In some aspects, an oligonucleotide is further conjugated with an endosomolytic small molecule. Exemplary small molecules suitable as endosomolytic moieties include, but are not limited to, quinine, chloroquine, hydroxychloroquines, amodiaquines (carnoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-)quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha,alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha,alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha,alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof. In some instances, an endosomolytic moiety is a small molecule described in Naisbitt et al (1997, J Pharmacol Exp Therapy 280:884-893) and in U.S. Pat. No. 5,736,557.

In some aspects, cell penetrating polypeptide comprises positively charged short peptides with 5-30 amino acids. In some aspects, cell penetrating polypeptide comprises arginine or lysine rich amino acid sequences. In some aspects, cell penetrating polypeptide includes any polypeptide or combination thereof, including Antennapedia Penetratin (43-58), HIV-1 TAT protein (48-60), pVEC Cadherin (615-632), Transportan Galanine/Mastoparan, MPG HIV-gp41/SV40 T-antigen, Pep-1 HIV-reverse transcriptase/SV40 T-antigen, Polyarginines, MAP, R6W3, NLS, 8-lysines, ARF (1-22), and Azurin-p28.

### Methods

In some aspects, a composition or a pharmaceutical formulation described herein comprising conjugate or an oligonucleotide described herein is used for the treatment of a disease or disorder or for improving the treatment of a disease or disorder by increasing the stability and/or half-life of the oligonucleotide drug molecule.

In another aspect, disclosed herein are methods of treating a subject having a disease or a condition characterized with a defective protein expression and/or a protein overexpression, comprising administering to the subject an oligonucleotide disclosed herein to modulate expression of a gene encoding the protein, thereby treating the disease or condition characterized with the defective protein expression and/or a protein overexpression.

In another aspect, disclosed herein are methods of treating a subject having a disease or a condition characterized with a defective protein expression and/or a protein overexpression, comprising administering to the subject a conjugate disclosed herein to modulate expression of a gene encoding the protein, thereby treating the disease or condition characterized with the defective protein expression and/or a protein overexpression.

In another aspect, disclosed herein are methods of decreasing mRNA levels of a gene in a subject by administering to the subject the conjugate disclosed herein.

In another aspect, disclosed herein are methods of modulating mRNA expression levels of a gene in a subject, the method comprising providing a conjugate described herein and administering to the subject the conjugate, wherein the conjugate decreases mRNA expression levels of the gene in the subject. In some embodiments, the gene is MSTN or SSB.

In another aspect, disclosed herein are methods of treating or myotonic dystrophy in a subject in need thereof, the method comprising providing a conjugate described herein and administering to the subject the conjugate, wherein the conjugate mediates RNA interference against a target mRNA in the subject, thereby treating muscle atrophy or mytonic dystrophy in the subject. In some embodiments, the target mRNA is MSTN mRNA or SSB mRNA.

In some aspects, the conjugate decreases the expression levels of the gene by at least about 20%, 25%, 30%, 35%, 40%, 45%, 50%, 55%, 60%, 65%, 70%, 75%, 80%, 85%, 90%, or 95% relative to a control sample.

In some aspects, the conjugate has an increased plasma half-life relative to an analogous conjugate comprising phosphorothioate internucleotide linkages exchanged at the positions of the modified internucleotide linkages of Formula (II), (III), (IV), or (V).

In another aspect, disclosed herein are methods of increasing siRNA plasma levels in a subject by administering to the subject the conjugate disclosed herein.

In some aspects the disease or the condition is a neuromuscular disease, a muscle dystrophy, a muscle atrophy, a muscle wasting, a genetic disease, cancer, a hereditary disease, or a cardiovascular disease. In some aspects the disease or the condition is a neuromuscular disease. In some aspects the disease or the condition is a muscle dystrophy. In some aspects the disease or the condition is a muscle atrophy. In some aspects the disease or the condition is a muscle wasting. In some aspects the disease or the condition is a genetic disease. In some aspects the disease or the condition is cancer. In some aspects the disease or the condition is a hereditary disease. In some aspects the disease or the condition is a cardiovascular disease. In some aspects, a composition or a pharmaceutical formulation described herein is used as an immunotherapy for the treatment of a disease or disorder. In some instances, the immunotherapy is an immuno-oncology therapy.

In some aspects, the subject is a human.

### Pharmaceutical Formulations

In some aspects, provide herein are pharmaceutical formulations comprising a conjugate or an oligonucleotide disclosed herein for therapeutic applications. In some aspects, provide herein are pharmaceutical formulations comprising a conjugate or an oligonucleotide disclosed herein for treatment of cancer. In some aspects, the pharmaceutical formulations described herein are administered to a subject by multiple administration routes, including, but are not limited to, parenteral (e.g., intravenous, subcutaneous, and intramuscular), oral, intranasal, buccal, rectal, or transdermal administration routes. In some instances, the pharmaceutical composition describe herein is formulated for parenteral (e.g., intravenous, subcutaneous, and intramuscular) administration. In other instances, the pharmaceutical composition describe herein is formulated for oral administration. In still other instances, the pharmaceutical composition describe herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate-release formulations, controlled-release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes, and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of oligonucleotides, with one or more oligonucleotides or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin, dextrin, or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes, or quantum dots. In some instances, a conjugate or an oligonucleotide comprising a binding moiety described herein is conjugated either directly or indirectly to the nanoparticle. In some instances, at least 1, 5, 10, 15, 20, 30, 40, 50, 60, 70, 80, 90, 100, or more conjugates or oligonucleotides comprising binding moieties described herein are conjugated either directly or indirectly to a nanoparticle.

In some aspects, the pharmaceutical formulations include a carrier or carrier materials selected based on compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH-adjusting agents or buffering agents which include acids such as acetic, boric, citric, lactic, phosphoric, and hydrochloric acids; bases, such as sodium hydroxide, sodium phosphate, sodium borate, sodium citrate, sodium acetate, sodium lactate, and tris-hydroxymethylaminomethane; and buffers such as citrate/dextrose, sodium bicarbonate, and ammonium chloride. Such acids, bases, and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but are not limited to, a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid, such as sodium alginate, a clay, such as Veegum® HV (magnesium aluminum silicate), a gum, such as agar, guar, locust bean, Karaya, pectin, or tragacanth, sodium starch glycolate, bentonite, a natural sponge, a surfactant, a resin, such as a cation-exchange resin, citrus pulp, sodium lauryl sulfate, sodium lauryl sulfate in combination starch, and the like.

In some instances, the pharmaceutical formulations include filling agents, such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, poly(ethylene glycol), and the like.

Lubricants and glidants are also optionally included in the pharmaceutical formulations described herein for preventing, reducing, or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil, such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a poly(ethylene glycol) (e.g., PEG-4000) or a methoxypoly(ethylene glycol), such as Carbowax™, sodium oleate, sodium benzoate, glyceryl behenate, poly(ethylene glycol), magnesium or sodium lauryl sulfate, colloidal silica such as Syloid™, Cab-O-Sil*, a starch, such as corn starch, silicone oil, a surfactant, and the like.

Plasticizers include compounds used to soften the microencapsulation material or film coatings to make them less brittle. Suitable plasticizers include, e.g., poly(ethylene glycol)s, such as PEG 300, PEG 400, PEG 600, PEG 1450, PEG 3350, and PEG 800, stearic acid, propylene glycol, oleic acid, triethyl cellulose, and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, poly(ethylene glycol) 200-600, glycofurol, transcutol, propylene glycol, dimethyl isosorbide, and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), poly(ethylene glycol), e.g., the poly(ethylene glycol) can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

Surfactants include compounds, such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

Wetting agents include compounds, such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

### Kits/Article of Manufacture

Disclosed herein, in certain aspects, are kits and articles of manufacture for use with one or more of the compositions and methods described herein. In some aspects, disclosed herein are kits of conjugates disclosed herein. In some aspects, disclosed herein are kits of oligonucleotides disclosed herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a conjugate, as disclosed herein. In another example, the container(s) include an oligonucleotide, as disclosed herein. Such kits optionally include an identifying description or label or instructions relating to its use in the methods described herein.

A kit typically includes labels listing contents and/or instructions for use and package inserts with instructions for use. A set of instructions will also typically be included.

In some instances, a label is on or associated with the container. In one embodiment, a label is on a container when letters, numbers, or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain aspects, the pharmaceutical compositions are presented in a pack or dispenser device which contains one or more unit dosage forms containing a compound provided herein.

The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise.

Furthermore, use of the term “including” as well as other forms, such as “include,” “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s),” “subject(s),” and “patient(s)” mean any mammal. In some aspects, the mammal is a human. In some aspects, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

### Chemical Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or poly-unsaturated and can include mono-, di-, and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted, and each substituent may optionally be different.

The symbol “~~~” denotes the point of attachment of a chemical moiety to the remainder of a molecule or chemical formula.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but are not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, or —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, or —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituent (including, but are not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2- to 20-membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3- to 8-membered heterocycloalkyl, each substituted or unsubstituted aryl is a substituted or unsubstituted C₆-C₁₀ aryl, and each substituted or unsubstituted heteroaryl is a substituted or unsubstituted 5- to 10-membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2- to 8-membered heteroalkyl, each substituted or unsubstituted cycloalkyl is a substituted or unsubstituted C₃-C₇ cycloalkyl, each substituted or unsubstituted heterocycloalkyl is a substituted or unsubstituted -3 to 7-membered heterocycloalkyl, each substituted or unsubstituted aryl is a substituted or unsubstituted C₆-C₁₀ aryl, and each substituted or unsubstituted heteroaryl is a substituted or unsubstituted 5- to 9-membered heteroaryl.

In some certain aspects, each substituted group described in the compounds herein is substituted with at least one substituent group. More specifically, in some certain aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other certain aspects, at least one or all of these groups are substituted with at least one size-limited substituent group. In other certain aspects, at least one or all of these groups are substituted with at least one lower substituent group.

In other aspects of the compounds herein, each substituted or unsubstituted alkyl may be a substituted or unsubstituted C₁-C₂₀ alkyl, each substituted or unsubstituted heteroalkyl is a substituted or unsubstituted 2- to 20-membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3- to 8-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 10-membered heteroaryl. In some aspects of the compounds herein, each substituted or unsubstituted alkylene is a substituted or unsubstituted C₁-C₂₀ alkylene, each substituted or unsubstituted heteroalkylene is a substituted or unsubstituted 2- to 20-membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3- to 8-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 10-membered heteroarylene.

In some certain aspects, each substituted or unsubstituted alkyl is a substituted or unsubstituted C₁-C₈ alkyl, each substituted or unsubstituted heteroalkyl is a substituted or unsubstituted 2- to 8-membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3- to 7-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 9-membered heteroaryl. In some certain aspects, each substituted or unsubstituted alkylene is a substituted or unsubstituted C₁-C₈ alkylene, each substituted or unsubstituted heteroalkylene is a substituted or unsubstituted 2- to 8-membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3- to 7-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 9 membered heteroarylene. In some aspects, the compound is a chemical species set forth in the Examples section, figures, or tables below.

As used herein, the term “isomers” refers to compounds having the same number and kind of atoms, and hence the same molecular weight, but differing in respect to the structural arrangement or configuration of the atoms.

Unless otherwise stated, structures depicted herein are also meant to include all stereochemical forms of the structure. i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2- to 20-membered heteroalkyl,” the group may contain one or more unsubstituted C₁-C₂₀ alkyls, and/or one or more unsubstituted 2- to 20-membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (II), (III), (IV), or (V)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As defined herein, the term “activation,” “activate,” “activating,” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “disease” or “condition” refer to a state of being or health status of a patient or subject capable of being treated with the compounds or methods provided herein. The disease may be a cancer.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, may include prevention of an injury, pathology, condition, or disease. In certain aspects, treating is preventing. In certain aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In certain aspects, prevent refers to slowing the progression of the disease, disorder or condition or inhibiting progression thereof to a harmful or otherwise undesired state.

### EMBODIMENTS

Embodiment 1. A conjugate comprising a binding moiety and an oligonucleotide having at least one internucleotide linkage represented by Formula (V):

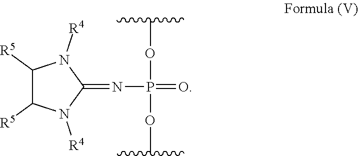

wherein;
   each R⁴ is independently selected from hydrogen or C₁₋₁₀ alkyl; and
   each R⁵ is independently selected from hydrogen, halogen, hydroxy, alkoxy, or C₁₋₁₀ alkyl.
Embodiment 2. The conjugate of embodiment 1, wherein the oligonucleotide is an RNA oligonucleotide.
Embodiment 3. The conjugate of embodiment 1 or 2, further comprising at least one modification.
Embodiment 4. The conjugate of any one of embodiments 1-3, further comprising at least one 2′-modified nucleotide.
Embodiment 5. The conjugate of any one of embodiments 1-4, further comprising at least one 2′-modified nucleotide selected from 2′-O-methyl, 2′-O-methoxyethyl (2′-O-MOE), 2′-deoxy, 2-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified nucleotide.
Embodiment 6. The conjugate of any one of embodiments 1-5, further comprising at least one 2′-modified nucleotide selected from locked nucleic acid (LNA) or ethylene nucleic acid (ENA).
Embodiment 7. The conjugate of any one of embodiments 1-6, wherein the oligonucleotide is conjugated to the binding moiety.
Embodiment 8. The conjugate of any one of embodiments 1-7, wherein the binding moiety is conjugated to the 3-terminus of the oligonucleotide.
Embodiment 9. The conjugate of any one of embodiments 1-8, wherein the binding moiety comprises an antibody or antigen binding fragment thereof.
Embodiment 10. The conjugate of embodiment 9, wherein the antibody or antigen binding fragment thereof comprises a humanized antibody or antigen binding fragment thereof, a chimeric antibody or antigen binding fragment thereof, a monoclonal antibody or antigen binding fragment thereof, a monovalent Fab′, a divalent Fab2, a single-chain variable fragment (scFv), a diabody, a minibody, a nanobody, a single-domain antibody (sdAb), or a camelid antibody or antigen binding fragment thereof.
Embodiment 11. The conjugate of any one of embodiments 1-8, wherein the binding moiety comprises a peptide or a small molecule.
Embodiment 12. The conjugate of any one of embodiments 1-8, wherein the binding moiety comprises an aptamer.
Embodiment 13. The conjugate of any one of embodiments 1-12, comprising from about 8 to about 50 nucleotides.
Embodiment 14. The conjugate of any one of embodiments 1-13, comprising from about 10 to about 30 nucleotides.
Embodiment 15. The conjugate of any one of embodiments 1-14, comprising from about 15 to about 25 nucleotides.
Embodiment 16. The conjugate of any one of embodiments 1-15, comprising 10 nucleotides.
Embodiment 17. The conjugate of any one of embodiments 1-16, wherein each internucleotide linkage is represented by Formula (V).
Embodiment 18. The conjugate of embodiment 1, wherein a NH₂—C₁₋₆ alkyl is conjugated to a 5′-terminus of the oligonucleotide.
Embodiment 19. The conjugate of any one of embodiments 1-18, wherein the oligonucleotide: is an RNA oligonucleotide; is conjugated to the binding moiety; is from about 10 to about 30 nucleotides; and comprises at least one 2′-modified nucleotide.
Embodiment 20. The conjugate of any one of embodiments 1-19, wherein the oligonucleotide hybridizes to at least 8 contiguous bases of a target gene sequence.
Embodiment 21. The conjugate of any one of embodiments 1-20, wherein the oligonucleotide mediates RNA interference.
Embodiment 22. The conjugate of any one of embodiments 1-21, wherein the oligonucleotide is a sense strand.
Embodiment 23. The conjugate of embodiment 22, wherein the oligonucleotide is hybridized with a second oligonucleotide to form a double-stranded polynucleic acid molecule.
Embodiment 24. The conjugate of embodiment 23, wherein the second oligonucleotide is an antisense strand.
Embodiment 25. The conjugate of embodiment 23 or 24, wherein the second oligonucleotide is an RNA oligonucleotide.
Embodiment 26. The conjugate of any one of embodiments 23-25, wherein the second oligonucleotide comprises at least one modification.
Embodiment 27. The conjugate of embodiment 26, wherein the second oligonucleotide comprises at least one 2′-modified nucleotide.
Embodiment 28. The conjugate of embodiment 26 or 27, wherein the second oligonucleotide comprises at least one 2′-modified nucleotide selected from 2′-O-methyl, 2′-0-methoxyethyl (2′-O-MOE), 2′-deoxy, 2-deoxy-2′-fluoro, 2′-O-aminopropyl (2′-O-AP), 2′-O-dimethylaminoethyl (2′-O-DMAOE), 2-O-dimethylaminopropyl (2′-O-DMAP), 2′-O-dimethylaminoethyloxyethyl (2′-O-DMAEOE), or 2′-O—N-methylacetamido (2′-O—NMA) modified nucleotide.
Embodiment 29. The conjugate of any one of embodiments 26-28, wherein the second oligonucleotide comprises at least one 2′-modified nucleotide selected from locked nucleic acid (LNA) or ethylene nucleic acid (ENA).
Embodiment 30. The conjugate of any one of embodiments 1-19 or 22-29, wherein the oligonucleotide comprises a polymer.
Embodiment 31. The conjugate of embodiment 30, wherein the oligonucleotide comprises poly(ethylene glycol).
Embodiment 32. The conjugate of any one of embodiments 1-19 or 22-31, wherein the oligonucleotide comprises a first strand and a second strand, wherein the first strand: is a sense strand; is an RNA oligonucleotide; is conjugated to a binding moiety, a polymer, or a combination thereof; is from about 10 to about 30 nucleotides; comprises at least one 2′-modified nucleotide; and wherein the second strand: is an antisense strand; is an RNA oligonucleotide; is from about 10 to about 30 nucleotides; comprises at least one 2′-modified nucleotide; and comprises at least one modified internucleotide linkage.
Embodiment 33. A method of treating a subject having a disease or a condition characterized with a defective protein expression, comprising administering to the subject a conjugate of any one of embodiments 1-32 to modulate expression of a gene encoding the protein, thereby treating the disease or condition characterized with the defective protein expression.
Embodiment 34. A method of treating a subject having a disease or a condition characterized with a protein overexpression, comprising administering to the subject a conjugate of any one of embodiments 1-32 to modulate expression of a gene encoding the protein, thereby treating the disease or condition characterized with the protein overexpression.
Embodiment 35. The method of embodiment 33 or 34, wherein the disease or the condition is a cancer.
Embodiment 36. The method of embodiment 33 or 34, wherein the disease or the condition is a neuromuscular disease, a muscle dystrophy, a muscle atrophy, a muscle wasting, a genetic disease, cancer, a hereditary disease, or a cardiovascular disease.
Embodiment 37. The method of any one of embodiments 33-36, wherein the subject is a human.
Embodiment 38. A kit comprising the conjugate of any one of embodiments 1 to 32.
Embodiment 39. A modified oligomeric compound comprising a contiguous sequence of monomer subunits linked by internucleotide linking groups wherein at least one of the internucleotide linking groups is represented by Formula (V):

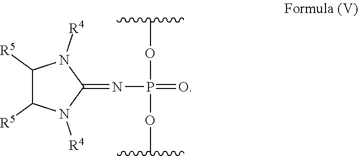

wherein,
   each R⁴ is independently selected from hydrogen or C₁₋₁₀ alkyl; and
   each R⁵ is independently selected from hydrogen, halogen, hydroxy, alkoxy, or C₁₋₁₀ alkyl.
Embodiment 40. The modified oligomeric compound of embodiment 39, wherein at least one monomer subunit of the monomer subunits is represented by Formula (V*):

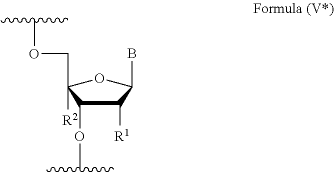

wherein,
   B is independently selected from a heterocyclic base moiety;
   R¹ is independently selected from hydrogen, hydroxy, halogen, or alkoxy; and
   R¹ and R² optionally come together with the atoms to which they are bound to form a C₃-C₄ carbocycle.
Embodiment 41. The modified oligomeric compound of embodiment 39 or 40, wherein at least two monomer subunits of the contiguous sequence are linked by Formula (V) as represented by Formula (VI):

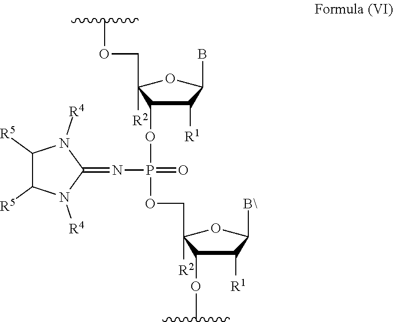

wherein;
   each B is independently selected from a heterocyclic base moiety;
   each R¹ is independently selected from hydrogen, hydroxy, halogen, and alkoxy;
   each R² is independently selected from hydrogen;
   R¹ and R² optionally come together with the atoms to which they are bound to form a C₃-C₄ carbocycle;
   each R⁴ is independently selected from hydrogen or C₁₋₁₀ alkyl; and
   each R⁵ is independently selected from hydrogen, halogen, hydroxy, alkoxy, or C₁₋₁₀ alkyl.
Embodiment 42. The modified oligomeric compound of any one of embodiments 39-41, wherein the modified oligomeric compound has at least two internucleotide linking groups represented by Formula (V).
Embodiment 43. The modified oligomeric compound of any one of embodiments 39-42, wherein the modified oligomeric compound has at least ten internucleotide linking groups represented by Formula (V).
Embodiment 44. The modified oligomeric compound of any one of embodiments 39-43, wherein at least two monomer subunits of the monomer subunits are represented by Formula (V*).
Embodiment 45. The modified oligomeric compound of any one of embodiments 39-44, wherein at least ten monomer subunits of the monomer subunits are represented by Formula (V*).
Embodiment 46. The modified oligomeric compound of any one of embodiments 39-45, wherein the modified oligomeric compound is conjugated to a binding moiety.
Embodiment 47. The modified oligomeric compound of embodiment 46, wherein the binding moiety is conjugated to the 3-terminus of the oligonucleotide.
Embodiment 48. The modified oligomeric compound of embodiment 46 or 47, wherein the binding moiety comprises an antibody or antigen binding fragment thereof.
Embodiment 49. The modified oligomeric compound of embodiment 48, wherein the antibody or antigen binding fragment thereof comprises a humanized antibody or antigen binding fragment thereof, a chimeric antibody or antigen binding fragment thereof, a monoclonal antibody or antigen binding fragment thereof, a monovalent Fab′, a divalent Fab2, a single-chain variable fragment (scFv), a diabody, a minibody, a nanobody, a single-domain antibody (sdAb), or a camelid antibody or antigen binding fragment thereof.
Embodiment 50. The modified oligomeric compound of embodiment 46 or 47, wherein the binding moiety comprises a peptide or a small molecule.
Embodiment 51. The modified oligomeric compound of embodiment 46 or 47, wherein the binding moiety comprises an aptamer.
Embodiment 52. The modified oligomeric compound of any one of embodiments 39-51, comprising from about 8 to about 50 nucleotides.
Embodiment 53. The modified oligomeric compound of any one of embodiments 39-52, comprising from about 10 to about 30 nucleotides.
Embodiment 54. The modified oligomeric compound of any one of embodiments 39-53, comprising from about 15 to about 25 nucleotides.
Embodiment 55. The modified oligomeric compound of any one of embodiments 39-54, comprising 20 nucleotides.
Embodiment 56. The modified oligomeric compound of any one of embodiments 39-55, wherein each internucleotide linkage is represented by Formula (V).
Embodiment 57. The modified oligomeric compound of any one of embodiments 39-56, wherein a NH₂—C₁₋₆ alkyl is conjugated to the 5′-terminus of the modified oligomeric compound.
Embodiment 58. The modified oligomeric compound of any one of embodiments 39-57, wherein the modified oligomeric compound hybridizes to at least 8 contiguous bases of a target gene sequence.
Embodiment 59. The modified oligomeric compound of any one of embodiments 39-58, wherein the modified oligomeric compound mediates RNA interference.
Embodiment 60. The modified oligomeric compound of any one of embodiments 39-59, wherein the modified oligomeric compound is a sense strand.
Embodiment 61. The modified oligomeric compound any one of embodiments 39-57, wherein the modified oligomeric compound is hybridized with a second modified oligomeric compound to form a double-stranded polynucleic acid molecule.
Embodiment 62. The modified oligomeric compound of embodiment 61, wherein the second modified oligomeric compound is an RNA oligonucleotide.
Embodiment 63. The modified oligomeric compound of embodiment 61 or 62, wherein the second modified oligomeric compound is an antisense strand.
Embodiment 64. The modified oligomeric compound of embodiment 61 or 62, wherein the second oligonucleotide comprises a contiguous sequence of monomer subunits linked by internucleotide linking groups wherein at least one of the internucleotide linking groups is represented by Formula (V):

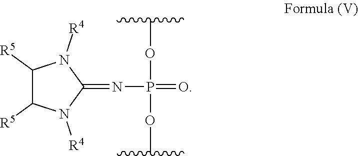

wherein,
   each R⁴ is independently selected from hydrogen or C₁₋₁₀ alkyl; and
   each R⁵ is independently selected from hydrogen, halogen, hydroxy, alkoxy, or C₁₋₁₀ alkyl.
Embodiment 65. The modified oligomeric compound of embodiment 64, wherein at least one monomer subunit of the monomer subunits is represented by Formula (V*):

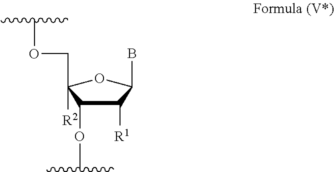

wherein,
   B is independently selected from a heterocyclic base moiety;
   R¹ is independently selected from hydrogen, hydroxy, halogen, or alkoxy;
   R² is independently selected from hydrogen; and
   R¹ and R² optionally come together with the atoms to which they are bound to form a C₃-C₄ carbocycle.
Embodiment 66. The modified oligomeric compound of embodiment 65, wherein at least two monomer subunits of the contiguous sequence are linked by Formula (V) as represented by Formula (V*):

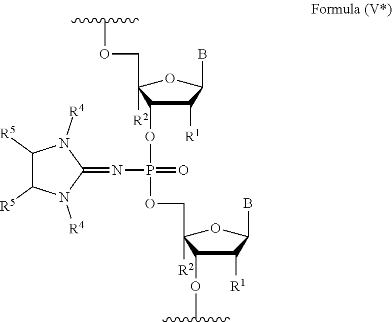

wherein,
   each B is independently selected from a heterocyclic base moiety;
   each R¹ is independently selected from hydrogen, hydroxy, halogen, or alkoxy;
   each R² is independently selected from hydrogen;
   each R¹ and R² optionally come together with the atoms to which they are bound to form a C₃-C₄ carbocycle;
   each R⁴ is independently selected from hydrogen or C₁₋₁₀ alkyl; and
   each R⁵ is independently selected from hydrogen, halogen, hydroxy, alkoxy, or C₁₋₁₀ alkyl.
Embodiment 67. The modified oligomeric compound of any one of embodiments 39-66, wherein the modified oligomeric compound comprises a polymer.
Embodiment 68. The modified oligomeric compound of any one of embodiments 39-67, wherein the modified oligomeric compound comprises poly(ethylene glycol).
Embodiment 69. The modified oligomeric compound of embodiment 39, wherein the modified oligomeric compound comprises a first strand and a second strand, wherein the first strand: is a sense strand; is an RNA oligonucleotide; is conjugated to a binding moiety, a polymer, or a combination thereof; is from about 10 to about 30 nucleotides; comprises at least one internucleotide linking group having Formula (V); comprises at least one monomer subunit of Formula (V*); and wherein the second strand: is an antisense strand; is an RNA oligonucleotide; is from about 10 to about 30 nucleotides; comprises at least one internucleotide linking group having Formula (V); and comprises at least one monomer subunit of Formula (V*).
Embodiment 70. A method of treating a subject having a disease or a condition characterized with a defective protein expression, comprising administering to the subject a modified oligomeric compound of any one of embodiments 39-69 to modulate expression of a gene encoding the protein, thereby treating the disease or condition characterized with the defective protein expression.
Embodiment 71. A method of treating a subject having a disease or a condition characterized with a protein overexpression, comprising administering to the subject a modified oligomeric compound of any one of embodiments 39-69 to modulate expression of a gene encoding the protein, thereby treating the disease or condition characterized with the protein overexpression.
Embodiment 72. The method of embodiment 70 or 71, wherein the disease or the condition is a cancer.
Embodiment 73. The method of embodiment 70 or 71, wherein the disease or the condition is a neuromuscular disease, a muscle dystrophy, a muscle atrophy, a muscle wasting, a genetic disease, cancer, a hereditary disease, or a cardiovascular disease.
Embodiment 74. The method of any one of embodiments 70-73, wherein the subject is a human.
Embodiment 75. A kit comprising a modified oligomeric compound of any one of embodiments 39-74.
Embodiment 76. An oligonucleotide comprising at least one internucleotide linkage represented by Formula (V):

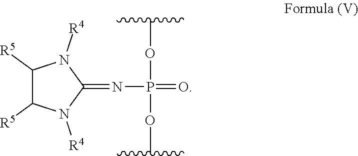

wherein each R⁴ is independently selected from hydrogen or C₁₋₁₀ alkyl; and
wherein said oligonucleotide comprises at least 12 nucleotides.
Embodiment 77. A double-stranded oligonucleotide comprising a guide strand and a passenger strand,
wherein the guide strand or the passenger strand comprises at least one modified internucleotide linkage comprising the structure of Formula (II):

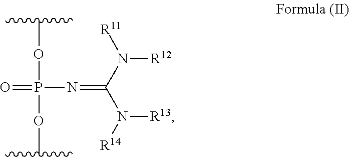

wherein each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl;
optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine.
Embodiment 78. The double-stranded oligonucleotide of embodiment 77, wherein R¹² and R¹³, together with the atom to which they are bound, form an imidazolidine.
Embodiment 79. The double-stranded oligonucleotide of embodiment 77 or 78, wherein the internucleotide linkage has the structure of Formula (III):

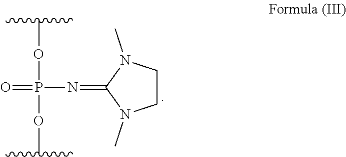 [SMILES: *OP(=O)(N=C1N(C)CCN1C)O*]

Embodiment 80. The double-stranded oligonucleotide of embodiment 77, wherein R¹¹, R¹², R¹³, and R¹⁴ are —C₁₋₁₀ alkyl.
Embodiment 81. The double-stranded oligonucleotide of embodiment 78, wherein the internucleotide linkage has the structure of Formula (IV):

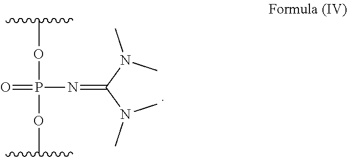 [SMILES: *OP(=O)(N=C(N(C)C)N(C)C)O*]

Embodiment 82. The double-stranded oligonucleotide of any one of embodiments 77-81, wherein the at least one modified internucleotide linkage of Formula (II) is located at the 5′ or 3′ end of the guide strand.
Embodiment 83. The double-stranded oligonucleotide of any one of embodiments 77-81, wherein the at least one modified internucleotide linkage of Formula (II) is located at an internal position of the guide strand.
Embodiment 84. The double-stranded oligonucleotide of any one of embodiments 77-81, wherein the at least one modified internucleotide linkage of Formula (II) is located at the 3′ overhang of the double-stranded oligonucleotide.
Embodiment 85. The double-stranded oligonucleotide of any one of embodiments 77-84, wherein the at least one modified internucleotide linkage of Formula (II) is not located at the cut site of the passenger strand.
Embodiment 86. The double-stranded oligonucleotide of any one of embodiments 77-84, wherein the at least one modified internucleotide linkage of Formula (II) is located at the 5′ or 3′ end of the passenger strand.
Embodiment 87. The double-stranded oligonucleotide of any one of embodiments 77-84, wherein the at least one modified internucleotide linkage of Formula (II) is located at an internal position of the passenger strand.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. These examples are provided for illustrative purposes only and not to limit the scope of the claims provided herein.

### Example 1: Synthesis, Purification, and Analysis of Exemplary DAR2 Antibody-Oligonucleotide Conjugate (AOC) with siRNA Modified with PG Linkages

### Oligonucleotide Synthesis

MSTN: A 21-mer oligonucleotide duplex with 19 bases of complementarity against the mouse and human MSTN gene with a guide strand sequence of UUAUUAUUUGUUCUUUGCCUU (SEQ ID NO: 19) was generated. The guide and passenger strands were assembled on solid phase using standard phosphoramidite chemistry and purified by HPLC. The base, sugar, and phosphate modifications were used to optimize the potency of the duplex and reduce immunogenicity. The phosphoryl guanidine (PG) linkage was introduced at specific locations within the oligonucleotide by using 2-azido-1,3-dimethylimidazolium hexafluorophosphate in the oxidation step. Purified single strands were duplexed to get the double-stranded siRNA as described in Table 1.

TABLE 1
Exemplary Nucleic Acid Sequences of the Antisense Strands and the Sense
Strands

| | | SEQ | | SEQ |
|---|---|---|---|---|
| | Antisense strand sequence | ID | Sense strand sequence | ID |
| R# | (5′-3′) | NO: | (5′-3′) | NO: |
| R2336 | VpUmsUfsAUUAfUUUGUUCUfU | 1 | aminoC6- | 2 |
| | UfGCCsUsU | | GsGsCAAAGfAfAfCAAAUAAUsAsA | |
| R3552 | vpUqsUfsAUUAfUUUGUUCUfU | 3 | aminoC6- | 2 |
| | UfGCCsUpgU | | GsGsCAAAGfAfAfCAAAUAAUsAsA | |
| SN4-7 | vpUqsUfsAUUAfUUUGUUCUfU | 3 | aminoC6- | 9 |
| | UfGCCsUsU | | GpgGpgCpgAAAGfAfAfCAAAUAApg | |
| | | | UpgApgA | |
| SN4-8 | vpUqsUfsAUUAfUUUGUUCUfU | 3 | aminoC6- | 10 |
| | UfGCCsUsU | | GpgGpgCpgApgAAGfAfAfCAAAUAp | |
| | | | gApgUpgApgA | |
| SN4-9 | vpUqsUfsAUUAfUUUGUUCUfU | 3 | aminoC6- | 11 |
| | UfGCCsUsU | | GpgGpgCpgApgApgAGfAfAfCAAAU | |
| | | | pgApgApgUpgApgA | |
| SN4- | vpUqsUfsAUUAfUUUGUUCUfU | 3 | aminoC6- | 12 |
| 10 | UfGCCsUsU | | GpgGpgCpgApgApgApgGfAfAfCAAA | |
| | | | pgUpgApgApgUpgApgA | |
| SN4- | vpUqsUfsAUUAfUUUGUUCUfU | 3 | aminoC6- | 13 |
| 11 | UfGCCsUsU | | GpgGpgCpgApgApgApgGfpgAfAfCA | |
| | | | ApgApgUpgApgApgUpgApgA | |
| SN4- | vpUqsUfsAUUAfUUUGUUCUfU | 3 | aminoC6- | 14 |
| 12 | UfGCCsUsU | | GpgGpgCpgApgApgApgGfpgAfpgAfC | |
| | | | ApgApgApgUpgApgApgUpgApgA | |
| SN4- | vpUqsUfsAUUAfUUUGUUCUfU | 3 | aminoC6- | 15 |
| 13 | UfGCCsUsU | | GpgGpgCpgApgApgApgGfpgAfpgAfpg | |
| | | | CpgApgApgApgUpgApgApgUpgApgA | |
| R3557 | vpUqsUfsAUUAfUUUGUUCUfU | 3 | aminoC6- | 18 |
| | UfGCCsUpgU | | GpgGpgCAAAGfAfAfCAAAUAAUpg | |
| | | | ApgA | |
| R3553 | vpUqsUfAUUAfUUUGUUCUfUU | 4 | aminoC6- | 2 |
| | fGCCpgUsU | | GsGsCAAAGfAfAfCAAAUAAUsAsA | |
| R3558 | vpUqsUfsAUUAfUUUGUUCUfU | 4 | aminoC6- | 18 |
| | UfGCCpgUsU | | GpgGpgCAAAGfAfAfCAAAUAAUpg | |
| | | | ApgA | |
| | vpUqsUfsAUUAfUUUGUUCUfU | 5 | aminoC6- | 2 |
| | UfGCCpgUpgU | | GGCAAAGfAfAfCAAAUAAUAA | |
| | vpUqsUfpgAUUAfUUUGUUCUfU | 6 | aminoC6- | 2 |
| | UfGCCpgUpgU | | GGCAAAGfAfAfCAAAUAAUAA | |
| | UpgUfpgAUUAfUUUGUUCUfUU | 7 | aminoC6- | 2 |
| | fGCCpgUpgU | | GGCAAAGfAfAfCAAAUAAUAA | |
| R2493 | vpUqsUfsAUUAfUUUGUUCUfU | 8 | aminoC6- | 2 |
| | UfGCCsUsU | | GsGsCAAAGfAfAfCAAAUAAUsAsA | |
| R3554 | vpUqsUfsAUUAfUUUGUUCUfU | 8 | aminoC6- | 16 |
| | UfGCCUU | | GpgGpgCAAAGfAfAfCAAAUAAUsA | |
| | | | SA | |
| R3555 | vpUqsUfsAUUAfUUUGUUCUfU | 8 | aminoC6- | 17 |
| | UfGCCUU | | GsGsCAAAGfAfAfCAAAUAAUpgAp | |
| | | | gA | |
| R3556 | vpUqsUfsAUUAfUUUGUUCUfU | 8 | aminoC6- | 18 |
| | UfGCCUU | | GpgGpgCAAAGfAfAfCAAAUAAUpg | |
| | | | ApgA | |

*Upper case without f (N) refers to 2′-O-methyl modified nucleotide (e.g., A refers to 2′-O-methyl adenosine); upper case with f (Nf) refers to 2′-fluoro modified nucleotide (e.g., Af refers to 2′-fluoro adenosine); and pg refers to phosphoryl guanidine internucleotide linkage. vpUq or vpUm refers to a vinyl phosphonate modified nucleotide as represented below: 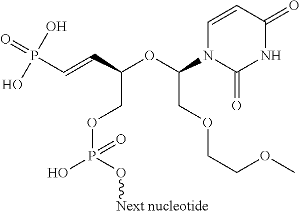
(vpUq) 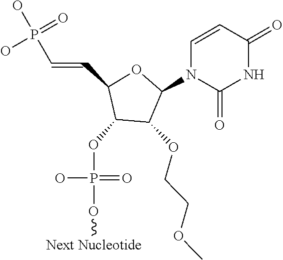
(vpUm).

### Anti-Transferrin Receptor Antibody

Anti-mouse transferrin receptor antibody or CD71 mAb is a rat IgG2a subclass monoclonal antibody that binds mouse CD71 or mouse transferrin receptor 1 (mTfR1). The antibody was produced by BioXcell, and it is commercially available (Catalog #BE0175).

### AOC Production

DAR2 AOCs were generated using a standard random cysteine conjugation method. The interchain disulfide bonds of the antibody were partially reduced with TCEP prior to conjugation with a maleimide linker-siRNA. The reaction mixture was purified using strong anion exchange chromatography method 2 to isolate a drug-antibody ratio (DAR) equal to 2 (i.e., two siRNAs per antibody). Collected AOC fractions were concentrated, buffer exchanged into PBS and sterile filtered using a 0.2 μm filter. The purity of AOCs was assessed using strong anion exchange chromatography method 1, size exclusion chromatography, and SDS-PAGE.

### Purification Methods

### Strong Anion Exchange (SAX) Method for Analyzing Conjugates

MPA: 10 mM Tris pH 7.2 20% Ethanol
MPB: 10 mM Tris, 1.5M NaCl pH 7.2 20% Ethanol
Column: Thermo Scientific, ProPac™ SAX-10, Bio LC™, 4×250 mm
Flow rate=0.75 mL/min

Table 2 shows gradient used for purification.

TABLE 2

| T(min) | % B |
|---|---|
| 0 | 10 |
| 3 | 10 |
| 11 | 60 |
| 14 | 60 |
| 15 | 80 |
| 15 | 80 |
| 16 | 10 |
| 20 | 10 |

### Non-Denaturing IP-RP Method

MPA: 1% HFIP, 0.2% TEA, 50 nM EDTA
MPB: Methanol
Column: Acquity premier oligonucleotide C18 Column 1.7 μm 2.1×50 mm
Flow rate: 0.6 mL/min
T=25° C.

Table 3 shows gradient used for purification.

TABLE 3

| T(min) | % B |
|---|---|
| 0 | 5 |
| 2 | 5 |
| 8 | 55 |
| 9 | 95 |
| 10 | 95 |
| 11 | 5 |
| 12 | 5 |

### Strong Anion Exchange Chromatography Method 2

Column: Tosoh Bioscience, TSKGel SuperQ-5PW, 21.5 mm ID×15 cm, 13 um
Solvent A: 20 mM TRIS buffer, pH 8.0; Solvent B: 20 mM TRIS, 1.5 M NaCl, pH 8.0
Flow Rate: 6.0 mL/min

Table 4 shows gradient used for purification.

TABLE 4

| % A | % B | Column volume |
|---|---|---|
| 100 | 0 | 1.00 |
| 60 | 40 | 18.00 |
| 40 | 60 | 2.00 |
| 40 | 60 | 5.00 |
| 0 | 100 | 2.00 |
| 100 | 0 | 2.00 |

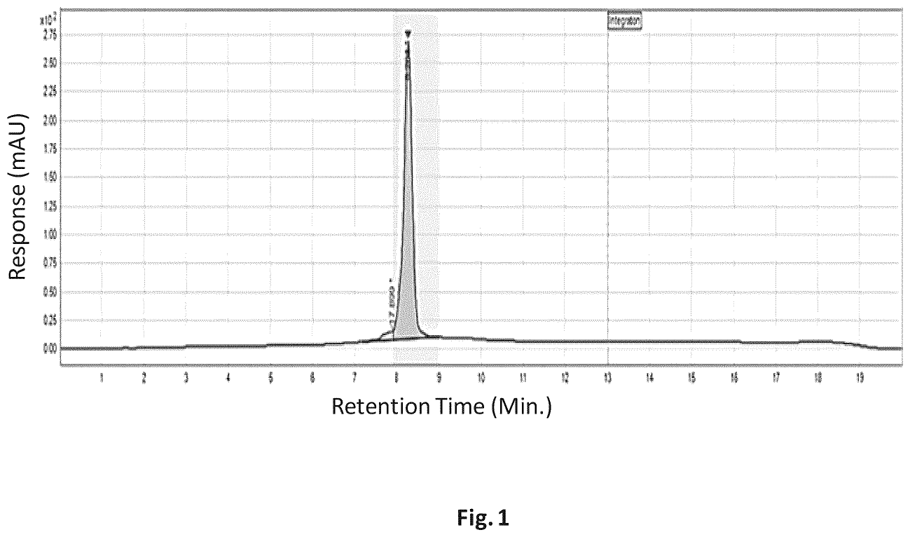

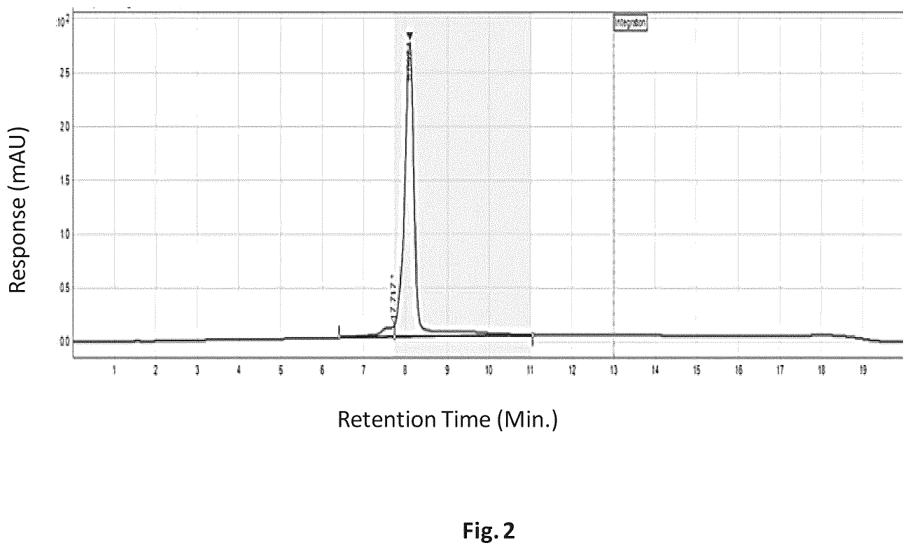

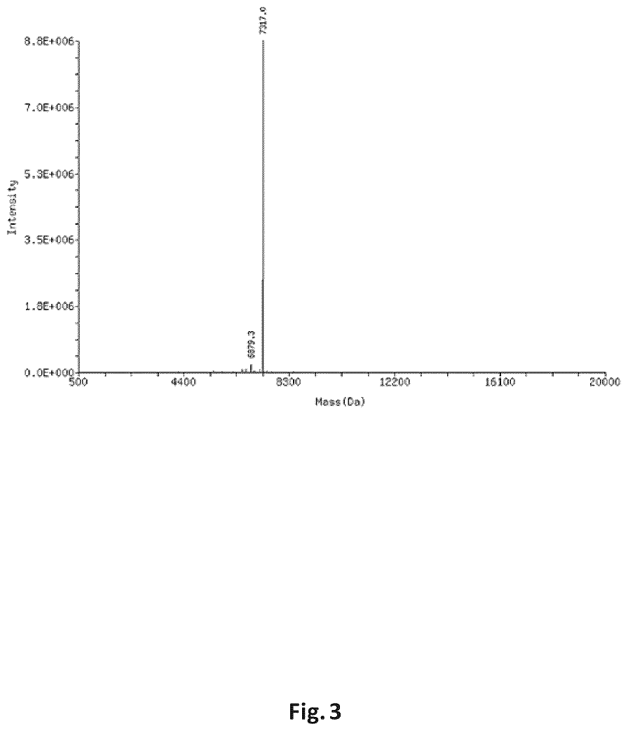

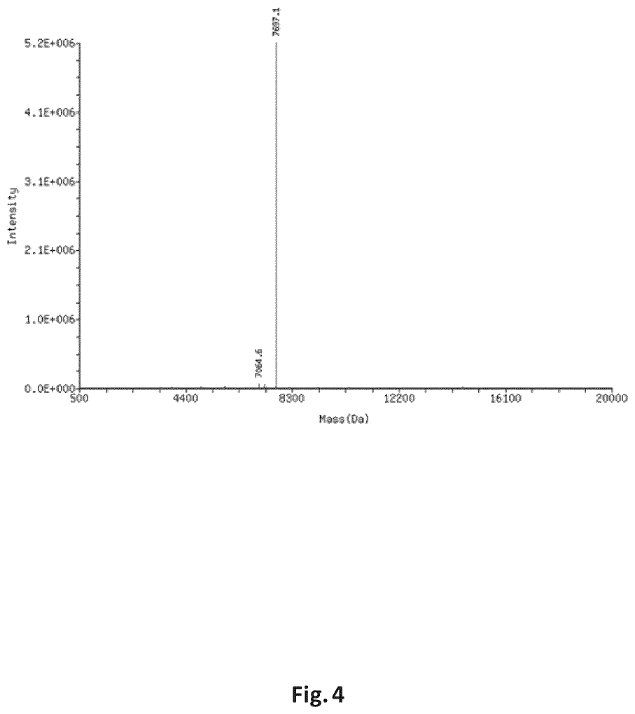

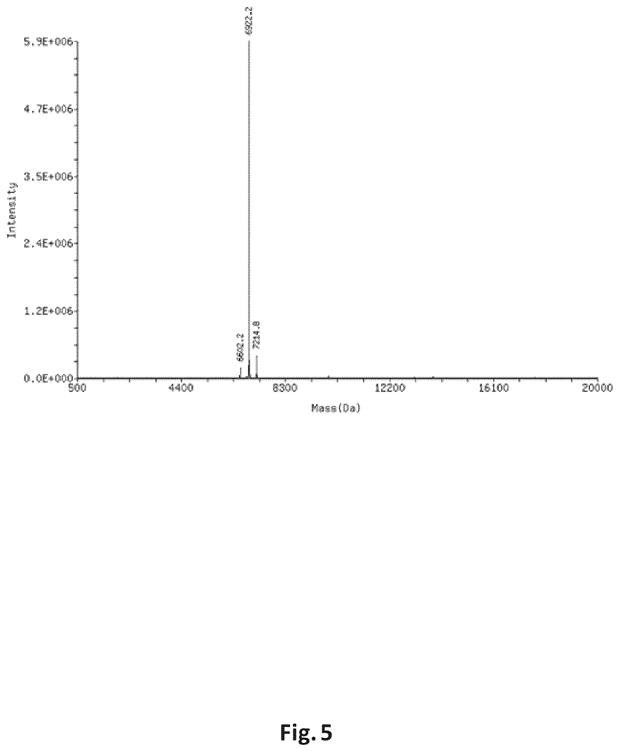

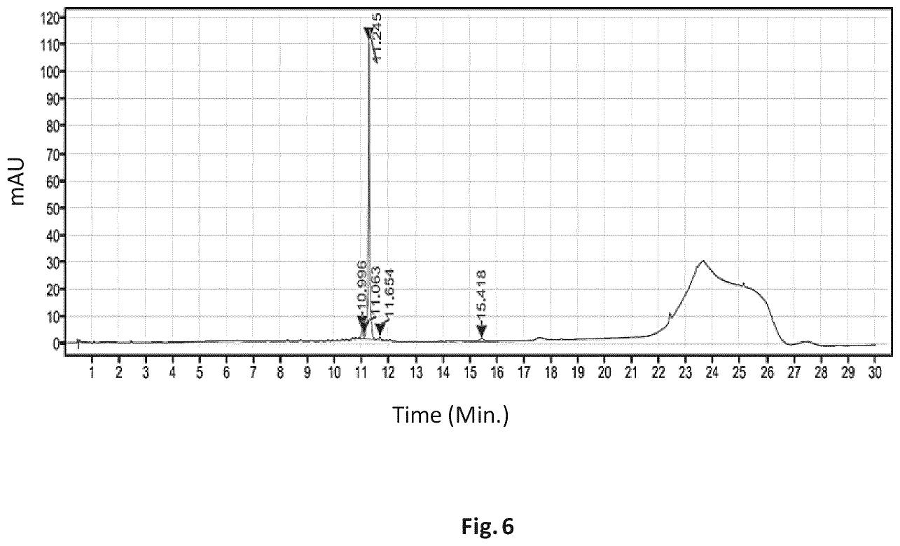

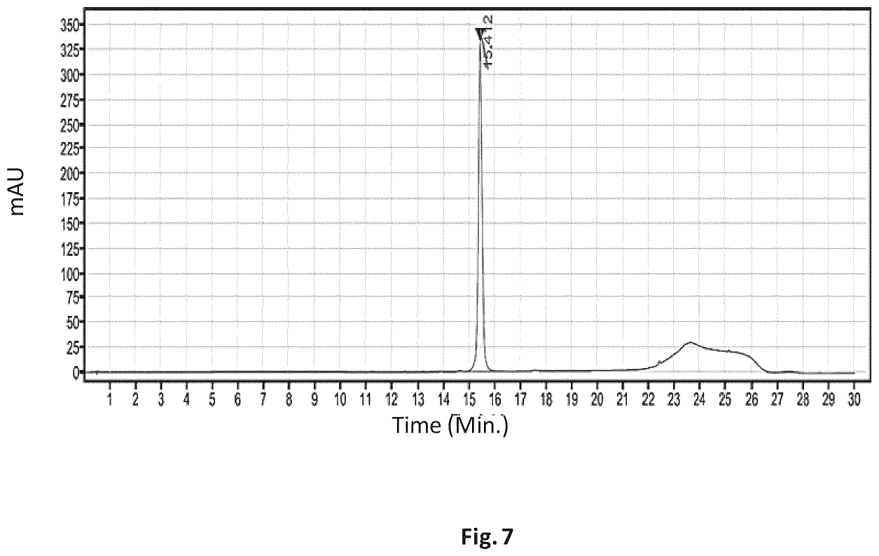

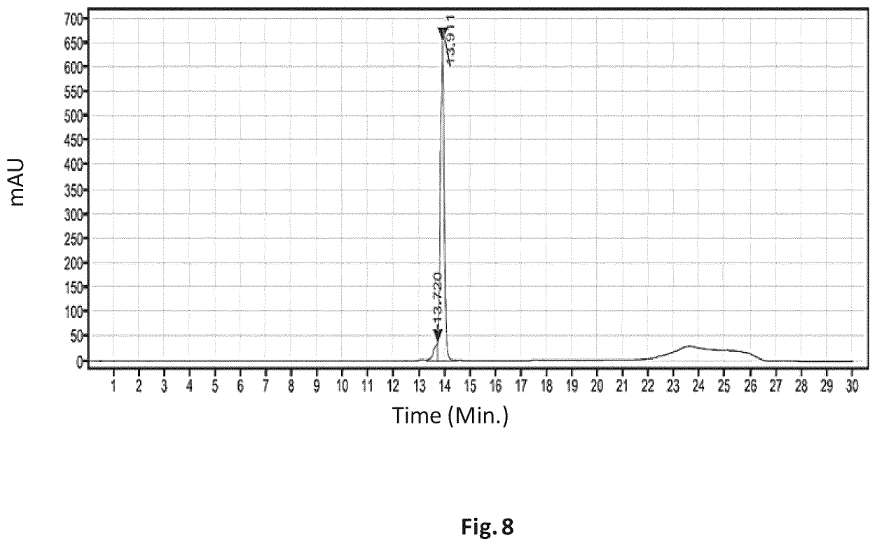

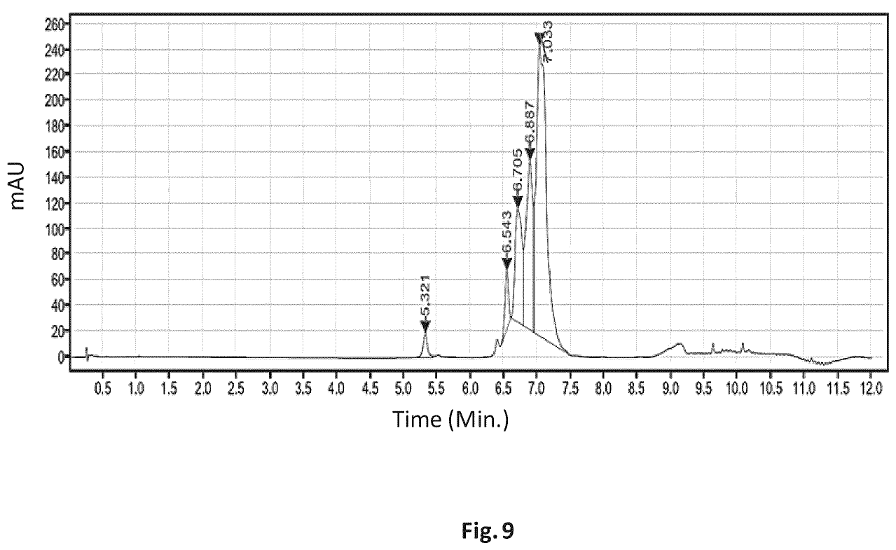

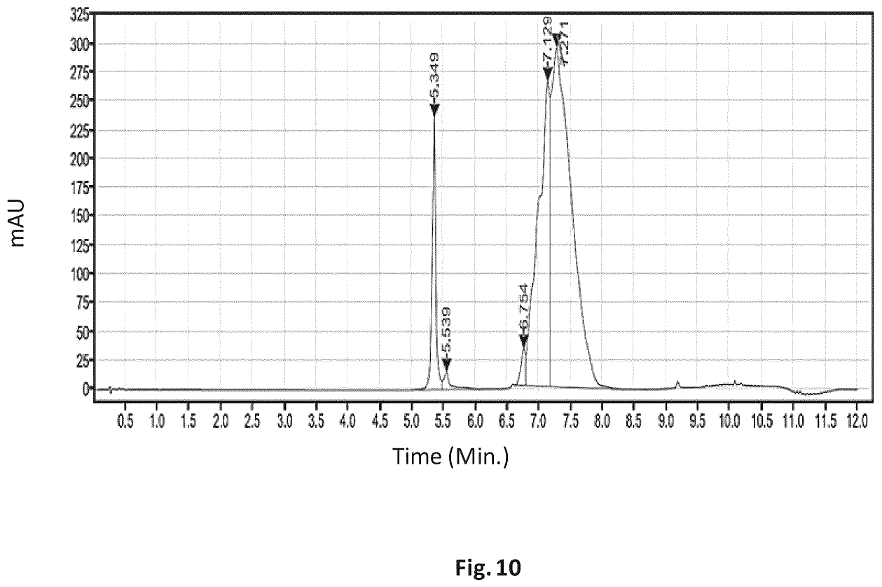

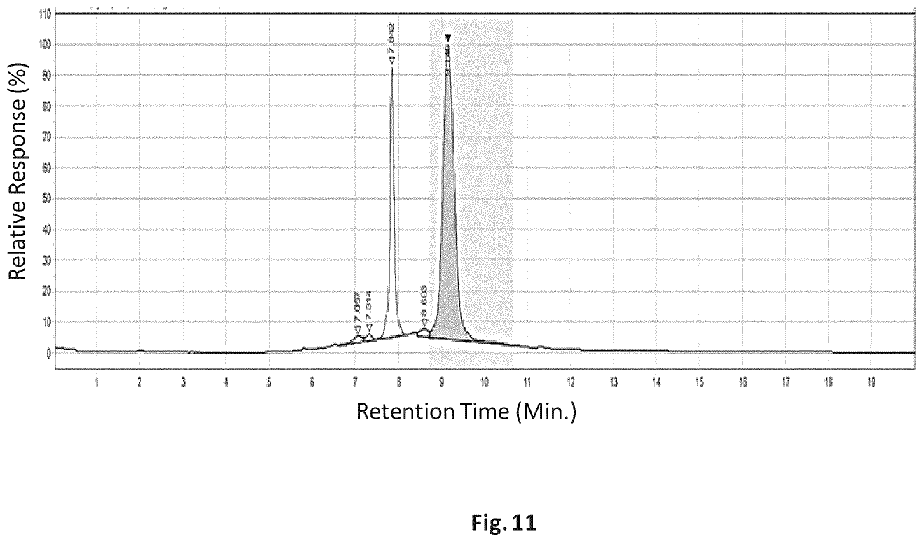

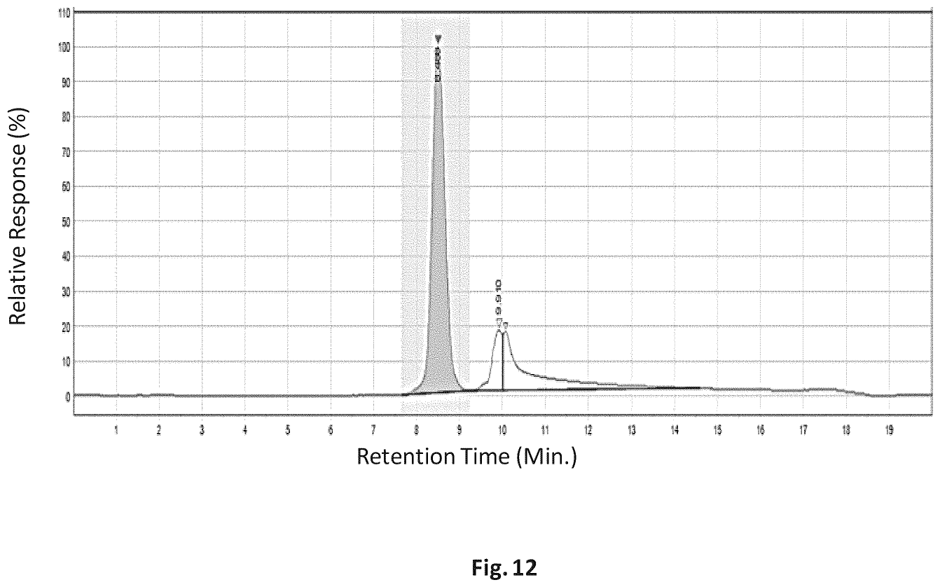

The analytical data for the DAR2 AOC with the siRNA modified with P=S and PG linkages are illustrated in FIGS. 1-12. Table 5 shows SAX retention times for DAR2 AOCs.

TABLE 5

| | | | | | AOC |
|---|---|---|---|---|---|
| | | SEQ ID | | SEQ ID | SAX RT |
| R# | GS | NO: | PS | NO: | DAR2 |
| R2336 | vpUmsUfsauuAfuuuguuc | 21 | aminoC6- | 23 | 9.129 |
| | UfuUfgccsusu | | gsgscaaaGfAfAfcaaauaausasa | | |
| R3662 | vpUqsUfsauuAfuuuguuc | 22 | aminoC6- | 24 | 8.415 |
| | UfuUfgccsusu | | (gPG)(gPG)caaaGfAfAfcaaauaa(uPG) | | |
| | | | (aPG)a | | |
| R3668 | vpUqsUfsauuAfuuuguuc | 22 | aminoC6- | 25 | 8.264 |
| | UfuUfgccsusu | | (gPG)(gPG)(cPG)(aPG)aaGfAfAfcaaau | | |
| | | | (aPG)(aPG)(uPG)(aPG)a | | |
| R3669 | vpUqsUfsauuAfuuuguuc | 22 | aminoC6-(gPG)(gPG)(cPG)(aPG) | 26 | 8.097 |
| | UfuUfgccsusu | | (aPG) | | |
| | | | (aPG)GfAfAfcaa(aPG)(uPG)(aPG) | | |
| | | | (aPG)(uPG)(aPG)a | | |
| R3671 | vpUqsUfsauuAfuuuguuc | 22 | aminoC6-(gPG)(gPG)(cPG)(aPG) | 27 | 7.981 |
| | UfuUfgccsusu | | (aPG) | | |
| | | | (aPG)(GfPG)AfAfca(aPG)(aPG)(uPG) | | |
| | | | (aPG)(aPG)(uPG)(aPG)a | | |

*Lower case without f (n) refers to 2′-O-methyl modified nucleotide (e.g., a refers to 2′-O-methyl adenosine); upper case with f(Nf) refers to 2′-fluoro modified nucleotide (e.g., Af refers to 2′-fluoro adenosine); and nPG refers to a 2′-O-methyl modified nucleotide comprising phosphoryl guanidine internucleotide linkage at the 3′ (e.g., (aPG) refers to 2′-O-methyl adenosine having phosphoryl guanidine internucleotide linkage at its 3′. vpUq or vpUm refers to a vinyl phosphonate modified nucleotide as represented above in Table 1.

### Example 2: In Vivo Activity of DAR2 Anti-Transferrin Receptor 1 mAb Conjugated to MSTN siRNA Modified with PG Internucleotide Linkages

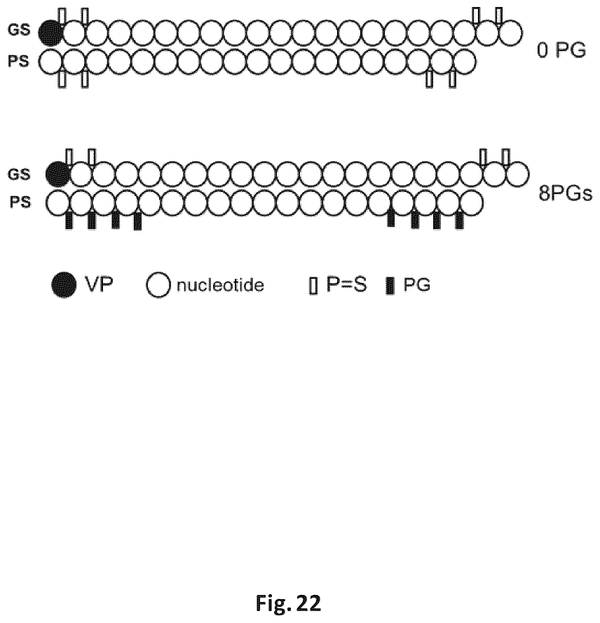

The DAR 2 AOCs were made and characterized as described in Example 1. The siRNAs of the DAR2 AOCs were modified by substituting phosphorothioate (P=S) linkages with phosphoryl guanidine (PG) linkages. FIG. 22 displays schemes of two siRNA molecules with phosphorothioate internucleotide linkages and/or phosphoryl guanidine (PG) linkages and the locations of such internucleotide linkages on the guide strand, passenger strand, or both strands of the siRNAs of the AOCs. The DAR2 AOC or conjugates were synthesized by conjugating two siRNAs to an anti-TfR1 antibody as described in Example 1.

The conjugates were assessed for their ability to mediate MSTN mRNA downregulation in muscle tissues in wild type CD-1 mice. Mice were dosed via intravenous (iv) injection with PBS vehicle control and the indicated DAR2 AOC at the constant antibody dose of 7.5 mg/kg. After 14, 28, or 56 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

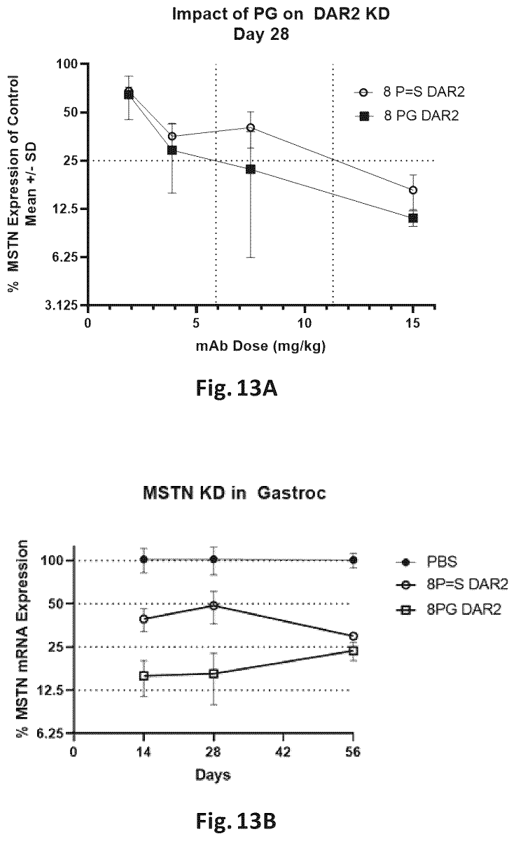

In vivo activities of increasing concentrations of DAR2 AOC with siRNAs modified with PG linkages on the passenger strands were evaluated for knock down activities of the myostatin mRNA levels in muscle cells from the gastrocnemius tissue in different doses (FIG. 13A). DAR2 AOC were generated with siRNA each comprising either 8 PG or 0 PG modification patterns (FIG. 22). 8 PG comprises siRNAs modified with 8 PG linkages, 4 PG linkages at the 5′ end and 4 PG linkages at the 3′ end on the passenger strand and 0 PG, or 8 P=S, comprises 2 phosphorothioate (P=S) internucleotide linkages at the 5′ end and 2 P=S internucleotide linkages at the 3′ end on the passenger strand. DAR2 AOC siRNA with the 8 PG pattern showed greater knocked down MSTN mRNA activities compared with the activities of the DAR2 AOC siRNA with the 8 P=S pattern at Day 28. These results demonstrate that the presence of PG linkages and absence of PS linkages on the passenger strand seen in 8 PG improved the DAR2 AOC activity compared to the DAR2 AOC activity of 8 P=S, where the siRNAs have PS linkages and no PG linkages.

In vivo activities of DAR2 AOC with siRNA modified with PG or P=S linkages on the passenger strands were evaluated for knock down of the myostatin mRNA levels in muscle cells from the gastrocnemius tissue in a time dependent manner (FIG. 13B). At Day 14 and Day 28, the siRNA modified with the 8 PG pattern showed MSTN mRNA downregulation of greater than 75%. The siRNA comprising a passenger strand without any PG linkages (8 P=S) was able to downregulate MSTN mRNA by around 50%. The presence of the 8 PG pattern of internucleotide linkages on the passenger strands of the siRNA improved the knock down activity of the DAR2 AOC compared to the DAR2 AOC activity of the 8 P=S pattern.

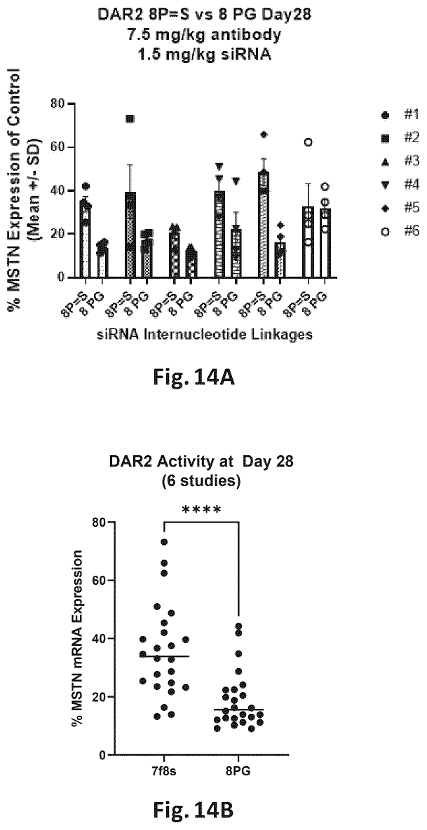

Additional experiments were conducted to evaluate the activity of the DAR2 AOC with siRNA wherein the passenger strands of the siRNA are modified with PG or P=S internucleotide linkages. All of the experiments showed that the DAR2 conjugates with siRNA having PG internucleotide linkages on the passenger strands induced greater downregulation of MSTN mRNA levels than the ones without any PG (8 P=S) internucleotide linkages (FIG. 14A). Analysis of the distribution of decreases in the mRNA levels indicates that the presence of PG internucleotide linkages on the passenger strands increases the mRNA knockdown activities of the DAR 2 conjugates (FIG. 14B).

Overall, these results indicate that DAR2 AOC with two siRNA modified with PG internucleotide linkages have better in vivo knock down activities than the ones for DAR2 AOC with two siRNA containing phosphorothioate internucleotide linkages.

### Example 3: In Vivo Activity of DAR2 Anti-Transferrin Receptor 1 mAb Conjugated to MSTN siRNA Modified with PG Internucleotide Linkages

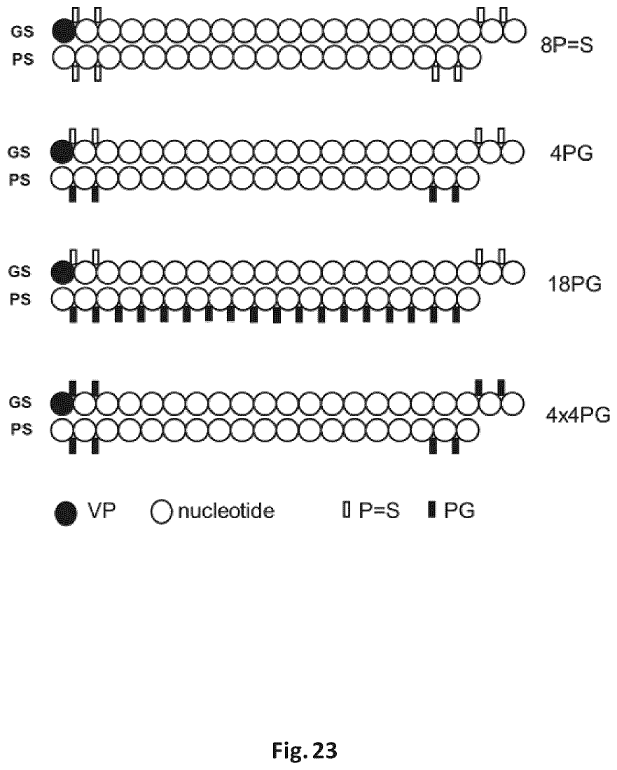

The DAR 2 AOCs were made and characterized as described in Example 1. The siRNAs of the DAR2 AOCs were modified by substituting the phosphorothioate (P=S) linkages with phosphoryl guanidine (PG) linkages. FIG. 23 illustrates schemes of four siRNA molecules with P=S linkages and/or PG linkages and the locations of the internucleotide linkages on the guide strand, passenger strand, or both strands of the siRNAs of the DAR2 AOCs. The DAR2 AOC or conjugates were synthesized by conjugating two siRNAs to an anti-TfR1 antibody as described in Example 1.

### In Vivo Study Design

Groups (n=4) of wild-type female CD-1 mice were treated with one intravenous (i.v.) tail vein injections of DAR2 siRNA antibody conjugates. Treatment groups received 10 mg/kg (based on the weight of mAb), and all groups were administered a dose volume of 5.0 mL/kg. Mice were sacrificed at 24, 72, or 168 hours post injection. Terminal blood samples were collected via cardiac puncture and processed to generate plasma for PK analysis.

Stem-Loop qPCR Assay for Quantification of siRNA

Plasma samples were directly diluted in TE buffer with 0.1% Triton-X. 50 mg tissue pieces were homogenized in 1 mL of Trizol using a TillueLyser II tissue homogenizer (Qiagen) and then diluted in TE buffer with 0.10% Triton-X. Standard curves were generated by spiking siRNA into plasma or homogenized tissue from untreated animals and then serially diluting with TE buffer with 0.1% Triton-X. The antisense (guide) strand of the siRNA was reverse transcribed using a TaqMan MicroRNA reverse transcription kit (Applied Biosystems) with 94 nM of a sequence-specific stem-loop RT primer. The cDNA from the RT step was utilized for real-time PCR using TaqMan Fast Advanced Master Mix (Applied Biosystems) with 1.5 μM of forward primer, 0.75 μM of reverse primer, and 0.2 μM of probe. Quantitative PCR reactions were performed using standard cycling conditions in a ViiA 7 Real-Time PCR System (Life Technologies). The Ct values were transformed into plasma or tissue concentrations using the linear equations derived from the standard curves.

### Results

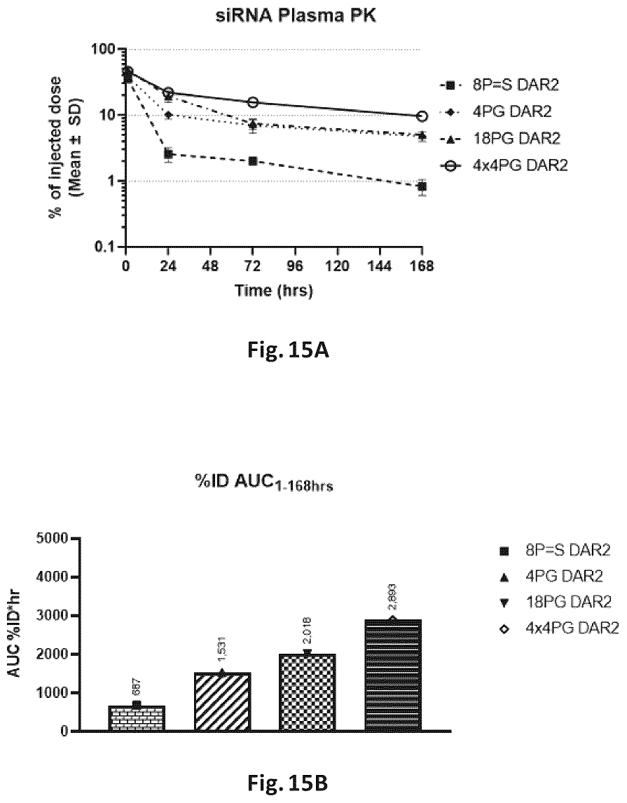

FIGS. 15A and 15B show the time dependent plasma pharmacokinetics of siRNA upon the single dose injection of DAR2 AOC with PG modified siRNA (10 mg/kg of mAb) administered to mice at 24, 72, and 168 hours post injection (FIG. 15A), and the calculated area under the curve (AUC) for the PG modified siRNA after 168 hours post injection (FIG. 15B). These results indicate that the DAR2 AOC with PG modified siRNAs (4 PG DAR2, 18 PG DAR2, and 4×4 PG DAR2; structures and locations of modified internucleotide linkages shown in FIG. 23 showed higher plasma concentration and higher AUC at least up to 168 hours after the injection than those of DAR2 AOC with siRNAs not modified any PG linkages (8 P=S).

FIG. 15A shows plasma levels of siRNA of DAR2 AOC with siRNA having unmodified guide strands and with the entire backbone of the passenger strands modified with PG (18 PG) were similar to the levels of the siRNA with unmodified guide strands and 4 PG linkages on the passenger strands (4 PG). Interestingly, the highest siRNA plasma levels were measured with DAR2 AOC having the siRNA modified with 4 PG internucleotide linkages on the guide strands and 4 PG internucleotide linkages on the passenger strands (4×4PG) for all time points.

FIG. 15B shows the AUC of these RNA with or without any PG internucleotide linkages of the DAR2 AOC were calculated at 168 hours post injection. The results indicate that the siRNA modified with the 4×4 PG pattern had the highest AUC. In addition, the siRNA without any PG internucleotide linkages of the guide strands or passenger strands (8 P=S) had the lowest AUC. The addition of PG linkages on the passenger strands with unmodified guide strands (4 PG and 18 PG) of the siRNA improved the AUC of the modified siRNA. The addition of PG linkages to the guide strand and passenger strand (4×4 PG) showed the greatest AUC comparatively.

Taken together, the PK data analysis indicates that the reduction of the clearance of the modified siRNA of the DAR2 AOC resulting in increased siRNA plasma levels correlates the number of PG linkages on the guide strands. The presence of 4 PG interlinkages at the 5′ and 3′ of the guide strands of the DAR2 AOC provides highest retention of siRNA in the plasma while the absence of PG internucleotide linkages results in much lower plasma retention. Therefore, these results indicate that reduction of the negative charge on the siRNA of the DAR2 AOC by having PG linkages instead of P=S linkages as demonstrated by higher levels of circulating siRNA in plasma.

### Example 4: In Vivo Activity of DAR2 Anti-Transferrin Receptor 1 mAb Conjugated to SSB or MSTN siRNA Modified with PG Internucleotide Linkages

### Oligonucleotide Synthesis

SSB: A 21-mer oligonucleotide duplex with 19 bases of complementarity and 3′ dinucleotide overhangs was designed against mouse small RNA binding exonuclease protection factor La (SSB). The sequence (5′ to 3′) of the guide/antisense strand was UUACAUUAAAGUCUGUUGUUU (SEQ ID NO: 20). MSTN: A 21-mer oligonucleotide duplex with 19 bases of complementarity against the mouse and human MSTN gene with a guide strand sequence of UUAUUAUUUGUUCUUUGCCUU (SEQ ID NO: 19) was generated. The guide and passenger strands were assembled on solid phase using standard phosphoramidite chemistry and purified by HPLC. The base, sugar, and phosphate modifications were used to optimize the potency of the duplex and reduce immunogenicity. The phosphoryl guanidine (PG) linkage was introduced within the oligonucleotide at specific locations by using 2-azido-1,3-dimethylimidazolium hexafluorophosphate in the oxidation step. Purified single strands were duplexed to get the double-stranded siRNA described above.

The siRNAs were modified on the passenger strands with up to 12 phosphoryl guanidine (PG) linkages for SSB siRNA or 15 PG linkages for MSTN siRNAd. The DAR2 AOC or conjugates were synthesized by conjugating two siRNAs to an anti-TfR1 antibody as described in Example 1.

The conjugates were assessed for their ability to mediate SSB mRNA downregulation in muscle tissues in wild type CD-1 mice. Mice were dosed via intravenous (iv) injection with PBS vehicle control and the indicated ASCs at 5 mg/kg. After 28 days, gastrocnemius (gastroc) and heart tissues were harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

Stem-Loop qPCR Assay for Quantification of siRNA

Plasma samples were directly diluted in TE buffer with 0.1% Triton-X. 50 mg tissue pieces were homogenized in 1 mL of Trizol using a TillueLyser II tissue homogenizer (Qiagen) and then diluted in TE buffer with 0.10% Triton-X. Standard curves were generated by spiking siRNA into plasma or homogenized tissue from untreated animals and then serially diluting with TE buffer with 0.10% Triton-X. The antisense strand of the siRNA was reverse transcribed using a TaqMan MicroRNA reverse transcription kit (Applied Biosystems) with 94 nM of a sequence-specific stem-loop RT primer. The cDNA from the RT step was utilized for real-time PCR using TaqMan Fast Advanced Master Mix (Applied Biosystems) with 1.5 μM of forward primer, 0.75 μM of reverse primer, and 0.2 μM of probe. Quantitative PCR reactions were performed using standard cycling conditions in a ViiA 7 Real-Time PCR System (Life Technologies). The Ct values were transformed into plasma or tissue concentrations using the linear equations derived from the standard curves.

### Results

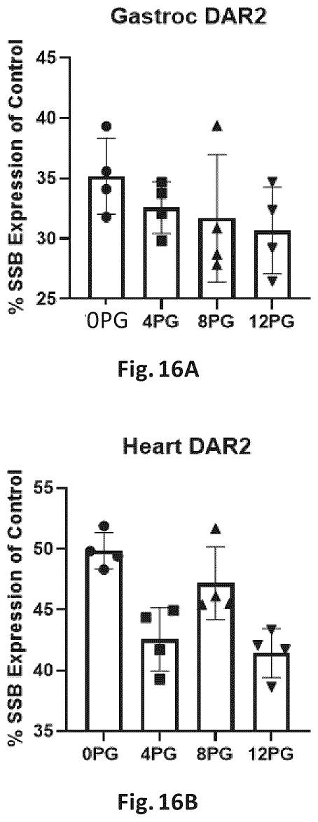

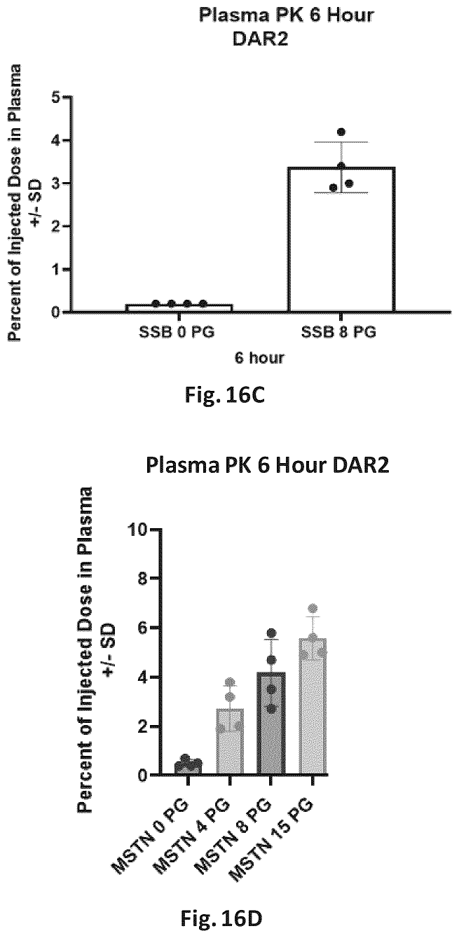

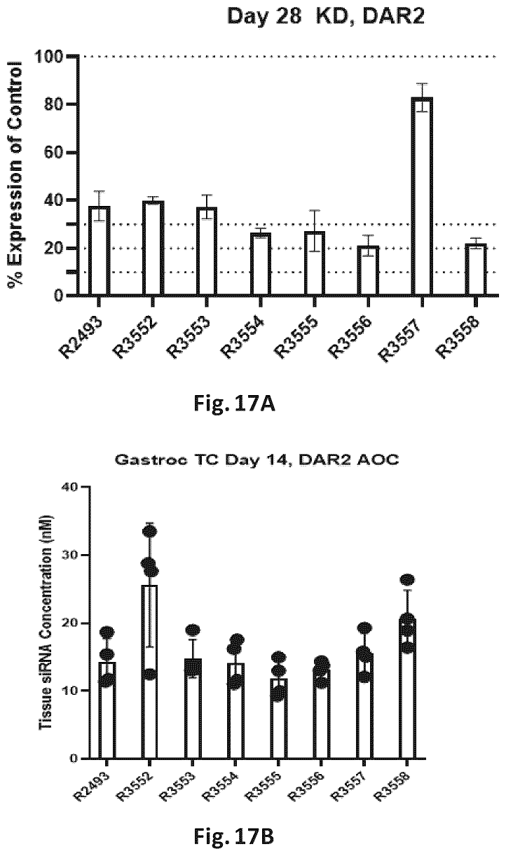

In vivo activities of increasing concentrations of DAR2 AOC with siRNA modified with 4, 8, and 12 PG linkages (the passenger strand has either 2 PG linkages at the 5′ end and at the 3′end, 4 PG linkages at the 5′ end and at the 3′end, or 6 consecutive PG linkages at the 5′end and 3′ end) on the passenger strands were evaluated for knock down activities of the SSB mRNA levels in muscle cells from the gastrocnemius tissue (FIG. 16A) and heart tissue (FIG. 16B). The DAR2 AOC with the siRNA modified with 4 PG, 8 PG, and 12 PG patterns showed greater knocked down SSB mRNA activities compared the activities of the DAR2 AOC with the unmodified siRNA at Day 28 (FIGS. 16A and 16B). The DAR2 AOC with the siRNA modified with 12 PG showed the most SSB mRNA knockdown in both tissues. In addition, the plasma studies indicate that levels of the SSB siRNA modified with 8 PG were much higher than levels of unmodified (0 PG) siRNA 6-hour post injection (FIG. 16C). Additionally, the plasma studies performed with 0 PG, 4 PG, 8 PG, and 15 PG MSTN DAR2 AOC showed that levels of the siRNA modified with increasing PG modifications had much higher than levels of unmodified (0 PG) siRNA 6-hour post injection (FIG. 16D).

These results indicate that the presence of at least 4 PG linkages on the passenger strand of the two siRNAs conjugated to the antibody improves the DAR2 AOC activity by increasing KD activities and increasing the stability of the AOC molecules as suggested by the elevated siRNA plasma concentrations.

Taken together, biological activities of the DAR2 AOC were enhanced by having the passenger strand modified with at least 4 PG linkages. These results also demonstrate that enhancements were not gene specific, since they apply to at least two different genes tested: MSTN and SSB.

### Example 5: In Vivo Activity of DAR2 Anti-Transferrin Receptor 1 mAb Conjugated to MSTN siRNA Modified with PG Internucleotide Linkages

siRNAs were made and characterized as described in Example 1. The siRNAs were modified by substituting the phosphorothioate linkages (P=S) on the guide strand or the passenger strand with phosphoryl guanidine (PG) linkages. Table 6 displays the locations of the internucleotide linkages that were modified with phosphoryl guanidine (PG) linkages on the guide strand, the passenger strand, or both strands of the siRNAs. The DAR2 AOC or conjugates were synthesized by conjugating two siRNAs to an anti-TfR1 antibody as described in Example 1.

TABLE 6

| | | SEQ | | SEQ ID |
|---|---|---|---|---|
| R# | GS | ID NO: | PS | NO: |
| R2336 | vpUmsUfsauuAfuuuguucUfuUfgccsusu | 21 | aminoC6-gsgscaaaGfAfAfcaaauaausasa | 23 |
| R2493 | vpUqsUfsauuAfuuuguucUfuUfgccsusu | 22 | aminoC6-gsgscaaaGfAfAfcaaauaausasa | 23 |
| R3552 | vpUqsUfsauuAfuuuguucUfuUfgccs(uPG)u | 28 | aminoC6-gsgscaaaGfAfAfcaaauaausasa | 23 |
| R3553 | vpUqsUfsauuAfuuuguucUfuUfgc(cPG)usu | 29 | aminoC6-gsgscaaaGfAfAfcaaauaausasa | 23 |
| R3554 | vpUqsUfsauuAfuuuguucUfuUfgccsusu | 22 | aminoC6- | 30 |
| | | | (gPG)(gPG)caaaGfAfAfcaaauaausasa | |
| R3555 | vpUqsUfsauuAfuuuguucUfuUfgccsusu | 22 | aminoC6- | 31 |
| | | | gsgscaaaGfAfAfcaaauaa(uPG)(aPG)a | |
| R3556 | vpUqsUfsauuAfuuuguucUfuUfgccsusu | 22 | aminoC6- | 24 |
| | | | (gPG)(gPG)caaaGfAfAfcaaauaa(uPG) | |
| | | | (aPG)a | |
| R3557 | vpUqsUfsauuAfuuuguucUfuUfgccs(uPG)u | 28 | aminoC6- | 24 |
| | | | (gPG)(gPG)caaaGfAfAfcaaauaa(uPG) | |
| | | | (aPG)a | |
| R3558 | vpUqsUfsauuAfuuuguucUfuUfgc(cPG)usu | 29 | aminoC6- | 24 |
| | | | (gPG)(gPG)caaaGfAfAfcaaauaa(uPG) | |
| | | | (aPG)a | |

*Lower case without f (n) refers to 2′-O-methyl modified nucleotide (e.g., a refers to 2′-O-methyl adenosine); upper case with f (Nf) refers to 2′-fluoro modified nucleotide (e.g., Af refers to 2′-fluoro adenosine); and (nPG) refers to a 2'-O-methyl modified nucleotide comprising phosphoryl guanidine internucleotide linkage at the 3' (e.g., (aPG) refers to 2′-O-methyl adenosine having phosphoryl guanidine internucleotide linkage at its 3′. vpUq or vpUm refers to a vinyl phosphonate modified nucleotide as represented above in Table 1.

The conjugates were assessed for their ability to mediate MSTN mRNA downregulation in muscle tissues in wild type CD-1 mice. Mice were dosed via intravenous (iv) injection with PBS vehicle control and the indicated DAR2 AOC at the constant antibody dose of 10 mg/kg and siRNA dose of 2 mg/kg. After 28 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

In vivo activities of increasing concentrations of DAR2 AOC with siRNA modified with PG linkages substituting for the P=S linkages on the passenger and guide strands were evaluated for knock down activities of the MSTN mRNA levels in muscle cells from the gastrocnemius tissue (FIG. 17A) and siRNA tissue concentrations (FIG. 17B). FIG. 17A shows a bar graph quantifying the mRNA % expression of MSTN relative to control for the DAR2 AOC treatments. The two siRNAs (R3552 and R3553) with the passenger strand having no PG internucleotide linkage and the guide strand modified with a PG linkage substituting for the P=S linkage on the 3′ end did not any effect for the knock down activity of the DAR2 AOC. However, the two siRNAs (R3554 and R3555) with the guide strands having no PG internucleotide linkage and the passenger strands modified with the 2 PG linkages substituting for the 2 P=S linkages on the 5′ end or 3′ end improved the knock down activity of the DAR2 AOC. The siRNA (R3556) with the guide strand having no PG internucleotide linkage and the passenger strand modified with the 2 PG linkages substituting for the 2 P=S linkages on the 5′ end and 3′ end had the greatest knock down activity of the DAR2 AOC. The siRNA (R3557) of the DAR2 AOC with the guide strand modified with a PG linkage substituting for a P=S linkage on the 3′ overhang and the passenger strand modified with 2 PG linkages substituting for the 2 P=S linkages on the 5′ end and 3′ end showed low knock down activity among the siRNA of the DAR2 AOC. Interestingly, the siRNA (R3558) of the DAR2 AOC with the guide strand modified with a PG linkage substituting for a P=S linkage on the 3′ end and the passenger strand modified with 2 PG linkages substituting for the 2 P=S linkages on the 5′ end and 3′ end showed the greatest knock down activity of the DAR2 AOC about equal to that of R3556. This is interesting since the difference between R3557 and R3558 is the location of the PG linkage on the guide strand. This demonstrates the importance of the location of the siRNA modification.

FIG. 17B shows a bar graph quantifying the gastrocnemius tissue concentration of the siRNA treatments. The DAR2 AOC with the siRNA (3552) having a PG modified guide strand had the highest siRNA tissue concentration. All of the other DAR2 AOC with PG modified siRNA (R2492, R3553, R3554, R3555, R3556, R3557, and R3558) had relatively similar siRNA tissue concentrations, with R3558 being the greatest of that group. Overall, the presence of PG linkages on the siRNA of DAR2 AOC had insubstantial effects on the delivery of siRNA as measured by siRNA tissue concentrations.

Taken together, all siRNAs of DAR2 AOC were delivered in the tissue, and the siRNAs of DAR2 AOC with the guide strands substituting the P=S linkage with a PG linkage at the 3′ overhang and all 4 P=S substituted with PG on the passenger strand on the DAR2 AOC showed diminished knock down activity. These results suggest that DAR2 AOC with siRNA having the P=S linkage at the 3′ overhang cannot be substituted with a PG linkage on the guide strand.

### Example 6: In Vivo Activity of DAR2 Anti-Transferrin Receptor 1 mAb Conjugated to MSTN siRNA Modified with PG Internucleotide Linkages

siRNAs were made and characterized as described in Example 1. The siRNAs were modified on the passenger strand with 4, 8, 12, or 14 phosphoryl guanidine (PG) linkages. Table 7 displays the locations of the internucleotide linkages that were modified with phosphoryl guanidine (PG) linkages on the passenger strand of the siRNAs. The DAR2 AOC or conjugates were synthesized by conjugating two siRNAs to an anti-TfR1 antibody as described in Example 1.

TABLE 7

| | | SEQ | PS | SEQ ID | # |
|---|---|---|---|---|---|
| R# | GS | ID NO: | | NO: | PG |
| R2336 | vpUmsUfsauuAfuuuguucUfuUfgccsusu | 21 | aminoC6- | 23 | 0 |
| | | | gsgscaaaGfAfAfcaaauaausasa | | |
| R3662 | vpUqsUfsauuAfuuuguucUfuUfgccsusu | 22 | aminoC6- | 24 | 4 |
| | | | (gPG)(gPG)caaaGfAfAfcaaauaa | | |
| | | | (uPG)(aPG)a | | |
| R3668 | vpUqsUfsauuAfuuuguucUfuUfgccsusu | 22 | aminoC6- | 32 | 8 |
| | | | (gPG)(gPG)(cPG)(aPG)aaGfAfAf | | |
| | | | caaau(aPG)(aPG)(uPG)(aPG)a | | |
| R3669 | vpUqsUfsauuAfuuuguucUfuUfgccsusu | 22 | aminoC6- | 33 | 12 |
| | | | (gPG)(gPG)(cPG)(aPG)(aPG)(aP | | |
| | | | G)GfAfAfcaa(aPG) | | |
| | | | (uPG)(aPG)(aPG)(uPG)(aPG)a | | |
| R3671 | vpUqsUfsauuAfuuuguucUfuUfgccsusu | 22 | aminoC6- | 34 | 14 |
| | | | (gPG)(gPG)(cPG)(aPG)(aPG)(aP | | |
| | | | G)(GfPG)AfAfca(aPG)(aPG)(uP | | |
| | | | G)(aPG)(aPG)(uPG)(aPG)a | | |

*Lower case without f (n) refers to 2′-O-methyl modified nucleotide (e.g., a refers to 2′-O-methyl adenosine); upper case with f (Nf) refers to 2′-fluoro modified nucleotide (e.g., Af refers to 2′-fluoro adenosine); and (nPG) refers to a 2′-O-methyl modified nucleotide comprising phosphoryl guanidine internucleotide linkage at the 3′ (e.g., (aPG) refers to 2′-O-methyl adenosine having phosphoryl guanidine internucleotide linkage at its 3′. vpUq or vpUm refers to a vinyl phosphonate modified nucleotide as represented above in Table 1.

The conjugates were assessed for their ability to mediate MSTN mRNA

downregulation in muscle tissues in wild type CD-1 mice. Mice were dosed via intravenous (iv) injection with PBS vehicle control and the indicated DAR2 AOC at the constant antibody dose of 7 mg/kg and siRNA dose of 1.4 mg/kg. After 14 or 28 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

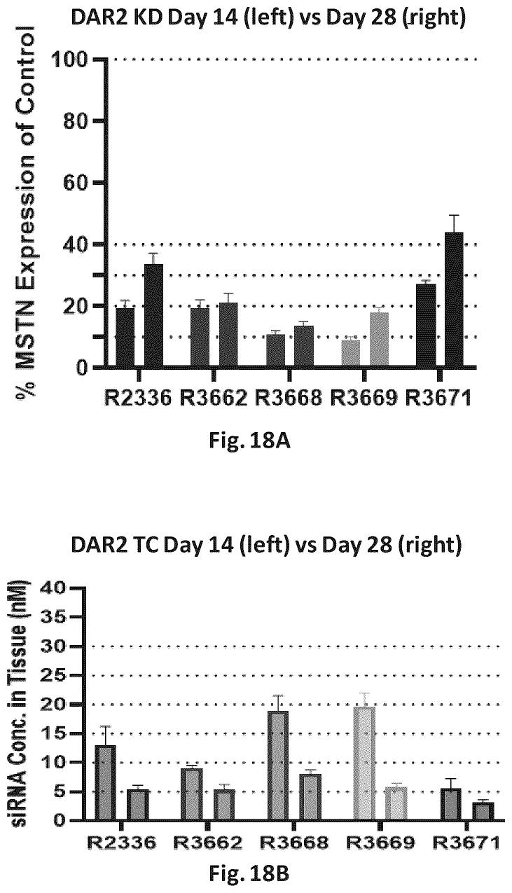

In vivo activities of DAR2 AOC with siRNA modified with 4, 8, 12, or 14 PG linkages on the passenger strands and unmodified guide strands (e.g., without a PG internucleotide linkage) were evaluated for knock down activities of the MSTN mRNA levels at day 14 (left) or day 28 (right) in muscle cells from the gastrocnemius tissue (FIG. 18A) and siRNA tissue concentrations (FIG. 18B). For all the siRNA modified with PG linkages on the passenger strands, the substitution of the 4 P=S linkages on the passenger strand with PG linkages did not affect the knock down activities of the siRNA of the DAR2 AOC.

FIG. 18A shows a bar graph of the % MSTN mRNA expressed relative to control. The increasing number of PG linkages on the passenger strand of the siRNA of the DAR2 AOC increased the knock down activities of siRNA at day 14 or day 28 of the siRNA when compared to the unmodified siRNA. The passenger strand of the siRNA modified with 8 PG (R3668) or 12 PG (R3669) linkages had the highest maximum knock down activities compared to 0 PG (R2336), 4 PG (R3662), and 14 PG (R3671)siRNAs

FIG. 18B shows a bar graph quantifying the gastrocnemius tissue concentration of the siRNA treatments. The results of the tissue concentration mirrored the % MSTN mRNA knock down of FIG. 18A. For example, the concentration of 8 PG and 12 PG was the highest out of the treatments at Days 14 and 28, while the 14 PG siRNA had low tissue concentrations of the siRNA which correlated to the low % MSTN mRNA knock down. Also, at Day 14, levels of siRNA were much higher than levels at Day 28.

The results indicate that DAR2 AOC having siRNA modified with 8 or 12 PG linkages provide the optimal KD activities and tissue concentrations and the absence of the P=S linkage on the passenger strand did not have any effect the activities or tissue concentration of the modified siRNA.

### Example 7: In Vivo Activity of DAR2 Anti-Transferrin Receptor 1 mAb Conjugated to MSTN siRNA Modified with 8 PG Internucleotide Linkages

siRNAs were made and characterized as described in Example 1. The siRNAs were modified on the guide strand or the passenger strand with 8 phosphoryl guanidine (PG) linkages. Table 8 displays the locations of the eight PG linkages on the passenger strand of the siRNAs. The DAR2 AOC or conjugates were synthesized by conjugating two siRNAs to an anti-TfR1 antibody as described in Example 1.

TABLE 8

| | | SEQ ID | |
|---|---|---|---|
| R# | Passenger strand (PS) | NO: | |
| 3837 | aminoC6- | 23 | OPG |
| | gsgscaaaGfAfAfcaaauaausasa | | |
| 4843 | aminoC6- | 35 | 8PG alt1 |
| | g[PG]g[PG]ca[PG]aa[PG]GfAfAfcaaa[PG]ua[PG]u[PG]a[PG]a | | |
| 4846 | aminoC6- | 36 | 8PG alt-2 |
| | g[PG]g[PG]caa[PG]a[PG]GfAfAfcaaa[PG]u[PG]aau[PG]a[PG]a | | |
| 4845 | aminoC6- | 37 | 8PG alt 3′ |
| | g[PG]g[PG]caaaGfAfAfcaaa[PG]u[PG]a[PG]a[PG]u[PG]a[PG]a | | |
| 4844 | aminoC6- | 38 | 8PG alt 5′ |
| | g[PG][PG]c[PG]a[PG]a[PG]a[PG]GfAfAfcaaauaau[PG]a[PG]a | | |
| 4892 | aminoC6- | 39 | 8PG + 1 |
| | [PG]g[PG]g[PG]c[PG]a[PG]aaGfAfAfcaaaua[PG]a[PG]u[PG]a[PG]a | | |

*Lower case without f (n) refers to 2′-O-methyl modified nucleotide (e.g., a refers to 2′-O-methyl adenosine); upper case with f (Nf) refers to 2′-fluoro modified nucleotide (e.g., Af refers 2′-fluoro adenosine); and [PG] refers to a phosphoryl guanidine internucleotide linkage.

The conjugates were assessed for their ability to mediate MSTN mRNA downregulation in muscle tissues in wild type CD-1 mice. Mice were dosed via intravenous (iv) injection with PBS vehicle control and the indicated DAR2 AOC at the constant antibody dose of 7.5 mg/kg and siRNA dose of 1.4 mg/kg. After 28 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

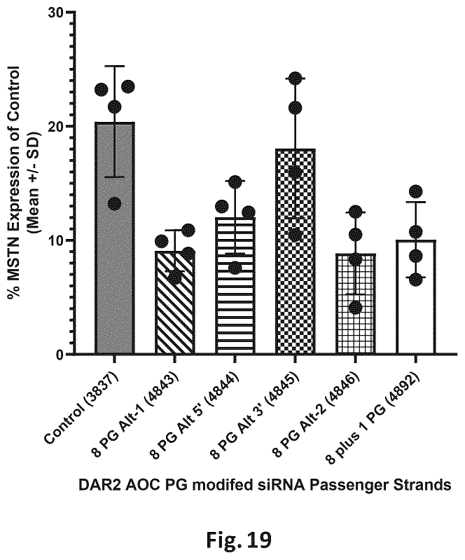

In vivo activities of DAR2 AOC with siRNA modified with 8 PG linkages on the passenger strand and unmodified guide strands were evaluated for knock down activities of the MSTN mRNA levels at day 28 in muscle cells from the gastrocnemius tissue (FIG. 19). The locations of the 8 PG linkages on the passenger strand varied between the siRNAs. The 8 PG linkages were either equally or unequally located on either side of the cut site of the passenger strand. As shown in FIG. 19, the two siRNAs with 4 PG linkages on either side the cut site (R4843 and R4846) had increased knockdown activity compared to the siRNA with 6 PG linkages on the 3′ end and 2 PG linkages of the 5′ end of the cut site (R4845). However, the presence of 2 PG linkages on the 3′ end and 6 PG linkages of the 5′ end of the cut site was able to somewhat restore the activity of the siRNA (R4844). The presence of one additional PG linkage adjacent the linker on the 5′ end of the passenger strand did not have a significant effect on the activity of the siRNA. Interestingly, the activity of the modified siRNA R4845 was similar to the unmodified siRNA.

Taken together, these results indicate that activities of the modified siRNA are dependent on the distribution and positioning of the PG linkages with respect to the cut site on the passenger strands. The best siRNA of the group was the siRNA modified with at least 4 PG linkages on the 3′ and 5′ end of the cut site. The siRNA modified with 6 PG linkages on the 3′ end of the cut site on the passenger strand had significant decrease in knockdown activity while the siRNA modified with 6 PG linkages on the 5′ end of the cut side of the passenger strand had increased activity comparatively.

### Example 8: In Vivo Activity of DAR2 Anti-Transferrin Receptor 1 mAb Conjugated to MSTN siRNA Modified with Up to 15 PG Internucleotide Linkages

siRNAs were made and characterized as described in Example 1. The siRNAs were modified on the guide strand or the passenger strand with up to 15 phosphoryl guanidine (PG) linkages. Table 9 displays the locations of the phosphoryl guanidine (PG) linkages on the guide strand of the siRNAs. The DAR2 AOC or conjugates were synthesized by conjugating two siRNAs to an anti-TfR1 antibody as described in Example 1.

TABLE 9

| | | SEQ ID | |
|---|---|---|---|
| R# | Passenger strand (PS) | NO: | |
| 3837 | aminoC6-gsgscaaaGfAfAfcaaauaausasa | 23 | 0PG |
| 4890 | aminoC6-[PG]gsgscaaaGfAfAfcaaauaausasa | 40 | 1PG at P0 |
| 4842 | aminoC6- | 41 | 12PG |
| | g[PG]g[PG]c[PG]a[PG]a[PG]a[PG]GfAfAfcaaa[PG]u[PG]a[PG]a | | |
| | [PG]u[PG]a[PG]a | | |
| 4884 | aminoC6- | 42 | 13PG |
| | g[PG]g[PG]c[PG]a[PG]a[PG]aGfAfAfca[PG]a[PG]a[PG]u[PG]a | | |
| | [PG]a[PG]u[PG]a[PG]a | | |
| 4894 | aminoC6- | 43 | 13PG +1 |
| | [PG]g[PG]g[PG]c[PG]a[PG]a[PG]aGfAfAfca[PG]a[PG]a[PG]u | | |
| | [PG]a[PG]a[PG]u[PG]a[PG]a | | |
| 4886 | aminoC6- | 44 | 14PG 3′ |
| | g[PG]g[PG]c[PG]a[PG]a[PG]aGfAfAfc[PG]a[PG]a[PG]a[PG]u | | |
| | [PG]a[PG]a[PG]u[PG]a[PG]a | | |
| 4888 | aminoC6- | 45 | 14PG 5′ |
| | g[PG]g[PG]c[PG]a[PG]a[PG]a[PG]GfAfAfca[PG]a[PG]a[PG]u | | |
| | [PG]a[PG]a[PG]u[PG]a[PG]a | | |
| 4847 | aminoC6-g[PG]g[PG]c[PG]a[PG]a[PG]a[PG]GfAfAfc[PG]a[PG] | 46 | 15PG |
| | a[PG]a[PG]u[PG]a[PG]a[PG]u[PG]a[PG]a | | |
| 4896 | aminoC6- | 47 | 15PG +1 |
| | [PG]g[PG]g[PG]c[PG]a[PG]a[PG]a[PG]GfAfAfc[PG]a[PG]a[PG] | | |
| | a[PG]u[PG]a[PG]a[PG]u[PG]a[PG]a | | |

*Lower case without f (n) refers to 2′-O-methyl modified nucleotide (e.g., a refers 2′-O-methyl adenosine); upper case with f (Nf) refers to 2′-fluoro modified nucleotide (e.g., Af refers 2′-fluoro adenosine); and [PG] refers to a phosphoryl guanidine internucleotide linkage.

The conjugates were assessed for their ability to mediate MSTN mRNA downregulation in muscle tissues in wild type CD-1 mice. Mice were dosed via intravenous (iv) injection with PBS vehicle control and the indicated DAR2 AOC at the constant antibody dose of 7.5 mg/kg and siRNA dose of 1.4 mg/kg. After 28 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

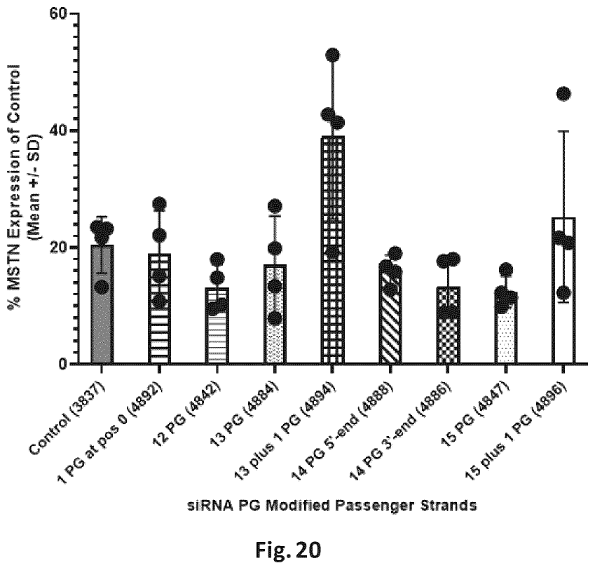

In vivo activities of DAR2 AOC with the siRNAs modified with 1 to 15 PG linkages on the passenger strands and unmodified guide strands were evaluated for knock down activities of the MSTN mRNA levels at day 28 in muscle cells from the gastrocnemius tissue (FIG. 20). The DAR2 AOC with siRNAs with PG modified passenger strand had greater knockdown activities than unmodified passenger strand. The passenger strands of the siRNA modified with up to 15 PG linkages had greater knock down activities when compared to control siRNA (R4847). The DAR2 AOC with the siRNAs with the most knockdown activity had passenger strands modified with 14 (R4886 and R4888) or 15 PG linkages (R4847). Interestingly, the presence of a sole PG linkage at the 5′ end adjacent to the linker in combination with other PG linkages on the passenger strand (R4894 or R4896) decreased the knockdown activity the siRNA not modified with a PG at that position.

These results indicate that DAR2 AOC activities can be increased with the presence of up to 15 PG linkages on the passenger strands. Taken together, the location of the PG linkages on the passenger strands affects the biological activity of the siRNAs conjugated to the antibody.

### Example 9 In Vivo Activity of DAR2 Anti-Transferrin Receptor 1 mAb Conjugated to MSTN siRNA Modified with PG Internucleotide Linkages

siRNAs were made and characterized as described in Example 1. The siRNAs were modified on the passenger strand with 4, 6, 8, and 12 phosphoryl guanidine (PG) linkages (the passenger strand has either 2 PG linkages at the 5′ end and at the 3′end, 3 PG linkages at the 5′ end and at the 3′end, 4 PG linkages at the 5′ end and at the 3′end, or 6 PG linkages at the 5′ end and at the 3′end). The DAR2 AOC or conjugates were synthesized by conjugating two siRNAs to an anti-TfR1 antibody as described in Example 1.

The conjugates were assessed for their ability to mediate MSTN mRNA downregulation in muscle tissues in wild type CD-1 mice. Mice were dosed via intravenous (iv) injection with PBS vehicle control and the indicated DAR2 AOC at the constant antibody dose of 7 mg/kg and siRNA dose of 1.4 mg/kg. After 14 or 28 days, gastrocnemius (gastroc) tissue was harvested and snap-frozen in liquid nitrogen. mRNA knockdown in target tissue was determined using a comparative qPCR assay. Total RNA was extracted from the tissue, reverse transcribed and mRNA levels were quantified using TaqMan qPCR, using the appropriately designed primers and probes. PPIB (housekeeping gene) was used as an internal RNA loading control, and the results were calculated by the comparative Ct method, where the difference between the target gene Ct value and the PPIB Ct value (ΔCt) was calculated and then further normalized relative to the PBS control group by taking a second difference (ΔΔCt).

### Results

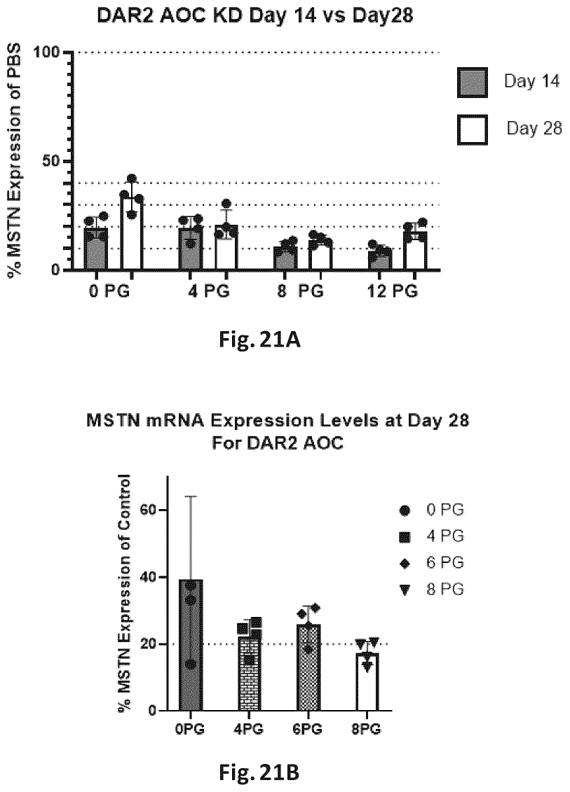

In vivo activities of DAR2 AOC with siRNA modified with 0, 4, 6, 8, and 12 PG linkages on the passenger strand and unmodified guide strands were evaluated for knock down activities of the MSTN mRNA levels at day 14 or day 28 in muscle cells from the gastrocnemius tissue (FIG. 21A) or at Day 28 (FIG. 21B).

The siRNAs with 8 or more PG linkages (8 PG and 12 PG) on the passenger strand of the DAR2 AOC increased the knock down activities of the siRNA at day 14 or day 28 of the siRNA when compared to the unmodified siRNA. The passenger strand of the siRNAs modified with 8 linkages had the most knock down activities among the group of modified siRNAs at day 14 or day 28 (FIG. 21A). The increasing number of PG on the passenger strand of the modified siRNA correlate with decreasing of MSTN mRNA levels or increasing knock down activities by the modified siRNA (FIG. 21B). At day 28, the modified siRNA with the passenger strand having the 8 PG pattern had better knock down activities than the ones for siRNA modified with the 4 PG, 6 PG, or 12 PG pattern.

Taken together the DAR2 AOC with the siRNAs modified with 8 PG on the passenger strand had the highest knockdown activities.

### Example 10 In Vivo Activity of DAR2 Anti-Transferrin Receptor 1 mAb Conjugated to MSTN siRNA Modified with PG Internucleotide Linkages on Both Strands

The siRNAs are made and characterized as described in Example 1. The siRNAs are modified on the passenger strand with 4 phosphoryl guanidine (PG) linkages and on the guide strand with 2 PG linkages. The DAR2 AOC or conjugates are synthesized by conjugating two siRNAs to an anti-TfR1 antibody as described in Example 1.

The conjugates are assessed for their ability to mediate MSTN or SSB mRNA downregulation in muscle tissues in wild type CD-1 mice. Mice are dosed via intravenous (iv) injection with PBS vehicle control and the indicated DAR2 AOC at the constant antibody dose. After 28 days, gastrocnemius (gastroc) tissue is harvested and snap-frozen in liquid nitrogen. mRNA knockdown in target tissue is determined using a comparative qPCR assay. Total RNA is extracted from the tissue, and reverse transcribed mRNA levels are quantified using TaqMan qPCR, using the appropriately designed primers and probes. PPIB (housekeeping gene) is used as an internal RNA loading control, and the results are calculated by the comparative Ct method, where the difference between the target gene Ct value and the PPIB Ct value (ΔCt) is calculated and then further normalized relative to the PBS control group by taking a second difference (ΔΔCt).

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A conjugate of Formula (I): 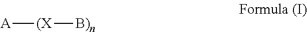
wherein,
A is a binding moiety;
B is a double-stranded oligonucleotide comprising a guide strand and a passenger strand;
X is a bond or a linker;
n is a number≥2; and
wherein the guide strand or the passenger strand comprises at least one modified internucleotide linkage comprising the structure of Formula (II): 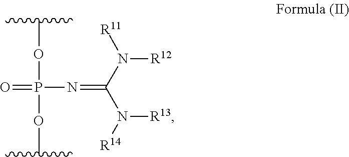
wherein each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl;
optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine.

2. (canceled)

3. The conjugate of claim 1, wherein the internucleotide linkage has the structure of Formula (III):

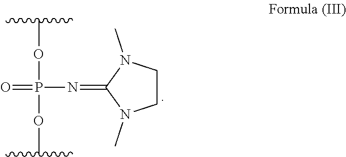 [SMILES: *OP(=O)(N=C1N(C)CCN1C)O*]

4. (canceled)

5. (canceled)

6. The conjugate of claim 1, wherein the internucleotide linkage has the structure of Formula (IV):

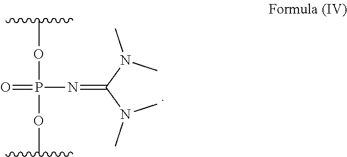 [SMILES: *OP(=O)(N=C(N(C)C)N(C)C)O*]

7. The conjugate of claim 1, wherein the at least one modified internucleotide linkage of Formula (II) is located at an internal position or a terminus of the guide strand.

8. (canceled)

9. (canceled)

10. The conjugate of claim 1, wherein the guide strand further comprises at least one phosphorothioate internucleotide linkage.

11. The conjugate of claim 10, wherein the at least one phosphorothioate internucleotide linkage and the at least one modified internucleotide linkage comprising the structure of Formula (II) are adjacent to one another.

12. The conjugate of claim 1, wherein the passenger strand comprises the at least one modified internucleotide linkage of Formula (II).

13. (canceled)

14. (canceled)

15. The conjugate of claim 1, wherein the at least one modified internucleotide linkage of Formula (II) is located at an internal position or a terminus of the passenger strand.

16. (canceled)

17. (canceled)

18. The conjugate of claim 1, wherein the passenger strand comprises two modified internucleotide linkages of Formula (II) at the 3′ end and two modified internucleotide linkages of Formula (II) at the 5′ end.

19. The conjugate of claim 1, wherein the passenger strand comprises four modified internucleotide linkages of Formula (II) at the 3′ end and four modified internucleotide linkages of Formula (II) at the 5′ end.

20. The conjugate of claim 1, wherein the passenger strand comprises 18 modified internucleotide linkages of Formula (II).

21. The conjugate of claim 1, wherein the passenger strand comprises two modified internucleotide linkages of Formula (II) at the 3′ end and two modified internucleotide linkages of Formula (II) at the 5′ end, and wherein the guide strand comprises two modified internucleotide linkages of Formula (II) at the 3′ end and two modified internucleotide linkages of Formula (II) at the 5′ end.

22.-26. (canceled)

27. The conjugate of claim 1, wherein the double-stranded oligonucleotide further comprises a modified internucleotide linkage selected from an alkylphosphonate, a triester, or a mesyl phosphoramidiate.

28.-34. (canceled)

35. The conjugate of claim 1, wherein the binding moiety is selected from the group consisting of a polypeptide, a protein, or an antibody or antigen binding fragment thereof.

36. (canceled)

37. (canceled)

38. The conjugate of claim 35, wherein the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof.

39. The conjugate of claim 1, wherein the conjugate has a drug to antibody ratio (DAR) of about 2:1, 3:1 or 4:1.

40. The conjugate of claim 1, wherein the modified internucleotide linkage of Formula (II) on the guide strand or the passenger strand increases activity of the conjugate of Formula (I).

41. The conjugate of claim 1, wherein the modified internucleotide linkage of Formula (II) on the guide strand or the passenger strand of the siRNA increases the stability of the conjugate of Formula (I).

42. A method of modulating mRNA expression levels of a gene in a subject, the method comprising:
a. providing the conjugate of claim 1; and
b. administering to the subject the conjugate, wherein the conjugate decreases mRNA expression levels of the gene in the subject.

43. (canceled)

44. (canceled)

45. A method of treating muscle atrophy or myotonic dystrophy in a subject in need thereof, the method comprising:
a. providing the conjugate of claim 1; and
b. administering to the subject the conjugate, wherein the conjugate mediates RNA interference against a target mRNA in the subject, thereby treating muscle atrophy or mytonic dystrophy in the subject.


SEQUENCE LISTING (49 sequences)

SEQ ID NO: 1 - RNA, 21 nt, synthetic construct
  source 1..21: mol_type = other RNA
  modified_base 1: mod_base = OTHER; note = (E)-vinyl phosphonate 2'-O-methoxyethoxy uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..21: mod_base = OTHER; note = phosphorothioate internucleotide linkage
ttattatttgttctttgcctt

SEQ ID NO: 2 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
ggcaaagaacaaataataa

SEQ ID NO: 3 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..21: mod_base = OTHER; note = phosphorothioate internucleotide linkage
ttattatttgttctttgcctt

SEQ ID NO: 4 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..2: mod_base = OTHER; note = phosphorothioate internucleotide linkage
  modified_base 19..20: mod_base = OTHER; note = phosphoryl guanidine internucleotide linkage
  modified_base 20..21: mod_base = OTHER; note = phosphorothioate internucleotide linkage
ttattatttgttctttgcctt

SEQ ID NO: 5 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..21: mod_base = OTHER; note = phosphoryl guanidine internucleotide linkage
ttattatttgttctttgcctt

SEQ ID NO: 6 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..2: mod_base = OTHER; note = phosphorothioate internucleotide linkage
  modified_base 2..3: mod_base = OTHER; note = phosphoryl guanidine internucleotide linkage
  modified_base 19..21: mod_base = OTHER; note = phosphoryl guanidine internucleotide linkage
ttattatttgttctttgcctt

SEQ ID NO: 7 - RNA, 21 nt, synthetic construct
  source 1..21: mol_type = other RNA
  modified_base 1: mod_base = OTHER; note = 2'-O-methyl ribonucleotide
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphoryl guanidine internucleotide linkage
  modified_base 19..21: mod_base = OTHER; note = phosphoryl guanidine internucleotide linkage
ttattatttgttctttgcctt

SEQ ID NO: 8 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
ttattatttgttctttgcctt

SEQ ID NO: 9 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..4: mod_base = OTHER; note = phosphoryl guanidine internucleotide linkage
  modified_base 16..19: mod_base = OTHER; note = phosphoryl guanidine internucleotide linkage
ggcaaagaacaaataataa

SEQ ID NO: 10 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..5: mod_base = OTHER; note = phosphoryl guanidine internucleotide linkage
  modified_base 15..19: mod_base = OTHER; note = phosphoryl guanidine internucleotide linkage
ggcaaagaacaaataataa

SEQ ID NO: 11 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..6: mod_base = OTHER; note = phosphoryl guanidine internucleotide linkage
  modified_base 14..19: mod_base = OTHER; note = phosphoryl guanidine internucleotide linkage
ggcaaagaacaaataataa

SEQ ID NO: 12 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 13..19: mod_base = OTHER; note = phosphoryl guanidine internucleotide linkage
ggcaaagaacaaataataa

SEQ ID NO: 13 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..8: mod_base = OTHER; note = phosphoryl guanidine internucleotide linkage
  modified_base 12..19: mod_base = OTHER; note = phosphoryl guanidine internucleotide linkage
ggcaaagaacaaataataa

SEQ ID NO: 14 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..9: mod_base = OTHER; note = phosphoryl guanidine internucleotide linkage
  modified_base 11..19: mod_base = OTHER; note = phosphoryl guanidine internucleotide linkage
ggcaaagaacaaataataa

SEQ ID NO: 15 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..19: mod_base = OTHER; note = phosphoryl guanidine internucleotide linkage
ggcaaagaacaaataataa

SEQ ID NO: 16 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 17..19: mod_base = OTHER; note = phosphorothioate internucleotide linkage
ggcaaagaacaaataataa

SEQ ID NO: 17 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphorothioate internucleotide linkage
  modified_base 17..19: mod_base = OTHER; note = phosphoryl guanidine internucleotide linkage
ggcaaagaacaaataataa

SEQ ID NO: 18 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 17..19: mod_base = OTHER; note = phosphoryl guanidine internucleotide linkage
ggcaaagaacaaataataa

SEQ ID NO: 19 - RNA, 21 nt, synthetic construct
  source 1..21: mol_type = other RNA
ttattatttgttctttgcctt

SEQ ID NO: 20 - RNA, 21 nt, synthetic construct
  source 1..21: mol_type = other RNA
ttacattaaagtctgttgttt

SEQ ID NO: 21 - RNA, 21 nt, synthetic construct
  source 1..21: mol_type = other RNA
  modified_base 1: mod_base = OTHER; note = (E)-vinyl phosphonate 2'-O-methoxyethoxy uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..21: mod_base = OTHER; note = phosphorothioate internucleotide linkage
ttattatttgttctttgcctt

SEQ ID NO: 22 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..21: mod_base = OTHER; note = phosphorothioate internucleotide linkage
ttattatttgttctttgcctt

SEQ ID NO: 23 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphorothioate internucleotide linkage
  modified_base 17..19: mod_base = OTHER; note = phosphorothioate internucleotide linkage
ggcaaagaacaaataataa

SEQ ID NO: 24 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 17..19: mod_base = OTHER; note = phosphoryl guanidine internucleotide linkage
ggcaaagaacaaataataa

SEQ ID NO: 25 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..5: mod_base = OTHER; note = phosphoryl guanidine internucleotide linkage
  modified_base 15..19: mod_base = OTHER; note = phosphoryl guanidine internucleotide linkage
ggcaaagaacaaataataa

SEQ ID NO: 26 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 13..19: mod_base = OTHER; note = phosphoryl guanidine internucleotide linkage
ggcaaagaacaaataataa

SEQ ID NO: 27 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..8: mod_base = OTHER; note = phosphoryl guanidine internucleotide linkage
  modified_base 12..19: mod_base = OTHER; note = phosphoryl guanidine internucleotide linkage
ggcaaagaacaaataataa

SEQ ID NO: 28 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..20: mod_base = OTHER; note = phosphorothioate internucleotide linkage
  modified_base 20..21: mod_base = OTHER; note = phosphoryl guanidine internucleotide linkage
ttattatttgttctttgcctt

SEQ ID NO: 29 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..20: mod_base = OTHER; note = phosphoryl guanidine internucleotide linkage
  modified_base 20..21: mod_base = OTHER; note = phosphorothioate internucleotide linkage
ttattatttgttctttgcctt

SEQ ID NO: 30 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 17..19: mod_base = OTHER; note = phosphorothioate internucleotide linkage
ggcaaagaacaaataataa

SEQ ID NO: 31 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphorothioate internucleotide linkage
  modified_base 17..19: mod_base = OTHER; note = phosphoryl guanidine internucleotide linkage
ggcaaagaacaaataataa

SEQ ID NO: 32 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..5: mod_base = OTHER; note = phosphoryl guanidine internucleotide linkage
  modified_base 15..19: mod_base = OTHER; note = phosphoryl guanidine internucleotide linkage
ggcaaagaacaaataataa

SEQ ID NO: 33 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 13..19: mod_base = OTHER; note = phosphoryl guanidine internucleotide linkage
ggcaaagaacaaataataa

SEQ ID NO: 34 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..8: mod_base = OTHER; note = phosphoryl guanidine internucleotide linkage
  modified_base 12..19: mod_base = OTHER; note = phosphoryl guanidine internucleotide linkage
ggcaaagaacaaataataa

SEQ ID NO: 35 - RNA, 18 nt, synthetic construct
  source 1..18: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..18: mod_base = OTHER; note = 2'-O-methyl ribonucleotide
  modified_base 1..3: mod_base = OTHER; note = phosphoryl guanidine internucleotide linkage
  modified_base 4..5: mod_base = OTHER; note = phosphoryl guanidine internucleotide linkage
  modified_base 6..7: mod_base = OTHER; note = phosphoryl guanidine internucleotide linkage
  modified_base 13..14: mod_base = OTHER; note = phosphoryl guanidine internucleotide linkage
  modified_base 15..18: mod_base = OTHER; note = phosphoryl guanidine internucleotide linkage
ggcaaagaacaaatataa

SEQ ID NO: 36 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 5..7: mod_base = OTHER; note = phosphoryl guanidine internucleotide linkage
  modified_base 13..15: mod_base = OTHER; note = phosphoryl guanidine internucleotide linkage
  modified_base 17..19: mod_base = OTHER; note = phosphoryl guanidine internucleotide linkage
ggcaaagaacaaataataa

SEQ ID NO: 37 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..3: mod_base = OTHER; note = phosphoryl guanidine internucleotide linkage
  modified_base 13..19: mod_base = OTHER; note = phosphoryl guanidine internucleotide linkage
ggcaaagaacaaataataa

SEQ ID NO: 38 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 17..19: mod_base = OTHER; note = phosphoryl guanidine internucleotide linkage
ggcaaagaacaaataataa

SEQ ID NO: 39 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1: mod_base = OTHER; note = 5' phosphoryl guanidine linkage
  modified_base 1..5: mod_base = OTHER; note = phosphoryl guanidine internucleotide linkage
  modified_base 15..19: mod_base = OTHER; note = phosphoryl guanidine internucleotide linkage
ggcaaagaacaaataataa

SEQ ID NO: 40 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1: mod_base = OTHER; note = 5' phosphoryl guanidine linkage
  modified_base 1..3: mod_base = OTHER; note = phosphorothioate internucleotide linkage
  modified_base 17..19: mod_base = OTHER; note = phosphorothioate internucleotide linkage
ggcaaagaacaaataataa

SEQ ID NO: 41 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 13..19: mod_base = OTHER; note = phosphoryl guanidine internucleotide linkage
ggcaaagaacaaataataa

SEQ ID NO: 42 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..6: mod_base = OTHER; note = phosphoryl guanidine internucleotide linkage
  modified_base 11..19: mod_base = OTHER; note = phosphoryl guanidine internucleotide linkage
ggcaaagaacaaataataa

SEQ ID NO: 43 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1: mod_base = OTHER; note = 5' phosphoryl guanidine linkage
  modified_base 1..6: mod_base = OTHER; note = phosphoryl guanidine internucleotide linkage
  modified_base 11..19: mod_base = OTHER; note = phosphoryl guanidine internucleotide linkage
ggcaaagaacaaataataa

SEQ ID NO: 44 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..6: mod_base = OTHER; note = phosphoryl guanidine internucleotide linkage
  modified_base 10..19: mod_base = OTHER; note = phosphoryl guanidine internucleotide linkage
ggcaaagaacaaataataa

SEQ ID NO: 45 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 11..19: mod_base = OTHER; note = phosphoryl guanidine internucleotide linkage
ggcaaagaacaaataataa

SEQ ID NO: 46 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1..7: mod_base = OTHER; note = phosphoryl guanidine internucleotide linkage
  modified_base 10..19: mod_base = OTHER; note = phosphoryl guanidine internucleotide linkage
ggcaaagaacaaataataa

SEQ ID NO: 47 - RNA, 19 nt, synthetic construct
  source 1..19: mol_type = other RNA
  modified_base 1..6: mod_base = OTHER; note = 2'-O-methyl ribonucleotide
  modified_base 7..9: mod_base = OTHER; note = 2'-fluoro ribonucleotide
  modified_base 10..19: mod_base = OTHER; note = 2'-O-methyl ribonucleotide
  modified_base 1: mod_base = OTHER; note = 5' phosphoryl guanidine linkage
  modified_base 1..7: mod_base = OTHER; note = phosphoryl guanidine internucleotide linkage
  modified_base 10..19: mod_base = OTHER; note = phosphoryl guanidine internucleotide linkage
ggcaaagaacaaataataa

SEQ ID NO: 48 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..20: mod_base = OTHER; note = phosphorothioate internucleotide linkage
  modified_base 20..21: mod_base = OTHER; note = phosphoryl guanidine internucleotide linkage
ttattatttgttctttgcctt

SEQ ID NO: 49 - RNA, 21 nt, synthetic construct
  source 1..21: mol_type = other RNA
  modified_base 1: mod_base = OTHER; note = (E)-vinyl phosphonate 2'-O-methoxyethoxy-2',3'-unlocked uridine
  modified_base 2: mod_base = OTHER; note = 2'-fluoro ribonucleotide
  modified_base 3..5: mod_base = OTHER; note = 2'-O-methyl ribonucleotide
  modified_base 6: mod_base = OTHER; note = 2'-fluoro ribonucleotide
  modified_base 7..13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17..21: mod_base = OTHER; note = 2'-O-methyl ribonucleotide
  modified_base 1..3: mod_base = OTHER; note = phosphorothioate internucleotide linkage
  modified_base 19..20: mod_base = OTHER; note = phosphoryl guanidine internucleotide linkage
  modified_base 20..21: mod_base = OTHER; note = phosphorothioate internucleotide linkage
ttattatttgttctttgcctt